(19) United States
(12) Patent Application Publication
(10) Pub. No.: US 2026/0035418 A1
(43) Pub. Date: Feb. 5, 2026

(54) TREATMENTS FOR CELLULAR REJUVENATION

(71) Applicant: President and Fellows of Harvard College, Cambridge, MA (US)

(72) Inventors: Denitsa M. Milanova, Cambridge, MA (US); George M. Church, Cambridge, MA (US); Madison Ski Krieger, Cambridge, MA (US); David B Thompson, Cambridge, MA (US)

(73) Assignee: President and Fellows of Harvard College, Cambridge, MA (US)

(21) Appl. No.: 18/998,219

(22) PCT Filed: Aug. 4, 2023
(86) PCT No.: PCT/US2023/071689

§ 371 Date: Jan. 24, 2025

Related U.S. Application Data
(60) Provisional application No. 63/395,398, filed on Aug. 5, 2022.

(51) Int. Cl. C07K 14/47; C12N 15/86; G01N 33/50; G16B 40/20

(52) U.S. Cl. CPC C07K 14/47; C12N 15/86; G01N 33/5091; G16B 40/20; C12N 2750/14143; G01N 2800/7042

(57) ABSTRACT

The present disclosure relates to a method of inducing cellular rejuvenation and/or regeneration of a cell or tissue comprising contacting the cell with an effective amount of a protein or nucleic acid encoding a protein that induces cellular rejuvenation and/or regeneration of a cell or tissue. The present disclosure describes cell or tissue prepared according to methods described herein. The present disclosure also provides for methods of treating patients using cell or tissue generated by the methods described herein. The present disclosure also provides methods and systems for identifying a perturbant capable of changing a cell's state, function, and predicted age from an old reference state to a younger altered state.

### CROSS-REFERENCE TO RELATED APPLICATIONS

This application claims the benefit under 35 U.S.C. § 119(e) of U.S. Provisional Application No. 63/395,398, filed Aug. 5, 2022, the contents of which is hereby incorporated by reference in its entirety.

### REFERENCE TO AN ELECTRONIC SEQUENCE LISTING

The contents of the electronic sequence listing (H049870767WO00-SEQ-KVC.xml; Size: 29,231 bytes; and Date of Creation: Aug. 2, 2023) is herein incorporated by reference in its entirety.

### BACKGROUND

Aging affects the biology of a cell systemically, and true reversal of aging would counter these same systemic changes. Such changes manifest in specific functional defects, but while the ultimate value of rejuvenating technologies would derive from rescue of functionality, no single functional metric reports on the totality of the aged cell state. Meanwhile, observation of time-resolved changes during aging, a seemingly direct way of detecting mechanistic drivers of ageing is itself not straightforward. Our impression of a monotonic decline in cell, tissue, and bodily function with age, even when accurate, may be the result of dynamic and non-linear molecular processes, with no seeming correlation between cause and effect.

Now-classic work using cell fusion and heterokaryon models established that factors present in young cells can overcome aged cell states and reverse certain aged cell behaviors. These studies used the tools of their time, and ultimately the specific molecular drivers of this seeming age-reversal were difficult to identify. However, the existence of such rejuvenating factors in young cells, and the apparent plasticity of the aged cell phenotype, suggests the possibility of genetically-encodable rejuvenation factors. Thus, the concept of cellular rejuvenation can be achieved by (epi)genetic gain-of-function therapies.

Dermal fibroblasts are cells that synthesize extracellular matrix (ECM) proteins, thus producing the structural framework of the skin. Fibroblasts are also involved in the process of wound healing and tissue regeneration. Primary human dermal fibroblasts (HDFs) isolated from different-aged donors are readily distinguished by their doubling time and replicative capacity, cell size, and ECM protein production. And ultimately, the decline of HDF with age impacts skin health with macroscopically visible results, pointing directly to applications of rejuvenative interventions in medical aesthetic indications.

A number of skin therapeutic products have been developed for improving the appearance of human skin. Surgical interventions can be effective with respect to reducing appearance of aging but are invasive, inconvenient, and expensive. Minimally invasive methods are available, but such methods are generally less effective. For instance, energy-based methods including lasers, radiofrequency, and ultrasound can be effective at improving the skin texture, but such tissue ablative methods require longer patient healing times. Other methods include injections of neurotoxins and facial fillers. Neither of these methods address the underlying aging phenotype on a molecular, cellular or functional level.

Historically, the discovery of novel rejuvenative therapies has been hypothesis-driven, limited in throughput, and focused on a singular mechanism, aging hallmark or biological pathway. Accordingly, there is a need for improved screening and target verification that offer increased throughput and phenotypic readout, while providing rapid age prediction and classification of functional cell state profiles.

### SUMMARY

The present disclosure provides methods for identifying genes and proteins capable of changing a cell's state, function, and predicted age. Further, the present disclosure provides methods for identifying an in vitro cell's state, function, and predicted age.

Aspects of the present disclosure relate to a method, comprising: contacting a cell with an effective amount of a protein or a nucleic acid encoding the protein, wherein the protein is: ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof; wherein the effective amount is sufficient to induce cellular rejuvenation and/or regeneration of the cell.

Aspects of the present disclosure relate to a method, comprising: administering to a subject an effective amount of a protein or a nucleic acid encoding the protein, wherein the protein is: ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IP05, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof; wherein the effective amount is sufficient to induce cellular rejuvenation of a cell in the subject.

In some embodiments, the protein is selected from the group consisting of: ANXA2, PRRC2B, MAP4, BGN, CLTA, RPS23, AKT1, VCP, FAM129B, LMAN1, GUK1, SEC61A1, ELL2, RPN1, KIF5B, MSN, and TGOLN2. In some embodiments, the protein is selected from the group consisting of: ANXA2, PRRC2B, MAP4, BGN, CLTA, and RPS23. In some embodiments, the protein is ANXA2. In some embodiments, the protein is PRRC2B. In some embodiments, the protein is MAP4. In some embodiments, the protein is BGN. In some embodiments, the protein is CLTA. In some embodiments, the protein is RPS23.

In some embodiments, the effective amount is sufficient to reduce reactive oxygen species (ROS) abundance in the cell, compared to an untreated cell. In some embodiments, the ROS abundance in the cell is reduced by at least 1%, at least 2%, at least 3%, at least 4%, at least 5%, at least 10%, at least 15%, at least 20%, or at least 25%, compared to an untreated cell. In some embodiments, the effective amount is sufficient to increase the growth rate of the cell, compared to an untreated cell. In some embodiments, the growth rate of the cell is increased by at least 1%, at least 2%, at least 3%, at least 4%, at least 5%, at least 10%, at least 15%, at least 20%, at least 25%, at least 30%, at least 35%, or at least 40%, compared to an untreated cell.

In some embodiments, two or more proteins are selected, wherein the two or more proteins are encoded by two or more nucleic acids and the two or more nucleic acids are separated by a polycistronic element. In some embodiments, the polycistronic element is an IRES or a 2A sequence.

In some embodiments, the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years. In some embodiments, average cellular rejuvenation is defined as the product of (A) the fraction of treated, misclassified cells in a supervised two-class machine learning ensemble, wherein an untreated training population of each class exceeds 1,000 cells; and (B) the age difference between the two classes.

In some embodiments, the effective amount is sufficient to increase the signal intensity of cytoplasmic actin stained with phalloidin and measured by microscopy, the signal intensity of the Golgi apparatus/apparati stained with wheat germ agglutinin and measured by microscopy, the signal intensity of the plasma membrane stained with wheat germ agglutinin and measured by microscopy, the signal intensity of extranuclear DNA stained with Hoechst 33342 and measured by microscopy, the signal intensity of mitochondria stained with MitoTracker Deep Red and measured by microscopy, the signal intensity of the endoplasmic reticulum stained with concanavalin A and measured by microscopy, or the peripheral cellular signal intensity of RNA stained with SYTO 14 and measured by microscopy.

In some embodiments, the effective amount is sufficient to decrease cytoplasmic volume as measured by microscopy, decrease cell volume as measured by microscopy, decrease cell surface area as measured by microscopy, or decrease the signal intensity of nuclear DNA stained with Hoechst 33342 and measured by microscopy. In some embodiments, the effective amount is sufficient to increase replicative life span of the cell by at least 25%, at least 30%, at least 35%, at least 40%, at least 45%, or at least 50%. In some embodiments, the effective amount is sufficient to increase total gene expression or extracellular matrix gene expression or collagen expression levels of the cell by at least 25%, at least 30%, at least 35%, at least 40%, at least 45%, or at least 50%. In some embodiments, the effective amount is sufficient to induce an average cellular rejuvenation of at least 20 years.

In some embodiments, the cell is an adult stem cell. In some embodiments, the adult stem cell is selected from a hematopoietic stem cell, epithelial stem cell, neuronal stem cell and mesenchymal stem cell. In some embodiments, the cell is selected from an ectoderm, endoderm, mesoderm and germ cell. In some embodiments, the cell is an adult stem cell. In some embodiments, the adult stem cell is hematopoietic stem cell, epithelial stem cell, neuronal stem cell or mesenchymal stem cell. In some embodiments, the mesenchymal stem cell is fibroblast, myocyte, adipocyte, chondrocyte, or osteocyte. In some embodiments, the hematopoietic stem cell is a T cell or NK cell. In some embodiments, the T cell is a CD4⁺CD8⁺ cell, CD4⁺ cell (Th1, Th2, Th17, or Treg), a naive T cell, a central memory T cell, or an effector memory T cell. In some embodiments, the cell is an ectoderm, endoderm, mesoderm or germ cell. In some embodiments, the ectoderm cell is keratinocyte, pigment cell or neuronal cell. In some embodiments, the endoderm cell is a liver cell, lung cell, pancreatic cell or thyroid cell. In some embodiments, the mesoderm cell is a cardiac muscle cell, skeletal muscle cell, smooth muscle cell, kidney tubule cell or a red blood cell. In some embodiments, the germ cell is an egg or sperm cell.

In some embodiments, the cell is selected from the group consisting of: fibroblasts, hematopoietic stem cells, endothelial cells, chondrocytes, skeletal muscle stem cells, keratinocytes, mesenchymal stem cells and corneal epithelial cells. In some embodiments, the cells are fibroblasts. In some embodiments, the fibroblasts are human dermal fibroblasts. In some embodiments, the protein is a human, canine, feline, bovine, ovine, caprine, equine, murine, porcine, or pachyderm protein. In some embodiments, the protein is delivered to skin tissue layers and structures including stratum corneum, epidermis, basement membrane, dermis, hair follicles, blood vessels, and sebaceous glands or and eccrine glands.

In some embodiments, the nucleic acid comprises a heterologous promoter operably linked to an open reading frame. In some embodiments, the heterologous promoter is a constitutive promoter or an inducible promoter. In some embodiments, the regulatory sequence comprises a cell-specific promoter or a tissue-specific promoter. In some embodiments, the regulatory sequence comprises a promoter selected from the group consisting of: an hEfla promoter, an shEfla promoter (or truncated hEfla promoter), a CAG promoter (such as cytomegalovirus, chicken beta-actin intron, splice acceptor of the rabbit beta-globin gene), a CMV promoter, an hAAT promoter, a thyroid hormone-binding globulin promoter, an albumin promoter, a thyroxin-binding globulin (TBG) promoter, a hepatic control region (HCR)-ApoCII hybrid promoter, a CASI promoter, an HCR-hAAT hybrid promoter, an hAAT promoter combined with mouse albumin gene enhancer (Ealb) element, and an apolipoprotein E promoter.

In some embodiments, the nucleic acid is operably linked to a 3′ untranslated region for RNA stability and expression in mammalian cells. In some embodiments, the 3′ untranslated region comprises a WPRE sequence, a WPRE3 sequence, an SV40 late polyadenylation signal (e.g., truncated), an HBG polyadenylation signal, a rabbit beta-globin polyadenylation signal, a bovine bgpA, an ETC polyadenylation signal, or any combination thereof.

In some embodiments, the protein is delivered in a viral vector. In some embodiments, the nucleic acid is delivered in a viral vector. In some embodiments, the viral vector is an AAV vector. In some embodiments, the AAV vector is derived from an AAV serotype selected from the group consisting of: AAV1, AAV2, AAV3, AAV4, AAV5, AAV6, AAV6.2, AAV7, AAV8, AAV9, AAV10, AAV11, AAV12, AAV13, AAVrh8, AAVrh10, and AAVrh32. In some embodiments, the AAV vector is derived from an AAV6 serotype. In some embodiments, the AAV vector is derived from an AAV2 serotype.

In some embodiments, the method further comprises contacting the cell with the protein or administering the protein directly to the subject. In some embodiments, the method further comprises contacting the cell with the protein or administering to the subject the nucleic acid comprising an open reading frame encoding the protein. In some embodiments, the nucleic acid is delivered in a non-viral vector or a viral vector. In some embodiments, the contacting comprises transfecting the cell.

In some embodiments, the nucleic acid comprises DNA, RNA, or a combination thereof. In some embodiments, the protein comprises a sequence that is at least 90% identical to any one of SEQ ID NOs: 1-6. In some embodiments, the nucleic acid comprises an amino acid sequence that is at least 90% identical to any one of SEQ ID NOs: 7-12.

Aspects of the present disclosure relate to a method, comprising: overexpressing in a cell a nucleic acid encoding a protein, wherein the protein is: ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof; wherein the overexpressing produces an effective amount of the protein in the cell, and wherein the effective amount is sufficient to induce cellular rejuvenation in the cell. In some embodiments, the protein is selected from ANXA2, PRRC2B, MAP4, BGN, CLTA, RPS23, AKT1, VCP, FAM129B, LMAN1, GUK1, SEC61A1, ELL2, RPN1, KIF5B, MSN, and TGOLN2.

In some embodiments, the protein is an endogenous protein and the method comprises activating expression or activity of the endogenous protein at a level that is higher than a baseline level. In some embodiments, the subject has been diagnosed or is at risk of being diagnosed with a skin condition. In some embodiments, the skin condition is photoaging, intrinsic aging, aging induced by repeated facial expressions, loss of tone and gravity manifested in rough texture, sagging, wrinkles, furrows, folds, or any combinations thereof.

Aspects of the present disclosure relate to a cell, comprising: an engineered nucleic acid encoding a protein, wherein the protein is: ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof. In some embodiments, the protein is selected from the group consisting of: ANXA2, PRRC2B, MAP4, BGN, CLTA, RPS23, AKT1, VCP, FAM129B, LMAN1, GUK1, SEC61A1, ELL2, RPN1, KIF5B, MSN, and TGOLN2.

In some embodiments, the protein is a recombinant purified peptide sequence. In some embodiments, the purified peptide is introduced to cells through non-covalent complexation with protein, lipid, synthetic polymeric, cell-binding ligand agents, or pore-forming agents. In some embodiments, the purified peptide is introduced to cells through chemical formulations which induce entry into cells through osmotic shock. In some embodiments, the purified peptide is introduced to cells through chemical formulations which destabilize cellular membranes.

In some embodiments, the cell is a fibroblast. In some embodiments, the fibroblast is a human dermal fibroblast. In some embodiments, the cell is a stem cell. In some embodiments, the stem cell is selected from the group consisting of: hematopoietic stem cells, skeletal muscle stem cells, and mesenchymal stem cells. In some embodiments, the stem cell is a human induced pluripotent stem cell. In some embodiments, the cell is selected from the group consisting of: endothelial cells, chondrocytes, keratinocytes, and corneal epithelial cells. In some embodiments, the cell expresses the protein at a level that is higher than a baseline level.

Aspects of the present disclosure relate to a pharmaceutical composition comprising any one of the cells described herein and a pharmaceutically-acceptable excipient and/or polymeric carrier.

Aspects of the present disclosure relate to a pharmaceutical composition comprising: a recombinant vector genome comprising one or more transgenes encoding one or more polypeptide sequences selected from ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, and HSPA9, wherein the vector genome is configured to express the one or more transgene at a level sufficient to induce tissue rejuvenation and/or regeneration; and a pharmaceutically-acceptable excipient and/or polymeric carrier.

Aspects of the present disclosure relate to a method, comprising administering the pharmaceutical composition described herein to skin of a subject via intraepidermal, transepidermal, intradermal, transdermal, subcutaneous, intramuscular, or topical administration. In some embodiments, the skin exhibits signs of aging. In some embodiments, the signs of aging are indicative of a skin condition selected from photoaging, intrinsic aging, aging induced by repeated facial expressions, loss of tone and gravity manifested in rough texture, sagging, wrinkles, furrows, folds, and any combinations thereof.

Aspects of the present disclosure relate to methods for measuring the age of a cell, comprising: contacting a cell with a first reagent capable of recognizing cytoplasmic actin, with a second reagent capable of recognizing Golgi apparatus/apparati, with a third reagent capable of recognizing plasma membrane, with a fourth reagent capable of recognizing extranuclear DNA, with a fifth reagent capable of recognizing mitochondria, with a fifth reagent capable of recognizing endoplasmic reticulum, with a sixth reagent capable of recognizing RNA; determining the cell's morphological and/or functional fingerprint based on the intensity of a signal associated with the first, second, third, fourth, fifth, and sixth binding reagent and the intensity of a signal associated with the at least second binding reagent; and identifying the cell's age based upon a machine learning algorithm of extracted features. In some embodiments, the age of the cell is further measured using a machine learning algorithm of a cell's expression features in combination with morphological and/or functional features.

Aspects of the present disclosure relate to methods for defining a rejuvenation index, called a ‘Youth score’, wherein the cell is contacted with a perturbant which induces a cell's transition from aged (reference) cell state to younger (altered) cell state, comprising: a change in a cell state, function and/or expression between the totality of unperturbed cells and the totality of perturbed cells; and comparing the cell's transition fingerprint thereby quantifying the cellular transition due to the perturbation.

In some embodiments, the protein is a fragment of the protein. In some embodiments, the protein is a modified version of the protein. In some embodiments, the protein is truncated. In some embodiments, the protein is an isoform of the protein.

### BRIEF DESCRIPTION OF THE DRAWINGS

FIG. 1 shows the experimental workflow of the method as described herein.

FIG. 2 shows results of an exemplary morphological profiling experiment using the CellPaint assay as described herein.

FIG. 3 shows exemplary “Youth Score” results of library members that emerge from machine learning analysis of CellPaint data as described herein.

FIG. 4 shows high-performing library members exhibiting strong fingerprint similarity to young cells as described herein.

FIG. 5 shows cell cycle of healthy cells.

FIG. 6 shows a t-distributed stochastic neighbor embedding (tSNE) projection of control library members, young cells, and old cells.

FIG. 7 shows a tSNE projection of high-performing library members, young cells, and old cells.

FIG. 8 shows the normalized frequency of reactive oxygen species in high-performing library members, as a measure of metabolism.

FIG. 9 shows the cellular growth rate in high-performing library members, as a measure of replicative life span.

FIG. 10 is a schematic illustrating the timeline of human skin rejuvenation using AAV-based gene therapy in an NSG skin xenograft mouse model.

FIG. 11 is a flowchart illustrating the whole transcriptome analysis of human skin graft for spatially-resolved mRNA sequencing across a whole fresh-frozen tissue section.

FIG. 12 is a percentage of cell types identified in control and treatment samples of Annexin A2 (ANXA2), Biglycan (BGN), and Clathrin light chain A (CLTA).

FIG. 13 shows representative images for of histological analysis by H&E staining and similarity between the tissue expression vector to score the likelihood of documented cell types to have populated the focal tissue site for a control and treatment groups of Annexin A2 (ANXA2), Biglycan (BGN), and Clathrin light chain A (CLTA). The distribution of fibroblast and keratinocyte cells was identified and further analyzed.

FIG. 14 shows volcano plots of spatial RNA-seq data of skin generated by comparing control and treated (treatment groups of ANXA2, BGN, and CLTA) human skin grafts in fibroblasts and keratinocyte cell populations. The differential expression gene (DEG) list cutoffs were defined as P_adj<0.05 and an averaged tissue expression log 2(Fold Change) of 0.25.

FIG. 15 shows Gene Ontology (GO) analysis of spatial RNA-seq data for biological processes of human skin grafts showing the top upregulated genes in fibroblast and keratinocyte cell populations. The GO biological processes list cutoffs were defined as Bonferroni<0.05 and a representative subset of the terms is derived using a clustering algorithm that relies on semantic similarity measures.

FIGS. 16A-16C shows gene expression profiles in treated and control groups. FIG. 16A shows upregulation of extracellular matrix organization genes, positive regulation of fibroblast proliferation genes, negative regulation of stem cell regulation genes, and wound healing genes in the fibroblast cells of treated skin grafts (treatment groups of ANXA2, BGN, and CLTA) compared to control skin grafts. Gene expression levels and spatial distribution of fibroblast markers Collagen 3 (COL3A1), Collagen 1 (COL1A1), Elastin (ELN), Vimentin (VIM), and basement membrane Collagen 17 (COL17A1) are shown for treated and control groups. FIG. 16B shows upregulation of keratinocyte differentiation genes, epidermis development genes, positive regulation of TOR signaling genes, and establishment of skin barrier genes in the keratinocyte cells of treated skin grafts (treatment groups of ANXA2, BGN, and CLTA) compared to control skin grafts. Gene expression levels and spatial distribution of epidermal progenitor and stem cell markers Alpha 6 (ITGA6), Beta 1 (ITGB1), Alpha 3 (ITGA3), and Keratin 14 (KRT14) are shown for treated and control groups. FIG. 16C shows upregulation of keratinocyte cell cycle genes, and chromatin organization genes in the keratinocyte cells of treated skin grafts (treatment groups of ANXA2, BGN, and CLTA) compared to control skin grafts.

FIG. 17 shows fraction of fibroblast and keratinocyte cells in G2/M and S phases of the cell cycle for treatment (treatment groups of ANXA2, BGN, and CLTA) and control groups.

FIG. 18 shows volcano plots of spatial RNA-seq data of skin generated by comparing control and CLTA-treated human skin grafts in the melanocyte cell population. The differential expression gene (DEG) list cutoffs were defined as P_adj<0.05 and an averaged tissue expression log 2(Fold Change) of 0.25.

FIG. 19 shows a list of downregulated melanocyte genes in CLTA-treated human skin relative to the control population.

### DETAILED DESCRIPTION

Besides the toll of human suffering due to age-related disease, aging is expensive—the average American over 65 years old will incur $60,000 of medical costs in their last year of life alone, of which 65% will be subsidized by Medicare¹. Increasing average lifespan by even 1 year is estimated to be worth $38 trillion, and “a compression of morbidity that improves health is more valuable than further increases in life expectancy”².

The past few decades have seen major advances in the definition of “aging” as a disease state which can progress across all systems and scales in an organism, from sub-cellular functions up to entire-tissue dysfunctions, as well as major advances in the understanding of how and why the changes which cause aging are often observed at certain points along an organism's lifespan. While many critical discoveries have concerned mechanisms of aging at the level of the entire genome, such as the role of telomerase³ and histone methylation⁴, focus has recently turned to the importance of particular genes, gene networks, and pathways in controlling both healthspan, lifespan, and the progression of aging phenotypes⁵⁻⁷. Seeking out such networks presents a fundamental challenge in and of itself, even precluding the possibility of leveraging such networks to extend and improve human life. With a number of functional (coding and non-coding) genes numbering at least 20,000⁸, and a huge number of regulatory mechanisms⁹, such as epigenetics, gene regulation, transcription factors, and so forth, there are limitless building blocks with which to construct a meaningful network of interacting genes whose dysfunction could lead to aging and reversal to rejuvenation and/or regeneration. While tools and datasets exist which can tease out differentially expressed genes between old and young cohorts, there is still a large gap between such studies and the ability to draw an accurate network of genes that drive the expression level of other genes and using this network to describe and reverse aging.

While this is a large challenge, it is nonetheless a well-defined one. Sifting through the enormous combinatorial possibilities of genetic aging networks represents a Tukey paradigm¹⁰ in which one seeks to reduce the number of relevant possibilities to a number which can feasibly be tested in the lab (“explore”), find those which pass some threshold of credibility (“confirm”), and understand the biology of the resulting positives (“explain”). The exploratory phase is also well-defined due to the nature of aging. Aging is inherently a time-driven disorder; while critical changes may take place at different rates in different individuals, almost no relevant change takes place “instantaneously”. Most aging systems can therefore be viewed as a collection of time series, where each individual time series represents the expression level of a particular gene observed at a particular time (age). Fortuitously, the mathematical methods for inferring mechanistic interactions between such collections of time series have also expanded dramatically over the past twenty years. Among these inference tools, those suited to the task of identifying genetic aging networks must be model(/equation)-free and non-parametric (since a priori we have no knowledge of the biology underlying the network) as well as inductive (imposing no constraints on the type of network that one can infer, but instead constructing it on a connection-by-connection basis). As a convenient shorthand, we will refer to these tools as Empirical Dynamics Modeling (EDM). The fact that this umbrella term contains several different tools originating from different areas of mathematics is a strength—seeking consensus between techniques has been shown to give more accurate results in inferring biological networks¹¹ and also further reduces the dimensionality of the number of networks worthy of experimental validation¹².

Based on previous work using EDM techniques to understand protein interactions in blood disease¹³, an ensemble of techniques was selected as a prototype for inferring genetic aging networks. This prototype was tested on a dataset described in this disclosure: an aging time series of 5,138 genes expression levels in dermal fibroblasts, compiled from over 100 donors ranging in age from 1-96, which is publicly accessible¹⁴. This dataset was chosen as a benchmark because of its remarkable length of resolution (covering most of the range of current human lifespans) and because of its relative sparseness (between 0 and 5 donors per age), which further credits the program should it happen to produce positive results on such a small dataset. Dermal fibroblasts were furthermore chosen due to their accessibility as well as experimental considerations, but also for the potential to massively scale up future experimental investigations, due to the non-invasiveness of examining and extracting human skin.

In what follows, a network of gene-gene interactions driven by aging in the dermal fibroblast transcriptome was inferred using EDM. This network was then used to extract top-predicted candidate drivers of aging, which informs a library for further experiments. The core method for assessing the rejuvenative potential of these genes involves manipulating the expression of each library member in primary dermal fibroblasts. An imaging procedure was used to assess the physiology and morphology of each single cell, which were united in a machine learning framework to provide insight into the effects of aging on skin cells as well as to measure the rejuvenative impact of the library members using orthogonal assays. Understanding of the library's effects were then augmented on cells by analyzing functional, proteomic, and transcriptomic shifts that are affected by overexpressing each library member in older human cells.

Aspects of the present disclosure relate to a method, comprising: contacting a cell with an effective amount of a protein or a nucleic acid encoding the protein, wherein the protein is: ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof; wherein the effective amount is sufficient to induce cellular rejuvenation and/or regeneration of the cell.

Aspects of the present disclosure relate to a method, comprising: administering to a subject an effective amount of a protein or a nucleic acid encoding the protein, wherein the protein is: ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IP05, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof; wherein the effective amount is sufficient to induce cellular rejuvenation of a cell in the subject.

In some embodiments, the protein is selected from the group consisting of: ANXA2, PRRC2B, MAP4, BGN, CLTA, RPS23, AKT1, VCP, FAM129B, LMAN1, GUK1, SEC61A1, ELL2, RPN1, KIF5B, MSN, and TGOLN2. In some embodiments, the protein is selected from the group consisting of: ANXA2, PRRC2B, MAP4, BGN, CLTA, and RPS23. In some embodiments, the protein is ANXA2. In some embodiments, the protein is PRRC2B. In some embodiments, the protein is MAP4. In some embodiments, the protein is BGN. In some embodiments, the protein is CLTA. In some embodiments, the protein is RPS23.

In some embodiments, the effective amount is sufficient to reduce reactive oxygen species (ROS) abundance in the cell, compared to an untreated cell. In some embodiments, the ROS abundance in the cell is reduced by at least 1%, at least 2%, at least 3%, at least 4%, at least 5%, at least 10%, at least 15%, at least 20%, or at least 25%, compared to an untreated cell. In some embodiments, the effective amount is sufficient to increase the growth rate of the cell, compared to an untreated cell. In some embodiments, the growth rate of the cell is increased by at least 1%, at least 2%, at least 3%, at least 4%, at least 5%, at least 10%, at least 15%, at least 20%, at least 25%, at least 30%, at least 35%, or at least 40%, compared to an untreated cell.

In some embodiments, two or more proteins are selected, wherein the two or more proteins are encoded by two or more nucleic acids and the two or more nucleic acids are separated by a polycistronic element. In some embodiments, the polycistronic element is an IRES or a 2A sequence.

In some embodiments, the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years. In some embodiments, average cellular rejuvenation is defined as the product of (A) the fraction of treated, misclassified cells in a supervised two-class machine learning ensemble, wherein an untreated training population of each class exceeds 1,000 cells; and (B) the age difference between the two classes.

In some embodiments, the effective amount is sufficient to increase the signal intensity of cytoplasmic actin stained with phalloidin and measured by microscopy, the signal intensity of the Golgi apparatus/apparati stained with wheat germ agglutinin and measured by microscopy, the signal intensity of the plasma membrane stained with wheat germ agglutinin and measured by microscopy, the signal intensity of extranuclear DNA stained with Hoechst 33342 and measured by microscopy, the signal intensity of mitochondria stained with MitoTracker Deep Red and measured by microscopy, the signal intensity of the endoplasmic reticulum stained with concanavalin A and measured by microscopy, or the peripheral cellular signal intensity of RNA stained with SYTO 14 and measured by microscopy.

In some embodiments, the effective amount is sufficient to decrease cytoplasmic volume as measured by microscopy, decrease cell volume as measured by microscopy, decrease cell surface area as measured by microscopy, or decrease the signal intensity of nuclear DNA stained with Hoechst 33342 and measured by microscopy. In some embodiments, the effective amount is sufficient to increase replicative life span of the cell by at least 25%, at least 30%, at least 35%, at least 40%, at least 45%, or at least 50%. In some embodiments, the effective amount is sufficient to increase total gene expression or extracellular matrix gene expression or collagen expression levels of the cell by at least 25%, at least 30%, at least 35%, at least 40%, at least 45%, or at least 50%. In some embodiments, the effective amount is sufficient to induce an average cellular rejuvenation of at least 20 years.

In some embodiments, the cell is selected from the group consisting of: fibroblasts, hematopoietic stem cells, endothelial cells, chondrocytes, skeletal muscle stem cells, keratinocytes, mesenchymal stem cells. and corneal epithelial cells. In some embodiments, the cells are fibroblasts. In some embodiments, the fibroblasts are human dermal fibroblasts. In some embodiments, the protein is a human, canine, feline, bovine, ovine, caprine, equine, murine, porcine or pachyderm protein. In some embodiments, the protein is delivered to skin tissue layers and structures including stratum corneum, epidermis, basement membrane, dermis, hair follicles, blood vessels, and sebaceous glands or and eccrine glands.

In some embodiments, the nucleic acid comprises a heterologous promoter operably linked to an open reading frame. In some embodiments, the heterologous promoter is a constitutive promoter or an inducible promoter. In some embodiments, the regulatory sequence comprises a cell-specific promoter or a tissue-specific promoter. In some embodiments, the regulatory sequence comprises a promoter selected from the group consisting of: an hEfla promoter, an shEfla promoter (or truncated hEfla promoter), a CAG promoter (such as cytomegalovirus, chicken beta-actin intron, splice acceptor of the rabbit beta-globin gene), a CMV promoter, an hAAT promoter, a thyroid hormone-binding globulin promoter, an albumin promoter, a thyroxin-binding globulin (TBG) promoter, a hepatic control region (HCR)-ApoCII hybrid promoter, a CASI promoter, an HCR-hAAT hybrid promoter, an hAAT promoter combined with mouse albumin gene enhancer (Ealb) element, and an apolipoprotein E promoter.

In some embodiments, the nucleic acid is operably linked to a 3′ untranslated region for RNA stability and expression in mammalian cells. In some embodiments, the 3′ untranslated region comprises a WPRE sequence, a WPRE3 sequence, an SV40 late polyadenylation signal (e.g., truncated), an HBG polyadenylation signal, a rabbit beta-globin polyadenylation signal, a bovine bgpA, an ETC polyadenylation signal, or any combination thereof.

In some embodiments, the protein is delivered in a viral vector. In some embodiments, the viral vector is an AAV vector. In some embodiments, the AAV vector is derived from an AAV serotype selected from the group consisting of: AAV1, AAV2, AAV3, AAV4, AAV5, AAV6, AAV6.2, AAV7, AAV8, AAV9, AAV10, AAV11, AAV12, AAV13, AAVrh8, AAVrh10, and AAVrh32. In some embodiments, the AAV vector is derived from an AAV6 serotype. In some embodiments, the AAV vector is derived from an AAV2 serotype.

In some embodiments, the method further comprises contacting the cell with the protein or administering the protein directly to the subject. In some embodiments, the method further comprises contacting the cell with the protein or administering to the subject the nucleic acid comprising an open reading frame encoding the protein. In some embodiments, the nucleic acid is delivered in a non-viral vector or a viral vector. In some embodiments, the contacting comprises transfecting the cell.

In some embodiments, the nucleic acid comprises DNA, RNA, or a combination thereof. In some embodiments, the protein comprises a sequence that is at least 90% identical to any one of SEQ ID NOs: 1-6. In some embodiments, the nucleic acid comprises an amino acid sequence that is at least 90% identical to any one of SEQ ID NOs: 7-12.

Aspects of the present disclosure relate to a method, comprising: overexpressing in a cell a nucleic acid encoding a protein, wherein the protein is: ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IP05, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof; wherein the overexpressing produces an effective amount of the protein in the cell, and wherein the effective amount is sufficient to induce cellular rejuvenation in the cell. In some embodiments, the protein is selected from ANXA2, PRRC2B, MAP4, BGN, CLTA, RPS23, AKT1, VCP, FAM129B, LMAN1, GUK1, SEC61A1, ELL2, RPN1, KIF5B, MSN, and TGOLN2.

In some embodiments, the protein is an endogenous protein, and the method comprises activating expression or activity of the endogenous protein at a level that is higher than a baseline level. In some embodiments, the subject has been diagnosed or is at risk of being diagnosed with a skin condition. In some embodiments, the skin condition is photoaging, intrinsic aging, aging induced by repeated facial expressions, loss of tone and gravity manifested in rough texture, sagging, wrinkles, furrows, folds, or any combinations thereof.

Aspects of the present disclosure relate to a cell, comprising: an engineered nucleic acid encoding a protein, wherein the protein is: ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof. In some embodiments, the protein is selected from the group consisting of: ANXA2, PRRC2B, MAP4, BGN, CLTA, RPS23, AKT1, VCP, FAM129B, LMAN1, GUK1, SEC61A1, ELL2, RPN1, KIF5B, MSN, and TGOLN2.

In some embodiments, the protein is a recombinant purified peptide sequence. In some embodiments, the purified peptide is introduced to cells through non-covalent complexation with protein, lipid, synthetic polymeric, cell-binding ligand agents, or pore-forming agents. In some embodiments, the purified peptide is introduced to cells through chemical formulations which induce entry into cells through osmotic shock. In some embodiments, the purified peptide is introduced to cells through chemical formulations which destabilize cellular membranes.

In some embodiments, the cell is a fibroblast. In some embodiments, the fibroblast is a human dermal fibroblast. In some embodiments, the cell is a stem cell. In some embodiments, the stem cell is selected from the group consisting of: hematopoietic stem cells, skeletal muscle stem cells, and mesenchymal stem cells. In some embodiments, the stem cell is a human induced pluripotent stem cell. In some embodiments, the cell is selected from the group consisting of: endothelial cells, chondrocytes, keratinocytes, and corneal epithelial cells. In some embodiments, the cell expresses the protein at a level that is higher than a baseline level.

Aspects of the present disclosure relate to a pharmaceutical composition comprising any one of the cells described herein and a pharmaceutically-acceptable excipient and/or polymeric carrier.

Aspects of the present disclosure relate to a pharmaceutical composition comprising: a recombinant vector genome comprising one or more transgenes encoding one or more polypeptide sequences selected from ANXA2, PRRC2B, MAP4, BGN, CLTA, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, and HSPA9, wherein the vector genome is configured to express the one or more transgene at a level sufficient to induce tissue rejuvenation and/or regeneration; and a pharmaceutically-acceptable excipient and/or polymeric carrier.

Aspects of the present disclosure relate to a method, comprising administering the pharmaceutical composition described herein to skin of a subject via intraepidermal, transepidermal, intradermal, transdermal, subcutaneous, intramuscular, or topical administration. In some embodiments, the skin exhibits signs of aging. In some embodiments, the signs of aging are indicative of a skin condition selected from photoaging, intrinsic aging, aging induced by repeated facial expressions, loss of tone and gravity manifested in rough texture, sagging, wrinkles, furrows, folds, and any combinations thereof.

### Annexin A2 (ANXA2)

The present disclosure related to the identification of Annexin A2 (ANXA2) as a gene involved in the reversal of transcriptomic aging of cells, rejuvenation of cells, the reduction of reactive oxygen species (ROS) in cells, and increased growth rate in cells, such as fibroblasts. The ANXA2 gene encodes a protein that is involved in diverse cellular processes, such as cell motility.

A non-limiting example of an amino acid sequence
associated with ANXA2 is provided here as
SEQ ID NO: 1:
MSTVHEILCKLSLEGDHSTPPSAYGSVKAYTNFDAERDALNIETAIKTKG
VDEVTIVNILTNRSNAQRQDIAFAYQRRTKKELASALKSALSGHLETLIL
GLLKTPAQYDASELKASMKGLGTDEDSLIEIICSRINQELQEINRVYKEM
YKTDLEKDIISDTSGDFRKLMVALAKGRRAEDGSVIDYELIDQDARDLYD
AGVKRKGTDVPKWISIMTERSVPHLQKVFDRYKSYSPYDMLESIRKEVKG
DLENAFLNLVOCIQNKPLYFADRLYDSMKGKGTRDKVLIRIMVSRSEVDM
LKIRSEFKRKYGKSLYYYIQQDTKGDYQKALLYLCGGDD
A non-limiting example of a nucleotide sequence
the encodes SEQ ID NO: 1 is provided here as NO:
SEQ ID 7:
ATGTCTACTGTTCACGAAATCCTGTGCAAGCTCAGCTTGGAGGGTGATCAC
TCTACACCCCCAAGTGCATATGGGTCTGTCAAAGCCTATACTAACTTTGAT
GCTGAGCGGGATGCTTTGAACATTGAAACAGCCATCAAGACCAAAGGTGTG
GATGAGGTCACCATTGTCAACATTTTGACCAACCGCAGCAATGCACAGAGA
CAGGATATTGCCTTCGCCTACCAGAGAAGGACCAAAAAGGAACTTGCATCA
GCACTGAAGTCAGCCTTATCTGGCCACCTGGAGACGTTGATTTTGGGCCTA
TTGAAGACACCTGCTCAGTATGACGCTTCTGAGCTAAAAGCTTCCATGAAG
GGGCTGGGAACCGACGAGGACTCTCTCATTGAGATCATCTGCTCCAGAACC
AACCAGGAGCTGCAGGAAATTAACAGAGTCTACAAGGAAATGTACAAGACT
GATCTGGAGAAGGACATTATTTCGGACACATCTGGTGACTTCCGCAAGCTG
ATGGTTGCCCTGGCAAAGGGTAGAAGAGCAGAGGATGGCTCTGTCATTGAT
TATGAACTGATTGACCAAGATGCTCGGGATCTCTATGACGCTGGAGTGAAG
AGGAAAGGAACTGATGTTCCCAAGTGGATCAGCATCATGACCGAGCGGAGC
GTGCCCCACCTCCAGAAAGTATTTGATAGGTACAAGAGTTACAGCCCTTAT
GACATGTTGGAAAGCATCAGGAAAGAGGTTAAAGGAGACCTGGAAAATGCT
TTCCTGAACCTGGTTCAGTGCATTCAGAACAAGCCCCTGTATTTTGCTGAT
CGGCTGTATGACTCCATGAAGGGCAAGGGGACGCGAGATAAGGTCCTGATC
AGAATCATGGTCTCCCGCAGTGAAGTGGACATGTTGAAAATTAGGTCTGAA
TTCAAGAGAAAGTACGGCAAGTCCCTGTACTATTATATCCAGCAAGACACT
AAGGGCGACTACCAGAAAGCGCTGCTGTACCTGTGTGGTGGAGATGAC

### Protein Rich Coiled-Coil 2B (PRRC2B)

The present disclosure related to the identification of Protein Rich Coiled-Coil 2B (PRRC2B) as a gene involved in the reversal of transcriptomic aging of cells, rejuvenation of cells, the reduction of reactive oxygen species (ROS) in cells, and increased growth rate in cells, such as fibroblasts. The PRRC2B gene encodes a protein that is involved in diverse cellular processes, such as cell differentiation.

A non-limiting example of an amino acid sequence associated with PRRC2B is
provided here as SEQ ID NO: 2:
MSDRLGQITKGKDGKSKYSTLSLFDKYKGKSVDAIRSSVIPRHGLQSLGKVAAARRMPPPANLPSLKSENKGNDP
NIVIVPKDGTGWANKQDQQDPKSSSATASOPPESLPQPGLOKSVSNLQKPTOSISQENTNSVPGGPKSWAQLNGK
PVGHEGGLRGSSRLLSFSPEEFPTLKAAGGQDKAGKEKGVLDLSYGPGPSLRPQNVTSWREGGGRHIISATSLST
SPTELGSRNSSTGDGAPSSACTSDSKDPSLRPAQPVRKGASQFMGNVYHPPTYHDMLPAFMCSPKSSENQGTVER
GSFPLPQLRLEPRVPFRQFQMNDQDGKENRLGLSRPLRPLROLVERAPRPTIINAENLKGLDDLDADADDGWAGL
HEEVDYSEKLKFSDDEEEEEVVKDGRPKWNSWDPRRORQLSMSSADSADAKRTREEGKDWAEAVGASRVVRKAPD
PQPPPRKLHGWAPGPDYQKSSMGSMFRQQSIEDKEDKPPPROKFIQSEMSEAVERARKRREEEERRAREERLAAC
AAKLKQLDQKCKQARKAGEARKQAEKEVPWSPSAEKASPQENGPAVHKGSPEFPAQETPTTFPEEAPTVSPAVAQ
SNSSEEEAREAGSPAQEFKYQKSLPPRFOROOOOOOQEQLYKMQHWQPVYPPPSHPQRTFYPHHPQMLGFDPRWM
MMPSYMDPRITPTRTPVDFYPSALHPSGLMKPMMPQESLNGTGCRSEDQNCVPPLQERKVTPIDSPPVWSPEGYM
ALQSKGYPLPHPKSSDTLAMDMRVRNESSFSASLGRAGGVSAORDLFEERGEEYLSAFDKKAQADEDSCISSQRI
GQELLFPPQENVQDAGAPGGHTONLRCSPLEPDFVPDEKKPECGSWDVSHOPETADTAHGVERETPREGTAFNIS
SWDKNGSPNKQPSSEPEWTPEPRSSSSQHPEQTGRTRRSGPIKKPVLKALKVEDKEKELEKIKQELGEESTRLAK
EKEQSPTAEKDEDEENDASLANSSTTTLEDKGPGHATFGREATKFEEEEKPDKAWEARPPRESSDVPPMKRNNWI
FIDEEQAFGVRGQARGRGRGFREFTERGRPAGGNGSGLCGGGVLGARSIYCSSQRSGRGRGLREFARPEDCPRAK
PRRRVASETHSEGSEYEELPKRRRQRGSENGNEGSLLEREESTLKKGDCRDSWRSNKGCSEDHSGLDAKSRGPRA
FGRALPPRLSNCGYGRRTFVSKESPHWQSKSPGSSWQEYGPSDTCGSRRPTDRDYVPDSYRHPDAFGGRGFEDSR
AEDKRSFFQDEHVADSENAENRPFRRRRPPRQDKPPRFRRLRQERESLGLWGPEEEPHLLAGQWPGRPKLCSGDK
SGTVGRRSPELSYQNSSDHANEEWETASESSDFSERRERREGPGSEPDSQVDGGLSGASLGEKKELAKRSFSSQR
PVVDRQSRKLEPGGFGEKPVRPGGGDTSPRYESQQNGTPLKVKRSPDEALPGGLSGCSSGSGHSPYALERAAHAS
ADLPEASSKKAEKEAKLAAPRAGEQGEAMKQFDLNYGSAIIENCGSSPGEESEVGSMVGEGFIEVLTKKQRRLLE
EERRKKEQAVQVPVKGRGLSSRIPPRFAKKONNLCLEQGDVTVPGSSLGTEIWESSSQALPVQAPANDSWRKAVT
AFSSTETGSAEQGFKSSQGDSGVDLSAESRESSATSSORSSPYGTLKPEEMSGPGLAEPKADSHKEQAPKPSEQK
DSEQGSGQSKEHRPGPIGNERSLKNRKGSEGAERLQGAVVPPVNGVEIHVDSVLPVPPIEFGVSPKDSDFSLPPG
SASGPTGSPVVKLQDALASNAGLTQSIPILRRDHHIQRAIGLSPMSFPTADLTLKMESARKAWENSPSLPEQSSP
GGAGSGIQPPSSVGASSGVNYSSFGGVSMPPMPVASVAPSASMPGSHLPPLYLDGHVFASQPRLVPQTIPQQQSY
QQAAAAQQIPISLHTSLQAQAQLGLRGGLPVSQSQEIFSSLQPFRSQVYMHPSLSPPSTMILSGGTALKPPYSAF
PGMQPLEMVKPQSGSPYQPMSGNQALVYEGOLSQAAGLGASQMLDSQLPOLTMPLPRYGSGQQPLILPQSIQLPP
GQSLSVGAPRRIPPPGSQPPVLNTSREPSQMEMKGFHFADSKONVPSGGPVPSPQTYRPSSASPSGKPSGSAVNM
GSVQGHYVQQAKORVDEKPSLGAVKLQEAPSAASQMKRTGAIKPRAVKVEESKA
A non-limiting example of a nucleotide sequence the encodes SEQ ID NO: 2 is
provided here as SEQ ID NO: 8:
ATGTCCGATCGTTTGGGGCAAATTACCAAGGGCAAGGATGGGAAAAGCAAGTACTCGACTCTCAGCCTGTTTGAT
AAGTATAAAGGAAAATCAGTAGACGCGATTAGATCCTCAGTTATTCCTAGACATGGCTTACAGAGTCTTGGGAAA
GTTGCTGCAGCCCGGCGCATGCCACCGCCTGCAAACCTGCCAAGCTTGAAGTCTGAAAACAAAGGAAACGACCCC
AACATCGTGATAGTACCCAAGGACGGGACGGGATGGGCAAACAAGCAGGATCAGCAAGACCCAAAGAGTTCCAGT
GCGACGGCCTCTCAGCCGCCGGAGTCGCTGCCGCAGCCGGGTTTGCAGAAATCTGTCTCCAATTTGCAGAAACCG
ACACAGTCAATCAGTCAGGAGAATACAAATTCAGTGCCAGGTGGACCAAAGTCATGGGCACAGCTGAATGGAAAG
CCAGTAGGACACGAAGGTGGTTTAAGGGGCTCAAGCCGACTGTTATCCTTCTCTCCCGAGGAATTTCCGACGCTG
AAAGCAGCTGGAGGGCAGGACAAGGCTGGCAAAGAAAAGGGCGTCTTAGATCTGTCGTATGGGCCAGGACCAAGC
CTCCGCCCTCAGAATGTGACAAGCTGGAGGGAGGGCGGTGGGCGACACATAATTTCTGCCACGTCTCTGAGCACC
TCCCCAACTGAGCTGGGCAGCAGGAACTCGAGTACGGGAGATGGAGCCCCCTCCTCGGCATGTACCAGCGATTCT
AAGGACCCCTCTCTCCGCCCGGCTCAGCCTGTCCGAAAAGGGGCTTCACAGTTCATGGGAAATGTATACCACCCA
CCTACATACCATGACATGCTTCCTGCTTTTATGTGTTCGCCGAAGTCATCAGAAAACCAGGGTACAGTGGAACGA
GGCTCTTTTCCCCTTCCTCAGCTCCGCCTTGAACCTCGAGTTCCTTTTAGACAGTTCCAGATGAATGACCAAGAC
GGAAAAGAAAACAGGCTGGGATTGTCTCGCCCACTCCGCCCACTAAGGCAGCTGGTGGAGCGGGCACCACGGCCC
ACCATTATCAATGCGGAAAACCTGAAGGGCCTTGACGATCTGGACGCCGATGCCGATGATGGCTGGGCAGGCCTC
CATGAAGAAGTGGACTATTCTGAGAAACTGAAGTTCAGTGATGATGAAGAGGAGGAAGAAGTIGTGAAGGACGGC
AGGCCAAAGTGGAACAGTTGGGACCCTAGGAGGCAGCGGCAGTTGTCAATGAGCTCTGCAGACAGTGCGGACGCT
AAGCGGACTCGAGAGGAAGGGAAGGACTGGGCTGAAGCAGTGGGTGCGTCCCGTGTGGTCCGAAAGGCGCCAGAC
CCTCAGCCACCGCCCAGGAAGCTTCATGGCTGGGCACCAGGCCCTGACTACCAGAAGTCATCAATGGGCAGCATG
TTCCGGCAACAGTCCATCGAGGACAAGGAGGACAAGCCCCCACCAAGGCAGAAGTTCATTCAGTCAGAGATGTCC
GAGGCGGTGGAGCGAGCCCGAAAGCGCCGGGAAGAAGAGGAGCGCCGAGCCCGGGAGGAGAGGCTGGCCGCCTGT
GCTGCCAAACTCAAGCAGCTGGACCAGAAGTGTAAGCAGGCACGAAAGGCAGGTGAGGCCCGGAAGCAGGCAGAG
AAGGAAGTGCCCTGGTCTCCAAGTGCTGAGAAGGCATCTCCCCAGGAAAACGGCCCTGCTGTCCACAAAGGCTCC
CCAGAATTCCCTGCCCAAGAGACCCCCACCACATTCCCAGAAGAGGCACCCACAGTGTCCCCAGCAGTGGCACAG
AGCAACAGCAGTGAGGAAGAGGCCAGAGAGGCTGGGTCCCCTGCACAGGAGTTCAAGTATCAGAAGTCCCTTCCT
CCCCGATTCCAGCGCCAGCAGCAGCAACAACAGCAGGAGCAGCTGTACAAGATGCAGCACTGGCAGCCGGTGTAC
CCCCCGCCGTCCCACCCCCAGCGCACCTTTTACCCACACCACCCCCAGATGTTGGGCTTCGATCCCAGGTGGATG
ATGATGCCTTCCTACATGGACCCACGTATCACGCCCACTCGGACCCCGGTGGACTTCTACCCCTCCGCCCTGCAT
CCCTCAGGACTGATGAAGCCCATGATGCCCCAGGAGTCCCTCAATGGGACAGGCTGTCGCTCTGAGGATCAGAAC
TGTGTGCCCCCACTCCAAGAAAGAAAAGTGACCCCCATCGACTCACCCCCTGTGTGGAGCCCAGAGGGCTACATG
GCACTGCAGAGCAAGGGCTACCCGCTCCCGCACCCGAAGTCGAGTGACACCTTGGCTATGGACATGCGTGTCAGG
AATGAAAGCTCTTTCTCTGCCTCACTCGGAAGGGCAGGGGGCGTAAGTGCTCAGCGCGATCTCTTTGAGGAGAGA
GGGGAGGAGTACTTGAGTGCTTTTGACAAGAAGGCCCAAGCAGACTTTGACAGCTGTATCTCTTCTCAAAGAATA
GGCCAGGAGCTTTTGTTTCCACCCCAAGAAAATGTTCAGGATGCAGGTGCTCCTGGGGGTCACACCCAAAACCTC
AGGTGTTCCCCATTGGAGCCTGACTTTGTCCCAGATGAGAAAAAGCCAGAGTGTGGCAGTTGGGATGTTAGCCAC
CAGCCAGAGACCGCTGACACAGCCCATGGTGTTGAGCGGGAGACACCCCGGGAGGGGACGGCCTTTAACATCTCC
TCCTGGGACAAGAACGGGAGCCCCAACAAACAGCCATCCTCGGAGCCTGAATGGACTCCCGAGCCCCGGAGCTCC
AGCAGCCAGCACCCGGAGCAGACGGGCAGGACCCGGAGGTCGGGACCCATCAAGAAACCAGTCCTGAAAGCCCTC
AAGGTGGAAGACAAGGAGAAGGAGCTTGAGAAGATTAAGCAGGAGCTAGGGGAGGAGAGTACCCGGCTGGCCAAG
GAGAAGGAGCAGAGCCCCACGGCAGAAAAGGATGAGGACGAAGAGAACGATGCCTCTCTGGCCAACTCCTCCACC
ACCACTTTGGAGGACAAAGGCCCTGGCCATGCCACTTTTGGCCGCGAGGCCACCAAATTTGAAGAGGAGGAGAAA
CCTGACAAGGCCTGGGAAGCCAGACCCCCACGAGAGTCCAGCGATGTTCCCCCCATGAAGAGAAATAACTGGATC
TTTATTGATGAGGAGCAAGCCTTTGGGGTCAGAGGACAGGCCCGGGGCCGGGGCCGTGGTTTCAGAGAGTTCACT
TTTCGTGGTCGGCCTGCTGGCGGAAATGGGAGCGGCCTCTGTGGTGGGGGGGTCCTGGGGGCCCGCAGCATCTAC
TGCAGCAGTCAGCGCAGCGGCCGTGGCCGGGGCCTGCGAGAGTTTGCGCGGCCAGAGGACTGCCCCAGAGCCAAG
CCCCGACGGAGAGTTGCCAGTGAGACCCATAGCGAGGGCTCAGAGTATGAAGAACTTCCCAAGCGCCGCCGGCAG
AGGGGCTCCGAGAACGGGAATGAAGGCTCGCTCCTGGAGAGGGAGGAGAGCACCTTGAAGAAGGGCGACTGCAGA
GATTCTTGGCGGTCCAACAAGGGGTGCTCTGAGGACCACAGCGGTCTAGATGCCAAGAGCCGAGGCCCTCGGGCC
TTTGGGCGAGCCCTCCCTCCCCGGCTGAGCAATTGCGGGTATGGACGGAGAACCTTCGTCTCCAAAGAGTCACCC
CACTGGCAGAGCAAAAGTCCAGGCAGCTCTTGGCAGGAATATGGCCCTTCCGACACATGCGGATCCCGGCGACCT
ACAGACAGAGACTATGTCCCAGATTCCTACAGACACCCTGACGCATTTGGTGGCCGGGGCTTTGAGGACAGCCGC
GCGGAGGACAAGAGATCCTTCTTCCAAGATGAACACGTGGCAGATTCTGAAAATGCAGAGAACCGGCCCTTCAGG
AGAAGGCGCCCCCCACGCCAAGATAAGCCCCCTCGATTCCGGCGCCTCCGGCAAGAGCGGGAGTCCCTGGGCCTG
TGGGGACCCGAGGAGGAGCCCCACCTGCTGGCAGGTCAGTGGCCAGGCAGGCCCAAACTGTGTTCTGGGGACAAG
AGTGGCACTGTGGGCCGCAGGTCCCCTGAGCTCTCCTACCAGAACTCCTCCGATCACGCCAATGAGGAGTGGGAG
ACGGCCTCCGAAAGCAGCGACTTCAGCGAGCGGCGGGAGCGGCGGGAAGGCCCTGGGTCCGAGCCCGACTCCCAG
GTGGATGGTGGCCTGTCGGGGGCTAGTTTGGGTGAGAAGAAGGAGCTGGCCAAGAGGAGCTTCTCCAGTCAGAGA
CCCGTGGTTGACAGACAGAGCCGAAAGCTGGAGCCGGGAGGGTTTGGGGAGAAGCCCGTTAGGCCAGGTGGTGGT
GACACCTCCCCTCGCTATGAGAGCCAACAGAATGGGACGCCTTTGAAAGTGAAAAGATCCCCAGACGAGGCCTTG
CCTGGAGGTCTTAGTGGCTGCAGCAGTGGGAGTGGCCACTCCCCCTATGCCCTGGAGCGGGCAGCCCATGCCAGT
GCTGACCTTCCCGAAGCCTCCAGTAAAAAGGCAGAGAAGGAGGCCAAGTTGGCTGCTCCGAGGGCAGGTGAACAG
GGAGAGGCCATGAAACAGTTTGACCTGAACTATGGAAGTGCCATCATTGAAAATTGCGGGTCCAGCCCCGGGGAG
GAGAGTGAGGTGGGTTCTATGGTGGGCGAAGGCTTCATCGAAGTCCTGACCAAGAAGCAGCGCCGCCTGCTGGAG
GAAGAGAGAAGAAAGAAGGAGCAGGCCGTGCAGGTGCCTGTCAAAGGTCGAGGCCTTTCCTCCCGTATTCCTCCT
CGATTTGCAAAAAAGCAGAACAACTTATGTCTGGAGCAAGGTGACGTGACCGTGCCTGGCAGCAGCCTGGGCACT
GAGATCTGGGAGAGCAGCAGCCAGGCTCTCCCTGTGCAGGCCCCAGCCAACGACTCCTGGAGGAAAGCTGTCACT
GCCTTCAGCAGCACCGAGACTGGCTCTGCGGAGCAGGGTTTTAAGAGCAGCCAGGGAGATAGTGGCGTTGACTTG
AGTGCCGAGTCTCGGGAGTCGTCTGCGACCTCCTCGCAGCGCAGCTCCCCATATGGGACTCTGAAGCCAGAGGAG
ATGAGCGGGCCCGGCCTGGCGGAACCCAAGGCCGACAGCCACAAGGAGCAGGCTCCAAAGCCATCTGAGCAGAAG
GATTCAGAACAAGGCTCTGGACAGAGCAAGGAGCACAGACCAGGACCCATCGGCAACGAGCGTTCTCTGAAAAAC
AGAAAGGGCTCGGAGGGGGCCGAGCGGCTGCAAGGGGCTGTCGTCCCGCCTGTTAACGGGGTGGAGATTCACGTG
GACTCCGTGCTGCCTGTGCCACCCATTGAATTTGGAGTCAGTCCAAAAGACTCCGATTTCAGCTTGCCACCTGGT
TCTGCCTCTGGTCCTACTGGGAGTCCAGTTGTTAAACTTCAGGATGCCTTGGCCAGTAATGCAGGGTTAACACAG
AGTATCCCCATCCTGCGGCGGGACCATCACATCCAGAGGGCCATCGGTCTCTCCCCAATGTCCTTCCCCACCGCC
GACCTTACTCTGAAGATGGAGTCTGCGCGCAAGGCTTGGGAAAACTCCCCCAGTTTGCCGGAGCAGAGCTCTCCA
GGCGGCGCTGGCTCAGGCATCCAGCCTCCATCCTCTGTGGGTGCCTCCAGCGGGGTCAACTACAGCTCCTTCGGT
GGAGTGTCCATGCCACCCATGCCTGTGGCCTCTGTAGCACCTTCTGCTTCTATGCCAGGCAGCCACCTCCCGCCC
CTGTACCTGGATGGCCATGTGTTTGCAAGTCAGCCCCGGCTGGTTCCTCAAACGATACCTCAGCAGCAGAGTTAC
CAACAGGCCGCCGCTGCCCAGCAGATCCCGATCTCCCTTCACACATCTCTGCAGGCACAAGCTCAGCTTGGACTG
AGGGGTGGGCTTCCTGTGTCCCAGTCCCAGGAGATCTTCAGCTCCTTGCAGCCCTTCAGATCTCAGGTGTACATG
CACCCCAGCCTGTCACCGCCCAGCACCATGATCCTCTCTGGGGGCACAGCCTTGAAGCCTCCATACTCGGCGTTC
CCAGGCATGCAGCCCTTGGAGATGGTGAAGCCGCAGTCTGGCTCACCCTACCAGCCCATGAGCGGGAACCAAGCC
CTGGTCTACGAGGGCCAGCTCAGCCAGGCTGCTGGCCTGGGTGCCTCCCAGATGTTGGACTCCCAGCTCCCACAG
CTGACCATGCCACTGCCTCGGTACGGCTCCGGGCAGCAGCCACTGATCCTGCCCCAGTCTATTCAGCTGCCACCT
GGGCAGAGCCTCTCCGTTGGGGCCCCCCGAAGGATTCCTCCGCCCGGGTCCCAGCCGCCAGTCCTGAACACCAGC
AGAGAGCCCTCTCAGATGGAGATGAAAGGCTTCCACTTTGCCGACAGTAAACAGAATGTCCCTTCAGGAGGCCCC
GTGCCATCGCCACAGACCTACAGGCCTAGCTCTGCTAGCCCCAGTGGGAAGCCCTCTGGATCAGCAGTTAACATG
GGCTCTGTGCAGGGACACTACGTGCAACAGGCAAAACAACGAGTGGATGAGAAACCCAGCCTGGGAGCCGTGAAG
CTGCAGGAGGCCCCCTCGGCTGCCTCCCAGATGAAGCGAACCGGAGCGATCAAGCCTCGGGCTGTCAAAGTGGAG
GAGAGTAAGGCC

### Microtubule Associated Protein 4 (MAP4)

The present disclosure related to the identification of Microtubule Associated Protein 4 (MAP4) as a gene involved in the reversal of transcriptomic aging of cells, rejuvenation of cells, the reduction of reactive oxygen species (ROS) in cells, and increased growth rate in cells, such as fibroblasts. The MAP4 gene encodes a protein that is involved in diverse cellular processes, such as cell motility.

A non-limiting example of an amino acid sequence
associated with MAP4 is provided here as SEQ ID
NO: 3:
MADLSLADALTEPSPDIEGEIKRDFIATLEAEAFDDVVGETVGKTDYIP
LLDVDEKTGNSESKKKPCSETSQIEDTPSSKPTLLANGGHGVEGSDTTG
SPTEFLEEKMAYQEYPNSQNWPEDTNFCFQPEQVVDPIQTDPFKMYHDD
DLADLVFPSSATADTSIFAGQNDPLKDSYGMSPCNTAVVPQGWSVEALN
SPHSESFVSPEAVAEPPQPTAVPLELAKEIEMASEERPPAQALEIMMGL
KTTDMAPSKETEMALAKDMALATKTEVALAKDMESPTKLDVTLAKDMQP
SMESDMALVKDMELPTEKEVALVKDVRWPTETDVSSAKNVVLPTETEVA
PAKDVTLLKETERASPIKMDLAPSKDMGPPKENKKETERASPIKMDLAP
SKDMGPPKENKIVPAKDLVLLSEIEVAQANDIISSTEISSAEKVALSSE
TEVALARDMTLPPETNVILTKDKALPLEAEVAPVKDMAQLPETEIAPAK
DVAPSTVKEVGLLKDMSPLSETEMALGKDVTPPPETEVVLIKNVCLPPE
MEVALTEDQVPALKTEAPLAKDGVLTLANNVTPAKDVPPLSETEATPVP
IKDMEIAQTQKGISEDSHLESLQDVGQSAAPTFMISPETVTGTGKKCSL
PAEEDSVLEKLGERKPCNSQPSELSSETSGIARPEEGRPVVSGTGNDIT
TPPNKELPPSPEKKTKPLATTQPAKTSTSKAKTQPTSLPKQPAPTTIGG
LNKKPMSLASGLVPAAPPKRPAVASARPSILPSKDVKPKPIADAKAPEK
RASPSKPASAPASRSGSKSTQTVAKTTTAAAVASTGPSSRSPSTLLPKK
PTAIKTEGKPAEVKKMTAKSVPADLSRPKSTSTSSMKKITTLSGTAPAA
GVVPSRVKATPMPSRPSTTPFIDKKPTSAKPSSTTPRLSRLATNTSAPD
LKNVRSKVGSTENIKHQPGGGRAKVEKKTEAAATTRKPESNAVTKTAGP
IASAQKQPAGKVQIVSKKVSYSHIQSKCGSKDNIKHVPGGGNVQIONKK
VDISKVSSKCGSKANIKHKPGGGDVKIESQKLNFKEKAQAKVGSLDNVG
HLPAGGAVKTEGGGSEAPLCPGPPAGEEPAISEAAPEAGAPTSASGLNG
HPTLSGGGDQREAQTLDSQIQETSI
A non-limiting example of a nucleotide sequence
the encodes SEQ ID NO: 3 is provided here as SEQ
ID NO: 9:
ATGGCCGACCTGAGCCTGGCTGACGCCCTGACCGAGCCCAGCCCCGACA
TTGAGGGCGAGATCAAGAGAGACTTCATCGCCACATTAGAGGCCGAGGC
CTTCGACGACGTGGTGGGCGAGACAGTGGGCAAGACAGACTACATCCCC
CTGCTGGACGTGGACGAGAAGACCGGCAACAGCGAATCCAAGAAGAAGC
CCTGCAGCGAGACAAGCCAGATCGAGGATACCCCCAGCAGCAAGCCTAC
ACTGCTGGCCAATGGCGGCCACGGAGTGGAGGGCAGCGATACCACAGGA
AGCCCCACAGAGTTCCTGGAGGAGAAGATGGCCTACCAGGAGTACCCCA
ACAGCCAGAACTGGCCCGAGGACACCAACTTCTGCTTCCAGCCCGAACA
GGTGGTGGACCCCATCCAGACCGACCCCTTCAAGATGTACCACGACGAC
GACCTGGCCGATCTGGTGTTTCCCAGCTCTGCCACCGCCGACACCAGCA
TCTTTGCCGGCCAGAACGACCCCCTTAAGGACAGCTACGGCATGTCTCC
CTGCAACACCGCCGTGGTTCCTCAGGGCTGGAGCGTGGAAGCCCTGAAT
AGCCCTCACAGCGAGTCTTTCGTTAGCCCCGAGGCCGTTGCTGAACCAC
CTCAGCCCACAGCCGTGCCACTGGAGCTTGCCAAAGAAATCGAGATGGC
CAGCGAAGAAAGGCCTCCTGCCCAGGCCCTGGAGATCATGATGGGCCTG
AAGACCACCGACATGGCCCCAAGCAAGGAGACTGAGATGGCTCTGGCCA
AGGACATGGCCCTGGCCACAAAGACCGAGGTGGCCCTGGCCAAGGACAT
GGAGTCCCCCACCAAGCTGGACGTGACACTGGCCAAAGACATGCAGCCA
AGCATGGAGAGCGACATGGCCCTGGTGAAGGATATGGAGCTGCCTACCG
AGAAGGAGGTGGCTCTGGTTAAGGACGTGAGGTGGCCCACCGAAACCGA
CGTGAGCAGCGCCAAGAACGTGGTGCTGCCAACCGAGACTGAAGTGGCC
CCCGCTAAGGATGTGACCCTGCTGAAGGAGACAGAGAGAGCCAGCCCTA
TCAAGATGGACCTGGCCCCTTCCAAGGACATGGGCCCCCCCAAGGAGAA
TAAGAAGGAAACCGAGCGGGCCAGCCCCATCAAGATGGATCTGGCCCCC
TCCAAGGATATGGGCCCTCCTAAGGAGAACAAGATCGTGCCCGCCAAGG
ACCTGGTGCTGCTGAGCGAGATCGAGGTGGCCCAGGCCAACGACATCAT
CTCTAGCACCGAGATTAGCAGCGCCGAGAAAGTGGCCCTGAGCTCCGAG
ACAGAAGTGGCCCTGGCCCGGGACATGACACTGCCTCCCGAAACAAACG
TGATCCTGACCAAGGACAAGGCCCTCCCTCTGGAGGCCGAGGTGGCCCC
TGTTAAGGATATGGCCCAGCTGCCTGAGACAGAGATCGCACCAGCCAAG
GACGTGGCTCCCTCTACAGTGAAGGAGGTGGGCCTGCTGAAGGACATGT
CTCCCCTGAGCGAAACCGAGATGGCCCTGGGCAAAGACGTGACCCCCCC
ACCCGAGACAGAGGTGGTGCTGATCAAGAACGTGTGCCTGCCACCAGAG
ATGGAGGTTGCCCTGACAGAGGACCAGGTGCCTGCCCTGAAAACCGAAG
CTCCCCTTGCCAAGGATGGCGTTCTGACCCTCGCTAATAACGTGACTCC
TGCTAAGGACGTGCCCCCCCTGTCCGAGACTGAGGCCACCCCCGTGCCC
ATCAAAGATATGGAGATCGCCCAGACCCAGAAGGGCATTAGCGAGGACA
GCCACCTGGAATCTCTGCAGGACGTGGGCCAGAGCGCCGCCCCTACCTT
CATGATCTCTCCCGAGACTGTGACCGGAACCGGCAAGAAATGCAGCCTG
CCTGCCGAGGAAGACAGCGTGCTCGAGAAGCTGGGCGAGAGAAAGCCTT
GCAACTCCCAGCCCAGCGAGCTGTCTAGCGAAACAAGCGGAATTGCCCG
GCCTGAGGAGGGAAGACCCGTGGTGTCTGGCACCGGCAACGATATCACC
ACCCCTCCCAACAAGGAGCTGCCCCCCTCTCCTGAGAAGAAGACCAAGC
CCCTGGCTACAACCCAGCCCGCCAAGACCTCTACCAGCAAGGCCAAAAC
CCAACCCACCAGCCTGCCCAAGCAGCCTGCTCCCACAACAATCGGCGGA
CTTAATAAGAAGCCCATGAGCCTTGCCAGCGGCCTGGTGCCAGCCGCTC
CTCCTAAAAGACCTGCCGTGGCCTCTGCCAGACCCTCCATTCTGCCCAG
CAAGGATGTGAAGCCTAAGCCCATCGCCGATGCCAAGGCTCCCGAGAAG
AGAGCTAGCCCTAGCAAGCCAGCCAGCGCTCCCGCCAGCAGAAGCGGAA
GCAAGAGCACACAGACAGTGGCTAAGACAACAACCGCTGCCGCAGTGGC
TTCTACCGGCCCCTCCTCTAGATCCCCCAGCACACTGCTGCCCAAAAAG
CCCACCGCCATCAAGACAGAGGGAAAGCCCGCTGAGGTGAAGAAGATGA
CCGCCAAGAGCGTGCCCGCCGACCTTTCCAGGCCCAAGTCTACCTCCAC
AAGCTCTATGAAGAAGACAACCACACTGTCTGGAACCGCCCCAGCCGCC
GGCGTGGTGCCTAGCAGAGTGAAGGCCACCCCTATGCCCAGCAGACCTT
CTACAACCCCTTTCATCGACAAGAAGCCCACAAGCGCCAAGCCCTCCAG
CACCACCCCCAGACTGAGCAGACTGGCCACCAATACCAGCGCCCCTGAC
CTGAAGAATGTGCGGAGCAAAGTGGGCTCCACCGAGAACATCAAGCACC
AGCCCGGCGGCGGCAGAGCCAAGGTGGAGAAGAAAACAGAAGCCGCCGC
CACAACCAGAAAACCCGAGTCTAACGCTGTGACCAAAACAGCCGGCCCC
ATTGCCAGCGCCCAGAAGCAGCCAGCCGGCAAGGTGCAGATCGTCAGCA
AGAAGGTGTCCTACAGCCACATCCAGAGCAAGTGCGGCAGCAAGGACAA
TATCAAGCACGTGCCAGGCGGCGGCAACGTGCAGATCCAGAACAAGAAG
GTGGACATCAGCAAGGTGAGCTCCAAGTGCGGCTCCAAGGCCAACATCA
AGCACAAGCCTGGCGGCGGCGACGTGAAGATCGAGAGCCAGAAGCTCAA
CTTCAAGGAGAAGGCCCAGGCTAAGGTGGGCAGCCTTGATAACGTTGGC
CACTTACCTGCCGGCGGCGCTGTGAAGACCGAAGGCGGAGGATCTGAGG
CCCCTCTGTGTCCTGGACCTCCTGCTGGAGAGGAGCCTGCTATTTCTGA
AGCTGCTCCTGAGGCTGGCGCTCCTACATCTGCATCTGGCCTGAACGGA
CACCCCACCCTGAGCGGCGGCGGAGATCAGAGAGAAGCTCAGACCCTGG
ACTCTCAGATCCAGGAGACAAGCATC

### Biglycan (BGN)

The present disclosure related to the identification of Biglycan (BGN) as a gene involved in the reversal of transcriptomic aging of cells, rejuvenation of cells, the reduction of reactive oxygen species (ROS) in cells, and increased growth rate in cells, such as fibroblasts. The BGN gene encodes a protein that is involved in diverse cellular processes, such as cell growth.

A non-limiting example of an amino acid sequence
associated with BGN is provided here as SEQ ID
NO: 4:
MWPLWRLVSLLALSQALPFEQRGFWDFTLDDGPFMMNDEEASGADTSGV
LDPDSVTPTYSAMCPFGCHCHLRVVQCSDLGLKSVPKEISPDTTLLDLQ
NNDISELRKDDFKGLQHLYALVLVNNKISKIHEKAFSPLRKLQKLYISK
NHLVEIPPNLPSSLVELRIHDNRIRKVPKGVFSGLRNMNCIEMGGNPLE
NSGFEPGAFDGLKLNYLRISEAKLTGIPKDLPETLNELHLDHNKIQAIE
LEDLLRYSKLYRLGLGHNQIRMIENGSLSFLPTLRELHLDNNKLARVPS
GLPDLKLLQVVYLHSNNITKVGVNDFCPMGFGVKRAYYNGISLFNNPVP
YWEVQPATFRCVTDRLAIQFGNYKK
A non-limiting example of a nucleotide sequence
the encodes SEQ ID NO: 4 is provided here as SEQ
ID NO: 10:
ATGTGGCCCCTGTGGCGCCTCGTGTCTCTGCTGGCCCTGAGCCAGGCCC
TGCCCTTTGAGCAGAGAGGCTTCTGGGACTTCACCCTGGACGATGGGCC
ATTCATGATGAACGATGAGGAAGCTTCGGGCGCTGACACCTCAGGCGTC
CTGGACCCGGACTCTGTCACACCCACCTACAGCGCCATGTGTCCTTTCG
GCTGCCACTGCCACCTGCGGGTGGTTCAGTGCTCCGACCTGGGTCTGAA
GTCTGTGCCCAAAGAGATCTCCCCTGACACCACGCTGCTGGACCTGCAG
AACAACGACATCTCCGAGCTCCGCAAGGATGACTTCAAGGGTCTCCAGC
ACCTCTACGCCCTCGTCCTGGTGAACAACAAGATCTCCAAGATCCATGA
GAAGGCCTTCAGCCCACTGCGGAAGCTGCAGAAGCTCTACATCTCCAAG
AACCACCTGGTGGAGATCCCGCCCAACCTACCCAGCTCCCTGGTGGAGC
TCCGCATCCACGACAACCGCATCCGCAAGGTGCCCAAGGGAGTGTTCAG
CGGGCTCCGGAACATGAACTGCATCGAGATGGGCGGGAACCCACTGGAG
AACAGTGGCTTTGAACCTGGAGCCTTCGATGGCCTGAAGCTCAACTACC
TGCGCATCTCAGAGGCCAAGCTGACTGGCATCCCCAAAGACCTCCCTGA
GACCCTGAATGAACTCCACCTAGACCACAACAAAATCCAGGCCATCGAA
CTGGAGGACCTGCTTCGCTACTCCAAGCTGTACAGGCTGGGCCTAGGCC
ACAACCAGATCAGGATGATCGAGAACGGGAGCCTGAGCTTCCTGCCCAC
CCTCCGGGAGCTCCACTTGGACAACAACAAGTTGGCCAGGGTGCCCTCA
GGGCTCCCAGACCTCAAGCTCCTCCAGGTGGTCTATCTGCACTCCAACA
ACATCACCAAAGTGGGTGTCAACGACTTCTGTCCCATGGGCTTCGGGGT
GAAGCGGGCCTACTACAACGGCATCAGCCTCTTCAACAACCCCGTGCCC
TACTGGGAGGTGCAGCCGGCCACTTTCCGCTGCGTCACTGACCGCCTGG
CCATCCAGTTTGGCAACTACAAAAAG

### Clathrin Light Chain A (CLTA)

The present disclosure related to the identification of Clathrin Light Chain A (CLTA) as a gene involved in the reversal of transcriptomic aging of cells, rejuvenation of cells, the reduction of reactive oxygen species (ROS) in cells, and increased growth rate in cells, such as fibroblasts. The CLTA gene encodes a protein that is involved in diverse cellular processes, such as endocytosis.

A non-limiting example of an amino acid sequence
associated with CLTA is provided here as SEQ ID
NO: 5:
MAELDPFGAPAGAPGGPALGNGVAGAGEEDPAAAFLAQQESEIAGIEND
EAFAILDGGAPGPQPHGEPPGGPDAVDGVMNGEYYQESNGPTDSYAAIS
QVDRLQSEPESIRKWREEQMERLEALDANSRKQEAEWKEKAIKELEEWY
ARQDEQLQKTKANNRVADEAFYKQPFADVIGYVTNINHPCYSLEQAAEE
AFVNDIDESSPGTEWERVARLCDENPKSSKQAKDVSRMRSVLISLKQAP
LVH
A non-limiting example of a nucleotide sequence
the encodes SEQ ID NO: 5 is provided here as SEQ
ID NO: 11:
ATGGCTGAATTAGATCCTTTCGGAGCTCCTGCTGGCGCTCCTGGAGGTC
CTGCTTTAGGAAATGGAGTTGCTGGAGCCGGAGAAGAAGATCCTGCTGC
GGCCTTTTTAGCTCAGCAAGAGAGCGAGATTGCTGGCATTGAGAATGAC
GAGGCCTTTGCTATTCTTGACGGAGGAGCTCCTGGGCCTCAACCTCATG
GAGAACCTCCTGGGGGTCCGGATGCTGTTGATGGAGTAATGAATGGTGA
ATACTACCAGGAAAGTAATGGTCCAACAGACAGTTATGCAGCTATTTCA
CAAGTGGATCGACTGCAGTCAGAGCCTGAAAGTATCCGTAAATGGAGAG
AAGAACAAATGGAACGCTTGGAAGCCCTTGATGCCAATTCTCGGAAGCA
AGAAGCAGAGTGGAAAGAAAAGGCAATAAAGGAGCTAGAAGAATGGTAT
GCAAGACAGGACGAGCAGCTACAGAAAACAAAAGCAAACAACAGGGTGG
CCGATGAAGCTTTCTACAAACAACCCTTCGCTGACGTGATTGGTTATGT
CACAAACATAAACCATCCTTGCTACAGCCTAGAACAGGCAGCAGAAGAA
GCCTTTGTAAATGACATTGACGAGTCGTCCCCAGGCACTGAGTGGGAAC
GGGTGGCCAGGCTTTGTGACTTTAACCCTAAGTCTAGCAAGCAGGCCAA
AGATGTCTCCCGCATGCGCTCAGTCCTCATCTCCCTCAAGCAAGCCCCG
CTGGTGCAC

### Ribosomal Protein S23 (RPS23)

The present disclosure related to the identification of Ribosomal Protein S23 (RPS23) as a gene involved in the reversal of transcriptomic aging of cells, rejuvenation of cells, the reduction of reactive oxygen species (ROS) in cells, and increased growth rate in cells, such as fibroblasts. The RPS23 gene encodes a protein that is involved in diverse cellular processes, such as protein synthesis and cell growth.

A non-limiting example of an amino acid sequence
associated with RPS23 is provided here as SEQ ID
NO: 6:
MGKCRGLRTARKLRSHRRDQKWHDKQYKKAHLGTALKANPFGGASHAKG
IVLEKVGVEAKQPNSAIRKCVRVQLIKNGKKITAFVPNDGCLNFIEEND
EVLVAGFGRKGHAVGDIPGVRFKVVKVANVSLLALYKGKKERPRS
A non-limiting example of a nucleotide sequence
the encodes SEQ ID NO: 6 is provided here as SEQ
ID NO: 12:
ATGGGCAAGTGTCGTGGACTTCGTACTGCTAGGAAGCTCCGTAGTCACC
GACGAGACCAGAAGTGGCATGATAAACAGTATAAGAAAGCTCATTTGGG
CACAGCCCTAAAGGCCAACCCTTTTGGAGGTGCTTCTCATGCAAAAGGA
ATCGTGCTGGAAAAAGTAGGAGTTGAAGCCAAACAGCCAAATTCTGCCA
TTAGGAAGTGTGTAAGGGTCCAGCTGATCAAGAATGGCAAGAAAATCAC
AGCCTTTGTACCCAATGACGGTTGCTTGAACTTTATTGAGGAAAATGAT
GAAGTTCTGGTTGCTGGATTTGGTCGCAAAGGTCATGCTGTTGGTGATA
TTCCTGGAGTCCGCTTTAAGGTTGTCAAAGTAGCCAATGTTTCTCTTTT
GGCCCTATACAAAGGCAAGAAGGAAAGACCAAGATCA

### Proteins

The cells of the present disclosure, in some embodiments, comprise proteins encoded by engineered nucleic acids. The term “protein” encompasses full length functional ANXA2, PRRC2B, MAP4, BGN, CLTA, RPS23, and other proteins as well as full-length or truncated functional variants of a protein, unless stated otherwise. Thus, the term “protein” encompasses full length functional ANXA2, PRRC2B, MAP4, BGN, CLTA, RPS23 proteins as well as full-length or truncated functional variants of the ANXA2, PRRC2B, MAP4, BGN, CLTA, RPS23 protein, unless stated otherwise. Thus, in some embodiments, an ANXA2 protein comprises the sequence of SEQ ID NO: 1 or is encoded by a nucleic acid comprising a protein coding sequence of SEQ ID NO: 2. In other embodiments, an ANXA2 protein comprises a sequence having at least 70% identity to the sequence of SEQ ID NO: 1 or is encoded by a nucleic acid comprising a sequence having at least 70% identity to a protein coding sequence of SEQ ID NO: 7. In some embodiments, a PRRC2B protein comprises the sequence of SEQ ID NO: 2. In some embodiments, a MAP4 protein comprises the sequence of SEQ ID NO: 3. In some embodiments, a BGN protein comprises the sequence of SEQ ID NO: 4. In some embodiments, a CLTA protein comprises the sequence of SEQ ID NO: 5. In some embodiments, an RPS23 protein comprises the sequence of SEQ ID NO: 6.

### Functional Variants

The term “identity” refers to a relationship between the sequences of two or more peptides, as determined by comparing the sequences. Identity also refers to the degree of sequence relatedness between or among sequences as determined by the number of matches between strings of two or more amino acid residues. Identity measures the percent of identical matches between the smaller of two or more sequences with gap alignments (if any) addressed by a particular mathematical model or computer program. Identity of related peptides can be readily calculated by known methods. “Percent (%) identity” as it applies to peptide sequences is defined as the percentage of amino acid residues of a first sequence that is identical with the amino acid residues of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation.

In some embodiments, an ANXA2 protein comprises a sequence having at least 70% identity to the sequence of SEQ ID NO: 1 and maintains the functions described herein (e.g., capable of inducing cellular rejuvenation of a cell, for example, reducing levels of reactive oxygen species in a cell, and/or increasing cellular growth rate). For example, a functional ANXA2 protein may comprise a sequence having at least 75%, at least 80%, at least 85%, at least 90%, at least 95%, at least 96%, at least 97%, at least 98%, or at least 99% identity to the sequence of SEQ ID NO: 1.

In some embodiments, a functional ANXA2 protein is encoded by a nucleic acid comprising a sequence having at least 70% identity to a protein coding sequence of SEQ ID NO: 7. For example, a functional ANXA2 protein may be encoded by a nucleic acid comprising a sequence having at least 75%, at least 80%, at least 85%, at least 90%, at least 95%, at least 96%, at least 97%, at least 98%, or at least 99% identity to a protein coding sequence of SEQ ID NO: 1.

In some embodiments, a PRRC2B protein comprises a sequence having at least 70% identity to the sequence of SEQ ID NO: 2 and maintains the functions described herein (e.g., capable of inducing cellular rejuvenation of a cell, for example, reducing levels of reactive oxygen species in a cell, and/or increasing cellular growth rate). For example, a functional PRRC2B protein may comprise a sequence having at least 75%, at least 80%, at least 85%, at least 90%, at least 95%, at least 96%, at least 97%, at least 98%, or at least 99% identity to the sequence of SEQ ID NO: 2.

In some embodiments, a MAP4 protein comprises a sequence having at least 70% identity to the sequence of SEQ ID NO: 3 and maintains the functions described herein (e.g., capable of inducing cellular rejuvenation of a cell, for example, reducing levels of reactive oxygen species in a cell, and/or increasing cellular growth rate). For example, a functional MAP4 protein may comprise a sequence having at least 75%, at least 80%, at least 85%, at least 90%, at least 95%, at least 96%, at least 97%, at least 98%, or at least 99% identity to the sequence of SEQ ID NO: 3.

In some embodiments, a BGN protein comprises a sequence having at least 70% identity to the sequence of SEQ ID NO: 4 and maintains the functions described herein (e.g., capable of inducing cellular rejuvenation of a cell, for example, reducing levels of reactive oxygen species in a cell, and/or increasing cellular growth rate). For example, a functional BGN protein may comprise a sequence having at least 75%, at least 80%, at least 85%, at least 90%, at least 95%, at least 96%, at least 97%, at least 98%, or at least 99% identity to the sequence of SEQ ID NO: 4.

In some embodiments, a CLTA protein comprises a sequence having at least 70% identity to the sequence of SEQ ID NO: 5 and maintains the functions described herein (e.g., capable of inducing cellular rejuvenation of a cell, for example, reducing levels of reactive oxygen species in a cell, and/or increasing cellular growth rate). For example, a functional CLTA protein may comprise a sequence having at least 75%, at least 80%, at least 85%, at least 90%, at least 95%, at least 96%, at least 97%, at least 98%, or at least 99% identity to the sequence of SEQ ID NO: 5.

In some embodiments, a RPS23 protein comprises a sequence having at least 70% identity to the sequence of SEQ ID NO: 6 and maintains the functions described herein (e.g., capable of inducing cellular rejuvenation of a cell, for example, reducing levels of reactive oxygen species in a cell, and/or increasing cellular growth rate). For example, a functional RPS23 protein may comprise a sequence having at least 75%, at least 80%, at least 85%, at least 90%, at least 95%, at least 96%, at least 97%, at least 98%, or at least 99% identity to the sequence of SEQ ID NO: 6.

### Nucleic Acids

The cells of the present disclosure, in some embodiments, comprise engineered nucleic acids. For example, an engineered nucleic acid may encode a protein having at least 70%, at least 75%, at least 80%, at least 85%, at least 90%, at least 95%, at least 96%, at least 97%, at least 98%, at least 99%, or 100% identity to a functional ANXA2 protein comprising the sequence of SEQ ID NO: 2. In some embodiments, an engineered nucleic acid may encode a protein having at least 70%, at least 75%, at least 80%, at least 85%, at least 90%, at least 95%, at least 96%, at least 97%, at least 98%, at least 99%, or 100% identity to a functional PRRC2B protein comprising the sequence of SEQ ID NO: 2. In some embodiments, an engineered nucleic acid may encode a protein having at least 70%, at least 75%, at least 80%, at least 85%, at least 90%, at least 95%, at least 96%, at least 97%, at least 98%, at least 99%, or 100% identity to a functional MAP4 protein comprising the sequence of SEQ ID NO: 3. In some embodiments, an engineered nucleic acid may encode a protein having at least 70%, at least 75%, at least 80%, at least 85%, at least 90%, at least 95%, at least 96%, at least 97%, at least 98%, at least 99%, or 100% identity to a functional BGN protein comprising the sequence of SEQ ID NO: 4. In some embodiments, an engineered nucleic acid may encode a protein having at least 70%, at least 75%, at least 80%, at least 85%, at least 90%, at least 95%, at least 96%, at least 97%, at least 98%, at least 99%, or 100% identity to a functional CLTA protein comprising the sequence of SEQ ID NO: 5. In some embodiments, an engineered nucleic acid may encode a protein having at least 70%, at least 75%, at least 80%, at least 85%, at least 90%, at least 95%, at least 96%, at least 97%, at least 98%, at least 99%, or 100% identity to a functional RPS23 protein comprising the sequence of SEQ ID NO: 6.

An engineered nucleic acid is a polynucleotide (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. The Gateway cloning method may also be used (ThermoFisher). Gateway cloning allows transfer of DNA sequences between plasmids using enzymatic recombination and a system of genetic selectable markers for specific assembly and growth of plasmids bearing engineered DNA sequences in E. coli.

In some embodiments, an engineered nucleic acid comprises a promoter operably linked to an open reading frame. A promoter is a nucleotide sequence to which RNA polymerase binds to initial transcription (e.g., ATG). Promoters are typically located directly upstream from (at the 5′ end of) a transcription initiation site. In some embodiments, a promoter is a heterologous promoter. A heterologous promoter is not naturally associated with the open reading frame to which is it operably linked.

In some embodiments, a promoter is an inducible promoter. An inducible promoter may be regulated in vivo by a chemical agent, temperature, or light, for example. Inducible promoters enable, for example, temporal and/or spatial control of gene expression. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells). In some embodiments, the inducible promoter is a tetracycline-inducible promoter. In some embodiments, the inducible promoter is a doxycycline-inducible promoter. In other embodiments, a promoter is a constitutive promoter (active in vivo, unregulated).

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

Vectors used for delivery of an engineered nucleic acids include viral vectors and non-viral vectors. Non-limiting examples of viral vectors include retrovirus, adenovirus, adeno-associated virus (AAV), and herpes simplex virus. Non-limiting examples of non-viral vectors include minicircles, plasmids, bacterial artificial chromosomes (BACs), and yeast artificial chromosomes. Transposon-based systems, such as the piggyBac™ system (e.g., Chen et al. Nature Communications. 2020; 11(1): 3446), may be used as a vector system to deliver an engineered nucleic acid. Other non-limiting examples include nanoparticle-based systems, such as lipid nanoparticles.

Transfection refers to the uptake of exogenous (e.g., engineered) nucleic acids (e.g., DNA or RNA) by a cell. A cell has been “transfected” when an exogenous nucleic acid has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13: 197. Such techniques can be used to introduce one or more engineered nucleic acid into cells. The term refers to both stable and transient uptake of the nucleic acid (e.g., DNA or RNA). For example, transfection can be used for transient uptake of mRNA encoding ANXA2, PRRC2B, MAP4, BGN, CLTA, and/or RPS23 protein into cells in need of rejuvenation.

### Cellular Rejuvenation and Regeneration

Some aspects provide methods of inducing cellular rejuvenation of a cell, for example, to counteract the effects of aging. Aging in mammals has been summarized and categorized into nine “hallmarks” of aging: genomic instability, telomere attrition, epigenetic alterations, loss of proteostasis, deregulated nutrient sensing, mitochondrial dysfunction, cellular senescence, stem cell exhaustion, and altered intracellular communication (López-Otin C et al. Cell 2013; 153: 1194-1217). Cellular rejuvenation is a process that not only delays aging but reverts it, leading to a phenotypically and functionally younger cell. Cellular rejuvenation can decrease or eliminate age-accumulated damage and aging hallmarks collected during the life of a cell. Thus, “inducing cellular rejuvenation” refers to a process (method) that initiates the reversal of a hallmark of aging. Induction of cellular rejuvenation may be assessed, for example, by assessing the transcriptomic profile, gene expression of one or more nuclear and/or epigenetic markers, proteolytic activity, mitochondria health and/or function, expression of one or more SASP cytokines, or the methylation landscape of the cell, compared to a control (e.g., a reference value, for example, obtained from a young cell or an aged cell). A “rejuvenated cell” is an aged cell that has been transiently or stably transfected with a protein or a nucleic acid encoding the protein such that the cell has a transcriptomic profile of a younger cell while still retaining one or more cell identity markers. Thus, a “rejuvenated cell” is an aged cell that has been transiently or stably transfected with an ANXA2 protein or a nucleic acid encoding an ANXA2 protein such that the cell has a transcriptomic profile of a younger cell while still retaining one or more cell identity markers. A “rejuvenated cell” may also be an aged cell that has been transiently or stably transfected with an PRRC2B, MAP4, BGN, CLTA, and/or RPS23 protein or a nucleic acid encoding an PRRC2B, MAP4, BGN, CLTA, and/or RPS23 protein such that the cell has a transcriptomic profile of a younger cell while still retaining one or more cell identity markers.

A transcriptomic profile refers to the set of all RNA molecules in one cell or a population of cells. It is sometimes used to refer to all RNAs, or just mRNA, depending on the particular experiment. It differs from the exome in that it includes only those RNA molecules found in a specified cell population, and usually includes the amount or concentration of each RNA molecule in addition to the molecular identities. Methods of obtaining a transcriptomic profile include DNA microarrays and next-generation sequencing technologies such as RNA-Seq. Transcription can also be studied at the level of individual cells by single-cell transcriptomics. There are two general methods of inferring transcriptome sequences. One approach maps sequence reads onto a reference genome, either of the organism itself (whose transcriptome is being studied) or of a closely related species. The other approach, de novo transcriptome assembly, uses software to infer transcripts directly from short sequence reads.

In some embodiments, a transcriptomic profile of a rejuvenated cell becomes more similar to a transcriptomic profile of a young cell. For example, the transcriptomic profile of a rejuvenated cell may comprise an increase or decrease in gene expression (e.g., toward the expected levels in young cells) of one or more genes selected from COL1A1, COL3A1, TIMP1, TIMP2, SMAD2, SMAD3, CTGF, TGFb1, TET2, TET3, SIRT1, SIRT6, HIF-1a, PTEN, PCK1, PPARG, CISD2.

In some embodiments, a rejuvenated cell exhibits increased gene expression of one or more nuclear and/or epigenetic markers compared to a control (e.g., a reference value). For example, the marker may be selected from HP 1 gamma, H3K9me3, lamina support protein LAP2alpha, and SIRT1 protein.

In some embodiments, a rejuvenated cell exhibits young-like proteostatic activity compared to a control. For example, proteostatis activity may be measured as increased cell autophagosome formation, increased chymotrypsin-like proteasome activity, or a combination thereof. In some embodiments, changes in age and inflammation-associated proteases such as MMPs can be measured proteomically or through biochemical detection of MMP enzymatic activity.

In some embodiments, a rejuvenated cell exhibits improved mitochondria health and/or function compared to a control. For example, the improved mitochondria health and function may be measured as increased mitochondria membrane potential, decreased reactive oxygen species (ROS), or a combination thereof.

In some embodiments, a rejuvenated cell exhibits decreased expression of one or more SASP cytokines compared to a control. For example, the SASP cytokines comprise one or more of IL18, IL1A, GROA, IL22, and IL9.

In some embodiments, a rejuvenated cell exhibits reversal of the methylation landscape. Reversal of the methylation landscape may be measured by Horvath clock estimation or other clocks focused on sites that lose and/or gain methylation over time.

In some embodiments, the control is a young cell or an aged cell or a reference value obtained from a young cell or an aged cell or a pool of such cells.

In some embodiments, induction of cellular rejuvenation leads to a reduction or inhibition of cellular senescence. For example, senescence-associated β-galactosidase activity of a cell may be decreased. As another example, proteasomal activity of a cell may be decreased.

Herein, an “effective amount” of a ANXA2 protein (or other protein such as PRRC2B, MAP4, BGN, CLTA, or RPS23) or a nucleic acid encoding the ANXA2 protein (or other protein such as PRRC2B, MAP4, BGN, CLTA, or RPS23) is an amount sufficient to initiate the reversal of a hallmark of aging, for example, a younger transcriptomic profile (more similar to a younger cell), increased gene expression of one or more nuclear and/or epigenetic markers, increased proteostasic activity, improved mitochondria health and/or function, decreased expression of one or more SASP cytokines, or reversal of the methylation landscape of the cell, compared to a control (e.g., a reference value, for example, obtained from a young cell or an aged cell). In some embodiments, mitochondrial health is measured by the signal intensity of mitochondria stained with MitoTracker Deep Red and measured by microscopy. An “effective amount” of a ANXA2 protein (or other protein such as PRRC2B, MAP4, BGN, CLTA, or RPS23) or a nucleic acid encoding the ANXA2 protein (or other protein such as PRRC2B, MAP4, BGN, CLTA, RPS23) also include an amount sufficient to decrease levels of reactive oxygen species in a cell or increase growth rate of a cell.

Cellular senescence, in some embodiments, is characterized by, and may be induced by, changes in chromatin organization that induce changes in gene expression, such as for example, the “senescence-associated secretory phenotype” (“SASP”) in which senescent cells secrete inflammatory cytokines and mitokines that can damage or alter the surrounding tissue. Thus, an SASP is an array of diverse cytokines, chemokines, growth factors, and proteases that are a characteristic feature of senescent cells. Senescent cells are stable, non-dividing cells that are still metabolically active and exhibit the upregulation of a wide range of genes including those that encode secreted proteins, such as inflammatory cytokines, chemokines, extracellular matrix remodeling factors, and growth factors. These secreted proteins function physiologically in the tissue microenvironment, in which they could propagate the stress response and communicate with neighboring cells. This SASP phenotype uncovers the paracrine function of senescent cells and is an important characteristic that distinguishes senescent cells from non-senescent, cell cycle-arrested cells, such as quiescent cells and terminally differentiated cells. SASP cytokines are cytokines produced specifically by senescent cells to create the senescence-associated secretory phenotype. These cytokines include but are not limited to IL18, IL1A, GROA, IL22, and IL9.

Studies have indicated that cellular senescence is associated with age-related conditions, including thinning of the epidermis, skin wrinkling, hair loss and greying hair, reduction in muscle thickness and muscle strength, increased incidence of inflammation, metabolic disturbances, loss of endurance, and age-associated diseases. In addition, cellular senescence is believed to contribute to difficulties associated with wound healing.

Accordingly, preventing cells from undergoing cellular senescence, or reversing cellular senescence in cells which have undergone cellular senescence, would be advantageous to treat various age-related conditions, would healing, and cosmetic applications.

There are several assays used by researchers for detecting senescence. In some embodiments, rejuvenation and senescence are detected by measuring organelle number in a cell. In some embodiments, rejuvenation and senescence are detected by measuring levels of reactive oxygen species in a cell. In some embodiments, rejuvenation and senescence are detected by measuring growth rate of a cell. The colorimetric substrate for β-gal, 5-bromo-4-chloro-3-indolyl-β-D-galactopyranoside, known as x-gal has long been used to detect metabolic activity in cells in vitro. On hydrolysis by β-gal, x-gal is converted into a blue precipitate that can be detected using microscopy. While the x-gal assay is viewed as the “gold standard” method, it is limited in that it is a colorimetric assay. C12FDG is a fluorescent alternative to x-gal. If also functions as a β-gal substrate but has the drawback of leaking out of the cell within a short period of time. In flow cytometry, a combination of antibody markers such as p16ARF and p21 may be used. An alternative is CellEvent™ Senescence Green Reagent. It offers a sensitive, fluorescent substrate for 3-gal that can be used for the detection of senescent cells in flow cytometry assays and imaging applications. It offers the advantage of not only being a fluorescent substrate for 3-gal but does not leak out of cells with time due to its ability to covalently bind to intracellular proteins.

### Senescence-Associated Beta-Galactosidase Activities

Some aspects provide a method for inducing cellular rejuvenation of a cell comprising contacting the cell with an effective amount of a ANXA2 protein (or other protein such as PRRC2B, MAP4, BGN, CLTA, or RPS23) or a nucleic acid encoding the ANXA2 protein (or other protein such as PRRC2B, MAP4, BGN, CLTA, RPS23), wherein the effective amount is sufficient to decrease senescence-associated beta-galactosidase (SA-βgal) activity. SA-βgal activity, detectable at pH 6.0, permits the identification of senescent cells in culture and mammalian tissues. SA-βgal activity may be assessed using, for example, a cytochemical protocol suitable for the histochemical detection of individual senescent cells both in culture and tissue biopsies. As another example, a method based on the alkalinization of lysosomes, followed by the use of 5-dodecanoylaminofluorescein di-β-D-galactopyranoside (C12FDG), a fluorogenic substrate for βgal activity, may be used. See, e.g., Debacq-Chainiaux, F et al. Nature Protocols 2009; 4: 1798-1806 for exemplary protocols. The cytochemical method is applicable to tissue sections and requires simple reagents and equipment. The fluorescence-based methods have the advantages of being more quantitative and sensitive.

In some embodiments, the effective amount is sufficient to decrease the SA-βgal activity by at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, or at least 100% relative to a control. In some embodiments, the effective amount is sufficient to decrease the SA-βgal activity of the cell by about 50% to about 100%.

### Proteasomal Activity

Some aspects provide a method for inducing cellular rejuvenation of a cell comprising contacting the cell with an effective amount of a ANXA2 protein (or other protein such as PRRC2B, MAP4, BGN, CLTA, RPS23) or a nucleic acid encoding the ANXA2 protein (or other protein such as PRRC2B, MAP4, BGN, CLTA, RPS23), wherein the effective amount is sufficient to decrease the proteasomal activity of the cell. Proteasome activity refers to the degradation of unneeded or damaged proteins by the proteasome, a protein complex, through proteolysis, a chemical reaction that breaks peptide bonds. Chymotrypsin-like proteasome activity is a distinct catalytic activity of the proteasome.

In some embodiments, the effective amount is sufficient to increase the proteasomal activity of the cell by at least 25%, at least 30%, at least 35%, at least 40%, at least 45%, or at least 50%, relative to a control. In some embodiments, the effective amount is sufficient to increase the proteasomal activity of the cells by about 50% to about 100%.

### Rejuvenated and Regenerated Cells and Tissue, and Methods of Production

Some aspects methods of inducing rejuvenation and/or regeneration of a cell (one or more cells) by overexpression of ANXA2 protein. The term “rejuvenation,” as used herein, may be used interchangeably with the term “regeneration.” Other aspects methods of inducing rejuvenation of a cell (one or more cells) by overexpression of a PRRC2B, MAP4, BGN, CLTA, or RPS23 protein. In some embodiments, functionality is restored in the cell. Functionalities commonly described in literature as aging endpoints include, for example, mitochondrial function, proteolytic activity, heterochromatin levels, histone methylation, nuclear lamina polypeptides, cytokine secretion, or senescence.

In some embodiments, rejuvenation of a cell or cells state, function, and predicted age is defined as the conversion of an aged cell to an omically-defined state which is quantitatively indistinguishable similar to from that of a younger cell. This state may be measured by, but is not limited to, transcriptomic, proteomic, epigenomic, methylomic, metabolomic, or microscopically-measured phenomic profiling or any multi-omic combination thereof.

In some embodiments quantitative determination of young and aged omic cell states can be performed through computational methods including but not limited to machine learning techniques which identify the most salient features from an omic data set differentiating a pair of differently-aged cells. A machine learning model so-trained can assess the rejuvenative potential ability of a given treatment by classifying the omic measure(s) of a cell or cells subjected to that treatment and assigning the profile to either aged or young states. Rejuvenation by this measure would then result in an otherwise aged cell or cells being classified as an omically-defined young cell as a consequence of a given treatment.

In some embodiments, the machine learning technique may comprise a supervised machine learning algorithm or supervised machine learning classifier configured to analyze a morphological signature and/or a functional signature of a cell in vitro. In some embodiments, an age of the cell in vitro that is over 55yo indicates that the cell is functionally aged. In some embodiments, an age of the cell in vitro that is under 25yo indicates that the cell is functionally young.

In some embodiments, the method for identifying the cell's state, function, and predicted age comprises contacting a cell with first binding reagent, phalloidin, followed by a second binding reagent, wheat germ agglutinin, followed by a third binding reagent, Hoechst 33342, followed by a fourth binding reagent, MitoTracker Deep Red, followed by a fifth binding reagent, concanavalin A, and followed by a sixth binding reagent, SYTO 14. In some embodiments, the first, second, third, fourth, fifth, and/or sixth markers are located on or in the cell's nucleus, extranuclear DNA, cytoplasmic actin, plasma membrane, endoplasmic reticulum, cytoplasm, cytoskeleton, Golgi apparatus/apparati, mitochondria, and/or RNA.

In some embodiments, the cell's morphological fingerprint comprises the cell's size and/or the cell's shape and/or other morphological properties such as eccentricity, form factor, and solidity. In some embodiments, the cell's functional fingerprint comprises one or more of a marker representative of a specific cell organelle and/or compartment.

In some embodiments, the cell samples may undergo additional processing in order to generate -omics data. This can involve bulk, single cell, in situ or in suspension sequencing of -omics measurements including but not limited to genomics, methylomics, transcriptomics, proteomics, and/or metabolomics. The -omics data may then get combined with the functional fingerprint to enable an improved cell's predicted age.

In some embodiments, methods define the plurality of a cell's state and function using machine learning methods that analyze, characterize, and index the cell's morphological and/or functional fingerprint which enable generation of a cell's age index. In some embodiments, the raw morphological and/or functional imaging data may be combined with multi-omics data (genomics, methylomics, transcriptomics, proteomics, and/or metabolomics) and/or other biomarker data thereby producing a dataset of features which enable generation of an improved cell's age index. In some embodiments, methods define the plurality of a tissue's state and function using machine learning methods that analyze, characterize, and index the tissue's morphological and/or functional fingerprint which enable generation of a tissue's age index.

One aspect of the present disclosure provides methods for measuring whether a perturbation affects a cell's transition from a cell's old index (reference fingerprint) to a cell's younger index (altered fingerprint). The differential between the reference and the altered cell age index defines a cell's “Youth score”. A “rejuvenated cell” has high Youth score. In some embodiments, the perturbation may be enabled via a nucleic acid, virus, protein, peptide, antibody, antigen, small molecule drug, a second different from the first cell type or a combination thereof.

In some embodiments, different machine learning methods may be needed for different cell and tissue types. The exact model structure may differ depending on the source of the sample, and the learned weights of the model may differ depending on the processes used during sample preparation and image data collection (factors include microscopy equipment and staining workflows). Over time, these models may become more generalizable across multiple sample types and preparation processes.

In some embodiments, an ANXA2, PRRC2B, MAP4, BGN, CLTA, and/or RPS23 protein or a nucleic acid (DNA or RNA, e.g., RNA) encoding ANXA2, PRRC2B, MAP4, BGN, CLTA, and/or RPS23 protein can be used to rejuvenate a variety of cell types, including fibroblasts, hematopoietic stem cells, endothelial cells, chondrocytes, skeletal muscle stem cells, keratinocytes, mesenchymal stem cells and corneal epithelial cells, optionally while retaining cell identity.

Some aspects provide methods of inducing rejuvenation of a cell that comprise transfecting a cell with of inducing rejuvenation of a cell, thereby producing inducing rejuvenation of a cell. In some embodiments, a rejuvenate cell is produced.

In some embodiments, a rejuvenated cell had a phenotype or activity profile similar to a young cell. The phenotype or activity profile includes one or more of the transcriptomic profile, gene expression of one or more nuclear and/or epigenetic markers, proteolytic activity, mitochondrial health and function, SASP cytokine expression, and methylation landscape.

In some embodiments, a rejuvenated cell has a transcriptomic profile that is more similar to the transcriptomic profile of young cells. In some embodiments, the transcriptomic profile of a rejuvenated cell includes an increase or a decrease in gene expression (e.g., toward the expected levels in young cells) of one or more genes selected from COL1A1, COL3A1, TIMP1, TIMP2, SMAD2, SMAD3, CTGF, TGFb1, TET2, TET3, SIRT1, SIRT6, HIF-1a, PTEN, PCK1, PPARG, CISD2.

In some embodiments, a rejuvenated cell exhibits increased gene expression of one or more nuclear and/or epigenetic markers compared to a control (e.g., a reference value). In some embodiments, the one or more nuclear and/or epigenetic markers is selected from HP 1 gamma, H3K9me3, lamina support protein LAP2alpha, and SIRT1 protein. In some embodiments, the rejuvenated cell exhibits increased gene expression of HP 1 gamma. In some embodiments, the rejuvenated cell exhibits increased gene expression of H3K9me3. In some embodiments, the rejuvenated cell exhibits increased gene expression of lamina support protein LAP2alpha. In some embodiments, the rejuvenated cell exhibits increased gene expression of SIRT1 protein. In some embodiments, the rejuvenated cell exhibits increased gene expression of HP 1 gamma, H3K9me3, lamina support protein LAP2alpha, and SIRT1 protein.

In some embodiments, a rejuvenated cell has a proteolytic activity that is more similar to the proteolytic activity of young cells. In some embodiments, the proteolytic activity is measured as increased cell autophagosome formation, increased chymotrypsin-like proteasome activity, or a combination thereof. In some embodiments, the proteolytic activity is measured as increased cell autophagosome formation. An autophagosome is a spherical structure with double layer membranes. It is a key structure in macroautophagy, the intracellular degradation system for cytoplasmic contents (e.g., abnormal intracellular proteins, excess or damaged organelles) and also for invading microorganisms. After formation, autophagosomes deliver cytoplasmic components to the lysosomes. The outer membrane of an autophagosome fuses with a lysosome to form an autolysosome. The lysosome's hydrolases degrade the autophagosome-delivered contents and its inner membrane. In some embodiments, the proteolytic activity is measured as increased chymotrypsin-like proteasome activity. In some embodiments, the proteolytic activity is measured as increased cell autophagosome formation and increased chymotrypsin-like proteasome activity.

In some embodiments, the rejuvenated cell exhibits improved mitochondria health and function compared to a control (e.g., a reference value). In some embodiments, improved mitochondria health and function is measured as increased mitochondria membrane potential, decreased reactive oxygen species (ROS), or a combination thereof. In some embodiments, improved mitochondria health and function is measured as increased mitochondria membrane potential. In some embodiments, improved mitochondria health and function is measured as decreased reactive oxygen species (ROS). In some embodiments, improved mitochondria health and function is measured as increased mitochondria membrane potential and decreased reactive oxygen species (ROS).

In some embodiments, a rejuvenated cell exhibits decreased expression of one or more SASP cytokines compared to a control (e.g., a reference value). In some embodiments, the one or more SASP cytokines include IL18, IL1A, GROA, IL22, and IL9. In some embodiments, the rejuvenated cell exhibits decreased expression of IL18. In some embodiments, the rejuvenated cell exhibits decreased expression of IL1A. In some embodiments, the rejuvenated cell exhibits decreased expression of GROA. In some embodiments, the rejuvenated cell exhibits decreased expression of IL22. In some embodiments, the rejuvenated cell exhibits decreased expression of IL9. In some embodiments, the rejuvenated cell exhibits decreased expression of IL18, IL1A, GROA, IL22, and IL9.

In some embodiments, a rejuvenated cell exhibits reversal of the methylation landscape. In some embodiments, the reversal of the methylation landscape is measured by Horvath clock estimation.

In some embodiments, a reference value is obtained from a young cell or an aged cell.

It should be understood that any “increase” or “decrease” (e.g., reduce or reduction) of a characteristic and/or function exhibited by a cell is relative to or compared to a control, such as a reference value (e.g., obtained from a young cell or an aged cell).

Cellular senescence is the disruption of cell proliferation and function. During cellular senescence, there is a loss of the ability of the cell to proliferate, although the cell continues to remain viable and metabolically active.

Many cell types undergo cellular senescence following a large number of cycles of cell division. This barrier to further proliferation following many cycles of cell division has been termed replicative senescence. Replicative senescence is thought to be due to shortening of the cell's telomeres with each successive cell division, causing cells to reach a point (their so-called “Hayflick limit”) at which a DNA damage response is triggered, leading ultimately to induction of proliferation arrest and cellular senescence. Cellular senescence can also be induced in the absence of telomere loss or dysfunction. DNA damage may take the form of chromosomal dysfunction such as aneuploidy arising from unequal chromosome segregation during mitosis, DNA strand breaks, or chemical modification of DNA. Cellular senescence may also be induced by a DNA damage response (DDR) which may or may not reflect actual DNA damage.

### Cell Transfection Methods

In embodiments, transfecting cells with an ANXA2, PRRC2B, MAP4, BGN, CLTA, and/or RPS23 protein or a nucleic acid encoding an ANXA2, PRRC2B, MAP4, BGN, CLTA, and/or RPS23 protein may be accomplished by a transfection method selected from electroporation (e.g., nucleofection, lipofectamine and LT-1 mediated transfection, dextran-mediated transfection, calcium phosphate precipitation, polybrene mediated transfection, encapsulation of the nucleic acid (e.g., mRNA) in liposomes, and direct microinjection. In some embodiments, transfecting cells with protein or nucleic acid may be accomplished by lipofectamine and LT-1 mediated transfection. In some embodiments, transfecting cells with protein or nucleic acid may be accomplished by dextran-mediated transfection. In some embodiments, transfecting cells with protein or nucleic acid may be accomplished by calcium phosphate precipitation. In some embodiments, transfecting cells with protein or nucleic acid may be accomplished by polybrene mediated transfection. In some embodiments, transfecting cells with protein or nucleic acid may be accomplished by electroporation. In some embodiments, transfecting cells with protein or nucleic acid may be accomplished by encapsulation of the mRNAs in liposomes. In some embodiments, transfecting cells with protein or nucleic acid may be accomplished by direct microinjection.

### Cell Types

The methods, in some aspects, are used to induce cellular rejuvenation in cells. In some embodiments, the methods provided herein may be applied to any type of cell in need of rejuvenation. Cells may be intact live cells, naturally occurring or modified. A cell may be isolated from other cells, mixed with other cells in a culture, or within a tissue (partial or intact) or an organism. The methods described herein can be performed, for example, on a sample comprising a single cell, a population of cells, or a tissue or organ comprising cells. The methods can also be used to deliver nucleic acids or proteins to cells in vivo. The cells chosen for rejuvenation, in some embodiments, depends on the desired therapeutic effect for treating an age-related disease or condition.

The rejuvenative ability of the delivered nucleic acids or proteins affects broad cellular processes required to drive whole phenotypic omic rejuvenation, can be expected to be robust to differences in cell-type specific physiology. Just as robust cell state reprogramming driven by Yamanaka factors is effective in a variety [all] of functionally, physiologically, and developmentally distinct cell types, our rejuvenation nucleic acids or proteins may have general efficacy in non-fibroblast, non-mesodermal cell types.

In some embodiments, the cell is an adult stem cell. In some embodiments, the adult stem cell is selected from a hematopoietic stem cell, epithelial stem cell, neuronal stem cell and mesenchymal stem cell. In some embodiments, the mesenchymal stem cell is selected from a fibroblast, myocyte, adipocyte, chondrocyte, and osteocyte. In some embodiments, the hematopoietic stem cell is a T or NK cell. In some embodiments, the T cell is a CD4⁺CD8⁺ cell, CD4⁺ cell (Th1, Th2, Th17, or Treg), a naive T cell, a central memory T cell, or an effector memory T cell.

In some embodiments, the cell is selected from an ectoderm, endoderm, mesoderm and germ cell. In some embodiments, the ectoderm cell is keratinocyte, pigment cell or neuronal cell. In some embodiments, the endoderm cell is a liver cell, lung cell, pancreatic cell or thyroid cell. In some embodiments, the mesoderm cell is a cardiac muscle cell, skeletal muscle cell, smooth muscle cell, kidney tubule cell or a red blood cell. In some embodiments, the germ cell is an egg or sperm cell.

In some embodiments, a cell is a mammalian cell (e.g., cell derived from a mammalian subject suitable for transplantation into the same or a different subject). In some embodiments, a cell is a human cell. In some embodiments, a cell is from an elderly subject.

A cell may be xenogeneic, autologous, or allogeneic. A cell can be a primary cell obtained directly from a mammalian subject. The cell may also be a cell derived from the culture and expansion of a cell obtained from a subject. In some embodiments, the cell has been genetically engineered to express an ANXA2, PRRC2B, MAP4, BGN, CLTA, and/or RPS23 protein and/or a nucleic acid encoding an ANXA2, PRRC2B, MAP4, BGN, CLTA, and/or RPS23 protein.

The methods, in some aspects, comprising contacting (e.g., transfecting) a cell with a therapeutically effective amount of an ANXA2, PRRC2B, MAP4, BGN, CLTA, and/or RPS23 protein or a nucleic acid encoding the ANXA2, PRRC2B, MAP4, BGN, CLTA, and/or RPS23 protein. In some embodiments, a cell is selected from fibroblasts, hematopoietic stem cells, endothelial cells, chondrocytes, skeletal muscle stem cells, keratinocytes, mesenchymal stem cells and corneal epithelial cells. In some embodiments, a cell is a fibroblast. In some embodiments, a cell is a hematopoietic stem cell. In some embodiments, a cell is an endothelial cell. In some embodiments, a cell is a chondrocyte. In some embodiments, a cell is a skeletal muscle stem cell. In some embodiments, a cell is a keratinocyte. In some embodiments, a cell is a mesenchymal stem cell. In some embodiments, a cell is a corneal epithelial cell.

A fibroblast is a type of cell that contributes to the formation of connective tissue, a fibrous cellular material that supports and connects other tissues or organs in the body. Fibroblasts secrete collagen proteins that help maintain the structural framework of tissues. Dermal fibroblasts are the main cell type present in skin connective tissue (dermis). Fibroblasts interact with epidermal cells during hair development and in interfollicular skin. Moreover, they play an essential role during cutaneous wound healing and in bioengineering of skin. Detailed procedures for establishing and maintaining primary cultures of adult human dermal fibroblasts are known (see, e.g., Kisiel et al. Methods Mol Biol. 2019; 1993:71-78).

In some embodiments, a rejuvenated fibroblast exhibits a transcriptomic profile similar to a transcriptomic profile of young fibroblasts. In some embodiments, a rejuvenated fibroblast exhibits an increased gene expression of one or more nuclear and/or epigenetic markers compared to a control (e.g., a reference value) as described above. In some embodiments, the rejuvenated fibroblasts have a proteolytic activity that is more similar to the proteolytic activity of young cells as described above. In some embodiments, a rejuvenated fibroblast exhibits improved mitochondria health and function compared to a control (e.g., a reference value) as described above. In some embodiments, a rejuvenated fibroblast exhibits a reversal of the methylation landscape.

In some embodiments, a rejuvenated endothelial cell exhibits a transcriptomic profile similar to a transcriptomic profile of young endothelial cells. In some embodiments, a rejuvenated endothelial cell exhibits increased gene expression of one or more nuclear and/or epigenetic markers compared to a control (e.g., a reference value) as described above. In some embodiments, rejuvenated endothelial cells have a proteolytic activity that is more similar to the proteolytic activity of young cells as described above. In some embodiments, a rejuvenated endothelial cell exhibits improved mitochondria health and function compared to a control (e.g., a reference value) as described above. In some embodiments, a rejuvenated endothelial cell exhibits a reversal of the methylation landscape.

In some embodiments, a rejuvenated chondrocyte exhibits reduced expression of inflammatory factors and/or and increased ATP and collagen metabolism. In some embodiments, the inflammatory factors include RANKL, iNOS2, IL6, IFNa, MCP3 and MIP1A. In some embodiments, a rejuvenated chondrocyte exhibits reduced expression of RANKL. In some embodiments, a rejuvenated chondrocyte exhibits reduced expression of iNOS2. In some embodiments, a rejuvenated chondrocyte exhibits reduced expression of IL6. In some embodiments, a rejuvenated chondrocyte exhibits reduced expression of IFNa. In some embodiments, a rejuvenated chondrocyte exhibits reduced expression of MCP3. In some embodiments, a rejuvenated chondrocyte exhibits reduced expression of MIP1A. In some embodiments, a rejuvenated chondrocyte exhibits reduced expression of RANKL, iNOS2, IL6, IFNa, MCP3 and MIP1A. In some embodiments, a rejuvenated chondrocyte exhibits increased ATP and collagen metabolism. In some embodiments, ATP and collagen metabolism is measured by one or more of increased ATP levels, decreased ROS and increased SOD2 expression, increased COL2A1 expression and overall proliferation by the chondrocyte. In some embodiments, ATP and collagen metabolism is measured by increased ATP levels. In some embodiments, ATP and collagen metabolism is measured by decreased ROS and increased SOD2 expression. In some embodiments, ATP and collagen metabolism is measured by increased COL2A1 expression and overall proliferation by the chondrocyte.

In some embodiments, a rejuvenated skeletal muscle stem cell exhibits higher proliferative capacity, enhanced ability to differentiate into myoblasts and muscle fibers, restored lower kinetics of activation from quiescence, ability to rejuvenate the muscular microniche, restore youthful force in the muscle, or a combination thereof.

In some embodiments, a rejuvenated keratinocytes exhibit higher proliferative capacity, reduced inflammatory phenotype, lower RNAKL and INOS2 expression, reduced expression of cytokines MIP1 A, IL6, IFNa, MCP3, increased ATP, increased levels of SOD2 and COL2A1 expression.

In some embodiments, a rejuvenated mesenchymal stem cell exhibits reduction in senescence parameters, increased cell proliferation, and/or a decrease in ROS levels. In some embodiments, a rejuvenated mesenchymal stem cell exhibits reduction in senescence parameters. In some embodiments, the senescence parameters include p16 expression, p21 expression and positive SA Gal staining. In some embodiments, a rejuvenated mesenchymal stem cell exhibits increased cell proliferation. In some embodiments, a rejuvenated mesenchymal stem cell exhibits a decrease in ROS levels. In some embodiments, a rejuvenated mesenchymal stem cell exhibits reduction in senescence parameters, increased cell proliferation, and a decrease in ROS levels.

In some embodiments, a rejuvenated corneal epithelial cell exhibits a reduction in senescence parameters. In some embodiments, the senescence parameters include one or more of expression of p21, expression of p16, mitochondria biogenesis PGC1a, and expression of inflammatory factor IL8. In some embodiments, the senescence parameters include p21. In some embodiments, the senescence parameters include expression of p16. In some embodiments, the senescence parameters include mitochondria biogenesis PGC1a. In some embodiments, the senescence parameters include expression of inflammatory factor IL8. In some embodiments, the senescence parameters include one expression of p21, expression of p16, mitochondria biogenesis PGC1a, and expression of inflammatory factor IL8.

In some embodiments, the cell is a stem cell. In some embodiments, the cell is a hematopoietic stem cell. A stem cell is a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells (iPSCs) are a type of pluripotent stem cell derived from adult cells that have been reprogrammed into an embryonic-like pluripotent state. Induced pluripotent stem cells can be derived, for example, from adult somatic cells such as skin or blood cells.

### Therapies

Aspects of the present disclosure relate to methods of the invention can be applied to treat one or more skin regions with one or more procedures to improve skin appearance and to rejuvenate skin at a molecular, cellular, and tissue levels. The methods described herein may also be used to trigger biological responses that may contribute to improved skin cell proliferation, skin cell motility, and/or tissue remodeling. Accordingly, compositions can be delivered to be useful for treatment of regions of the body with different sizes and geometries to include treatment of facial and other locations on the body. In other embodiments, the composition can be delivered in linear or curved shape, with varying size, geometry, and/or depth.

In some embodiments, nucleic acids presented in this disclosure relate to a method of are delivered in viral formulations to subject at an effective therapeutic amount to induce soft tissue enhancement and augmentation. In some embodiments, the composition is re-dosed into the soft tissue of the subject.

In some embodiments, nucleic acids, proteins and/or formulations thereof may be used as a treatment to a subject to induce “tissue rejuvenation” which manifests in enhanced production of dermal collagens and cellularity. On a macroscopic level, such treatments will lead to at least one of: the improvement of skin quality and appearance; the reduction of superficial depressions in the skin such as lines, wrinkles, and/or folds; and/or the enhancement of skin texture, and/or smoothness. The described herein treatments provide a novel therapeutic strategy for skin tissue rejuvenation.

In some embodiments, nucleic acids, proteins and/or compositions thereof may be used in a method to induce “tissue regeneration” which manifests in repair and/or augmentation of the soft tissue of a subject. In some embodiments, tissue regeneration refers to the restoration of tissue structure and/or function. In some embodiments, repair and/or augmentation of the soft tissue refers to restoration of more “youthful” appearance of facial skin as compared to an untreated “aged” skin. Aged skin is defined as skin with defects resulting from chronological aging and/or photoaging. In some embodiments, nucleic acids, proteins, viruses and/or compositions thereof are useful in medical aesthetics applications, such as to restore fine lines, wrinkles, folds, or scars.

Aspects of the present disclosure relate to a method of skin treatment relating to enhancing skin quality, condition, and/or appearance in a subject comprising administering to the subject any of the nucleic acids, proteins and/or compositions thereof. In some embodiments, the skin condition is selected from photoaging, intrinsic aging, aging induced by repeated facial expressions, loss of tone and gravity manifested in rough texture, sagging, wrinkles, furrows, folds, and any combinations thereof. In some embodiments, the treatment of the skin condition may be assessed using any appropriate method or scale known in the art, including quantitative improvement in wrinkles by the Lemperle Wrinkle Severity Scale, Global Aesthetic Improvement Scale, Optical Coherence Tomography, and qualitative changes in dermal and sub dermal collagen and cellularity. In some embodiments, new and/or custom scales can be used. Such scales provide better classification of fine lines as they use custom facial grading scales designed for specific areas on the face such as Forehead Lines Grading Scale and Crow's Feet Grading Scale. These scales were developed using computer-simulated images and are typically five-point scales that also include a midpoint.

Additionally, these scales include static and dynamic images, an important feature as we will likely primarily focus on static lines. These scales have been reported to have improved intra-rater variability.

In some embodiments, the rejuvenated HDF cells exhibit improved replicative lifespan and functionality. The rejuvenated HDF cells of the present disclosure can enhance the efficiency of all modalities of regenerative medicine by generating HDF cells that have enhanced capacity for expansion, improved motility, and augmented ability to engraft and enable improved wound healing in healthy and diseased skin.

In some embodiments, the rejuvenated cells described herein can be used to produce artificial tissue or organs ex vivo such as for organ transplantation purposes. Techniques for artificial tissue or organs production are known in the art. In some embodiments, the rejuvenated cells can be used to produce organ function enhancement directly administered in a subject. In some embodiments, the nucleic acids or proteins can be administered in vivo to enable organ function enhancement in a subject with a failing organ or organ transplant.

### Subjects

A subject, in some embodiments, is a human subject. In some embodiments, a subject is a young adult. A young adult subject is between the ages of 18 and 44 years old (including 18 and 44 years old).

In some embodiments, a subject is a middle-aged subject. A middle-aged subject may be between the ages of 45 and 65 years old (including 45 and 65 years old). In some embodiments, a middle-aged subject is between the ages of 50 and 65 years old or between the ages of 55 and 65 years old.

In some embodiments, a subject is an elderly subject. An elderly subject may be older than 65 years old. In some embodiments, an elderly subject is between the ages of 70 and 85 years old or between the ages of 75 and 85 years old.

In some embodiments, a subject is at least 50 years old. In some embodiments, a subject is at least 55 years old. In some embodiments, a subject is at least 60 years old. In some embodiments, a subject is at least 65 years old. In some embodiments, a subject is at least 70 years old. In some embodiments, a subject is at least 75 years old.

### Formulations

Aspects of the present disclosure provide a pharmaceutical composition for treating a disease or disorder associated with aging comprising a therapeutic efficacy leading changing a cell's state, function, an/and predicted age. The term therapeutic efficacy means a desired therapeutic effect such as decreasing predicted age and/or reversing an aging cellular and/or tissue level phenotypes.

The proteins and nucleic acids described herein may be formulated for a particular route of administration, which may depend, for example, on an intended therapy. In some embodiments, an ANXA2, PRRC2B, MAP4, BGN, CLTA, and/or RPS23 protein or a nucleic acid encoding the protein may be formulated for topical delivery. In some embodiments, an ANXA2, PRRC2B, MAP4, BGN, CLTA, and/or RPS23 protein or a nucleic acid encoding the protein is formulated for subcutaneous delivery. In some embodiments, an ANXA2, PRRC2B, MAP4, BGN, CLTA, and/or RPS23 protein or a nucleic acid encoding the protein is formulated for intravenous delivery. In some embodiments, an ANXA2, PRRC2B, MAP4, BGN, CLTA, and/or RPS23 protein or a nucleic acid encoding the protein is formulated for intramuscular delivery. The formulation, in some embodiments, includes an mRNA encoding an ANXA2, PRRC2B, MAP4, BGN, CLTA, and/or RPS23 protein and a lipid nanoparticle (LNP) or other lipid-based delivery system. An ANXA2, PRRC2B, MAP4, BGN, CLTA, and/or RPS23 protein or a nucleic acid encoding the protein, in other embodiments, is formulated for delivery via electroporation.

A protein or nucleic acid may be formulated, in some embodiments, with a pharmaceutically acceptable excipient, which an excipient that causes no significant adverse toxicological effects to a subject, such as a human subject.

### Routes of Administration

The route of administration of the proteins and nucleic acids described herein may vary depending on how they are formulated. Non-limiting examples of routes of administration include, topical, oral, nasal, intravenous, intramuscular, subcutaneous, and intraperitoneal, intraepidermal, transepidermal, intradermal, transdermal, or topical administration.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

### ADDITIONAL ASPECTS AND EMBODIMENTS

Additional aspects and embodiments are provided in the following numbered paragraphs:

1. A method, comprising:
contacting a cell with an effective amount of a protein or a nucleic acid encoding the protein, wherein the protein is:
ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IP05, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof;
   wherein the effective amount is sufficient to induce cellular rejuvenation and/or regeneration of the cell.
   2. A method, comprising:
administering to a subject an effective amount of a protein or a nucleic acid encoding the protein, wherein the protein is:
ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof;
   wherein the effective amount is sufficient to induce cellular rejuvenation of a cell in the subject.
   3. The method of paragraph 1 or paragraph 2, wherein the protein is selected from the group consisting of: ANXA2, BGN, CLTA, PRRC2B, MAP4, RPS23, AKT1, VCP, FAM129B, LMAN1, GUK1, SEC61A1, ELL2, RPN1, KIF5B, MSN, and TGOLN2.
   4. The method of paragraph 3, wherein the protein is selected from the group consisting of: ANXA2, BGN, CLTA, PRRC2B, MAP4, and RPS23.
   5. The method of paragraph 4, wherein the protein is ANXA2.
   6. The method of paragraph 4, wherein the protein is BGN.
   7. The method of paragraph 4, wherein the protein is CLTA.
   8. The method of paragraph 4, wherein the protein is PRRC2B.
   9. The method of paragraph 4, wherein the protein is MAP4.
   10. The method of paragraph 4, wherein the protein is RPS23.
   11. The method of any one of the preceding paragraphs, wherein the effective amount is sufficient to reduce reactive oxygen species (ROS) abundance in the cell, compared to an untreated cell.
   12. The method of paragraph 11, wherein the ROS abundance in the cell is reduced by at least 1%, at least 2%, at least 3%, at least 4%, at least 5%, at least 10%, at least 15%, at least 20%, or at least 25%, compared to an untreated cell.
   13. The method of any one of the preceding paragraphs, wherein the effective amount is sufficient to increase the growth rate of the cell, compared to an untreated cell.
   14. The method of paragraph 13, wherein the growth rate of the cell is increased by at least 1%, at least 2%, at least 3%, at least 4%, at least 5%, at least 10%, at least 15%, at least 20%, at least 25%, at least 30%, at least 35%, or at least 40%, compared to an untreated cell.
   15. The method of any one of the preceding paragraphs, wherein two or more proteins are selected, wherein the two or more proteins are encoded by two or more nucleic acids and the two or more nucleic acids are separated by a polycistronic element.
   16. The method of paragraph 15, wherein the polycistronic element is an IRES or a 2A sequence.
   17. The method of any one of the preceding paragraphs, wherein the effective amount is sufficient to induce an average cellular rejuvenation of at least 5 years, at least 10 years, at least 15 years, at least 20 years, at least 25 years, at least 30 years, at least 35 years, at least 40 years, at least 45 years, or at least 50 years.
   18. The method of paragraph 17, wherein average cellular rejuvenation is defined as the product of (A) the fraction of treated, misclassified cells in a supervised two-class machine learning ensemble, wherein an untreated training population of each class exceeds 1,000 cells; and (B) the age difference between the two classes.
   19. The method of any one of the preceding paragraphs, wherein the effective amount is sufficient to increase the signal intensity of cytoplasmic actin stained with phalloidin and measured by microscopy, the signal intensity of the Golgi apparatus/apparati stained with wheat germ agglutinin and measured by microscopy, the signal intensity of the plasma membrane stained with wheat germ agglutinin and measured by microscopy, the signal intensity of extranuclear DNA stained with Hoechst 33342 and measured by microscopy, the signal intensity of mitochondria stained with MitoTracker Deep Red and measured by microscopy, the signal intensity of the endoplasmic reticulum stained with concanavalin A and measured by microscopy, or the peripheral cellular signal intensity of RNA stained with SYTO 14 and measured by microscopy.
   20. The method of any one of the preceding paragraphs, wherein the effective amount is sufficient to decrease cytoplasmic volume as measured by microscopy, decrease cell volume as measured by microscopy, decrease cell surface area as measured by microscopy, or decrease the signal intensity of nuclear DNA stained with Hoechst 33342 and measured by microscopy.
   21. The method of any one of the preceding paragraphs, wherein the effective amount is sufficient to increase replicative life span of the cell by at least 25%, at least 30%, at least 35%, at least 40%, at least 45%, or at least 50%.
   22. The method of any one of the preceding paragraphs, wherein the effective amount is sufficient to increase total gene expression or extracellular matrix gene expression or collagen expression levels of the cell by at least 25%, at least 30%, at least 35%, at least 40%, at least 45%, or at least 50%.
   23. The method of any one of the preceding paragraphs, wherein the effective amount is sufficient to induce an average cellular rejuvenation of at least 20 years.
   24. The method of any one of the preceding paragraphs, wherein the cell is an adult stem cell.
   25. The method of paragraph 24, wherein the adult stem cell is hematopoietic stem cell, epithelial stem cell, neuronal stem cell or mesenchymal stem cell.
   26. The method of paragraph 25, wherein mesenchymal stem cell is fibroblast, myocyte, adipocyte, chondrocyte, or osteocyte.
   27. The method of paragraph 24, wherein the hematopoietic stem cell is a T cell or NK cell.
   28. The method of paragraph 27, wherein the T cell is a CD4⁺CD8⁺ cell, CD4⁺ cell (Th1, Th2, Th17, or Treg), a naive T cell, a central memory T cell, or an effector memory T cell.
   29. The method of any one of the preceding paragraphs, wherein the cell is an ectoderm, endoderm, mesoderm or germ cell.
   30. The method of paragraph 29, wherein the ectoderm cell is keratinocyte, pigment cell or neuronal cell.
   31. The method of paragraph 29, wherein the endoderm cell is a liver cell, lung cell, pancreatic cell or thyroid cell.
   32. The method of paragraph 29, wherein the mesoderm cell is a cardiac muscle cell, skeletal muscle cell, smooth muscle cell, kidney tubule cell or a red blood cell.
   33. The method of paragraph 29, wherein the germ cell is an egg or sperm cell.
   34. The method of any one of the preceding paragraphs, wherein the cell is selected from the group consisting of: fibroblasts, hematopoietic stem cells, endothelial cells, chondrocytes, skeletal muscle stem cells, keratinocytes, mesenchymal stem cells. and corneal epithelial cells.
   35. The method of paragraph 34, wherein the cells are fibroblasts.
   36. The method of paragraph 35, wherein the fibroblasts are human dermal fibroblasts.
   37. The method of any one of the preceding paragraphs, wherein the protein is a human, canine, feline, bovine, ovine, caprine, equine, murine, porcine or pachyderm protein.
   38. The method of paragraph 2, wherein the protein is delivered to skin tissue layers and structures including stratum corneum, epidermis, basement membrane, dermis, hair follicles, blood vessels, and sebaceous glands or and eccrine glands.
   39. The method of any one of the preceding paragraphs, wherein the nucleic acid comprises a heterologous promoter operably linked to an open reading frame.
   40. The method of any one of the preceding paragraphs, wherein the heterologous promoter is a constitutive promoter or an inducible promoter.
   41. The method of any one of the preceding paragraphs, wherein the regulatory sequence comprises a cell-specific promoter or a tissue-specific promoter.
   42. The method of any one of the preceding paragraphs, wherein the regulatory sequence comprises a promoter selected from the group consisting of: an hEfla promoter, an shEfla promoter (or truncated hEfla promoter), a CAG promoter (such as cytomegalovirus, chicken beta-actin intron, splice acceptor of the rabbit beta-globin gene), a CMV promoter, an hAAT promoter, a thyroid hormone-binding globulin promoter, an albumin promoter, a thyroxin-binding globulin (TBG) promoter, a hepatic control region (HCR)-ApoCII hybrid promoter, a CASI promoter, an HCR-hAAT hybrid promoter, an hAAT promoter combined with mouse albumin gene enhancer (Ealb) element, and an apolipoprotein E promoter.
   43. The method of any one of the preceding paragraphs, wherein the nucleic acid is operably linked to a 3′ untranslated region for RNA stability and expression in mammalian cells.
   44. The method of paragraph 43, wherein the 3′ untranslated region comprises a WPRE sequence, a WPRE3 sequence, an SV40 late polyadenylation signal (e.g., truncated), an HBG polyadenylation signal, a rabbit beta-globin polyadenylation signal, a bovine bgpA, an ETC polyadenylation signal, or any combination thereof.
   45. The method of any one of the preceding paragraphs, wherein the protein is delivered in a viral vector.
   46. The method of paragraph 45, wherein the viral vector is an AAV vector.
   47. The method of paragraph 46, wherein the AAV vector is derived from an AAV serotype selected from the group consisting of: AAV1, AAV2, AAV3, AAV4, AAV5, AAV6, AAV6.2, AAV7, AAV8, AAV9, AAV10, AAV11, AAV12, AAV13, AAVrh8, AAVrh10, and AAVrh32.
   48. The method of paragraph 47, wherein the AAV vector is derived from an AAV2 serotype.
   49. The method of paragraph 47, wherein the AAV vector is derived from an AAV6 serotype.
   50. The method of any one of the preceding paragraphs, further comprising contacting the cell with the protein or administering the protein directly to the subject.
   51. The method of any one of the preceding paragraphs, further comprising contacting the cell with the protein or administering to the subject the nucleic acid comprising an open reading frame encoding the protein.
   52. The method of any one of the preceding paragraphs, wherein the nucleic acid is delivered in a non-viral vector or a viral vector.
   53. The method of any one of the preceding paragraphs, wherein the contacting comprises transfecting the cell.
   54. The method of any one of the preceding paragraphs, wherein the nucleic acid comprises DNA, RNA, or a combination thereof.
   55. The method of any one of the preceding paragraphs, wherein the protein comprises a sequence that is at least 90% identical to any one of SEQ ID NOs: 1-6.
   56. The method of any one of the preceding paragraphs, wherein the nucleic acid comprises an amino acid sequence that is at least 90% identical to any one of SEQ ID NOs: 7-12.
   57. A method, comprising:
overexpressing in a cell a nucleic acid encoding a protein, wherein the protein is:
ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IP05, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof;
   wherein the overexpressing produces an effective amount of the protein in the cell, and wherein the effective amount is sufficient to induce cellular rejuvenation in the cell.
   58. The method of paragraph 57, wherein the protein is selected from ANXA2, BGN, CLTA, PRRC2B, MAP4, RPS23, AKT1, VCP, FAM129B, LMAN1, GUK1, SEC61A1, ELL2, RPN1, KIF5B, MSN, and TGOLN2.
   59. The method of paragraph 58, wherein the protein is an endogenous protein and the method comprises activating expression or activity of the endogenous protein at a level that is higher than a baseline level.
   60. The method of any one of the preceding paragraphs, wherein the subject has been diagnosed or is at risk of being diagnosed with a skin condition.
   61. The method of paragraph 60, wherein the skin condition is photoaging, intrinsic aging, aging induced by repeated facial expressions, loss of tone and gravity manifested in rough texture, sagging, wrinkles, furrows, folds, or any combinations thereof.
   62. A cell, comprising:
an engineered nucleic acid encoding a protein, wherein the protein is:
ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof.
   63. The cell of paragraph 62, wherein the protein is selected from the group consisting of: ANXA2, BGN, CLTA, PRRC2B, MAP4, RPS23, AKT1, VCP, FAM129B, LMAN1, GUK1, SEC61A1, ELL2, RPN1, KIF5B, MSN, and TGOLN2.
   64. The cell of paragraph 62 or paragraph 63, wherein the protein is a recombinant purified peptide sequence.
   65. The cell of paragraph 64, wherein the purified peptide is introduced to cells through non-covalent complexation with protein, lipid, synthetic polymeric, cell-binding ligand agents, or pore-forming agents.
   66. The cell of paragraph 64, wherein the purified peptide is introduced to cells through chemical formulations which induce entry into cells through osmotic shock.
   67. The cell of paragraph 64, wherein the purified peptide is introduced to cells through chemical formulations which destabilize cellular membranes.
   68. The cell of any one of paragraphs 62-67, wherein the cell is a fibroblast.
   69. The cell of paragraph 68, wherein the fibroblast is a human dermal fibroblast.
   70. The cell of any one of paragraphs 62-67, wherein the cell is a stem cell.
   71. The cell of paragraph 70, wherein the stem cell is selected from the group consisting of: hematopoietic stem cells, skeletal muscle stem cells, and mesenchymal stem cells.
   72. The cell of paragraph 70 or paragraph 71, wherein the stem cell is a human induced pluripotent stem cell.
   73. The cell of any one of paragraphs 62-67, wherein the cell is selected from the group consisting of: endothelial cells, chondrocytes, keratinocytes, and corneal epithelial cells.
   74. The cell of any one of paragraphs 62-73, wherein the cell expresses the protein at a level that is higher than a baseline level.
   75. A pharmaceutical composition comprising the cell of any one of paragraphs 62-74 and a pharmaceutically-acceptable excipient and/or polymeric carrier.
   76. A pharmaceutical composition comprising:
(a) a recombinant vector genome comprising one or more transgenes encoding one or more polypeptide sequences selected from ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IP05, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, and HSPA9,
wherein the vector genome is configured to express the one or more transgene at a level sufficient to induce tissue rejuvenation and/or regeneration; and
(b) a pharmaceutically-acceptable excipient and/or polymeric carrier.
   77. A method, comprising administering the pharmaceutical composition of paragraph 64 or 65 to skin of a subject via intraepidermal, transepidermal, intradermal, transdermal, subcutaneous, intramuscular, or topical administration.
   78. The method of paragraph 77, wherein the skin exhibits signs of aging.
   79. The method of paragraph 78, wherein the signs of aging are indicative of a skin condition selected from photoaging, intrinsic aging, aging induced by repeated facial expressions, loss of tone and gravity manifested in rough texture, sagging, wrinkles, furrows, folds, and any combinations thereof.
   80. A method for measuring the age of a cell, comprising:
(a) contacting a cell with a first reagent capable of recognizing cytoplasmic actin, with a second reagent capable of recognizing Golgi apparatus/apparati, with a third reagent capable of recognizing plasma membrane, with a fourth reagent capable of recognizing extranuclear DNA, with a fifth reagent capable of recognizing mitochondria, with a fifth reagent capable of recognizing endoplasmic reticulum, with a sixth reagent capable of recognizing RNA.
(b) determining the morphological and/or functional fingerprint of the cell based on the intensity of a signal associated with the first, second, third, fourth, fifth, and sixth binding reagent and the intensity of a signal associated with the at least second binding reagent;
(c) and identifying the age of the cell based upon a machine learning algorithm of extracted features.
   81. The method of paragraph 80, wherein the age of the cell is further measured using a machine learning algorithm of a cell's expression features in combination with morphological and/or functional features.
   82. A method for defining a rejuvenation index, called a ‘Youth score’, wherein the cell is contacted with a perturbant which induces a cell's transition from aged (reference) cell state to younger (altered) cell state, comprising:
(a) a change in a cell state, function and/or expression between the totality of unperturbed cells and the totality of perturbed cells; and
(b) comparing the cell's transition fingerprint thereby quantifying the cellular transition due to the perturbation.

### EXAMPLES

The data used to infer genetic aging network comes from a previous study¹⁴ and consists of bulk RNA sequencing (with a read depth of roughly 500,000 transcripts per individual) on dermal fibroblasts taken from the upper arm (on rare occasions, buttocks) of 133 individuals with ages ranging from 1 to 96 years. For each individual, the expression levels of 27,142 genes were recorded. Many of these genes are read at zero or negligible counts in many individuals, so we retained only the 5,138 genes which had nonzero reads in more than half the donors. For each of these genes, a time series was created from the data as follows: for an age with multiple donors, the transcript count values were simply averaged; for ages with no donors (which occur at ages 4, 27, 34-36, 38, 40, 48, 49, 53, 54, 58, 59, 65, 67, 72-74, 76, 77, 79, 81, 85, 93, and 95), transcript count values were linearly interpolated from the nearest ages with donor values). The time series show no correlation with gender (point biserial correlation) or passage number of the cells (Pearson correlation); due to the site of origin and racial composition of the donors (121/133 identified as white=91%), little-to-no variation was expected between ages in a time series based on race or difference in site-UV-exposure.

### Example 1. Inferring Novel Drivers

Using the processed transcriptome data, we sought to infer mechanistic interactions between genes which are essential to the aging process. Because the data represents a collection of time series, one for each gene transcript measured at each age, we assembled a battery of orthogonal tests for inferring mechanistic interactions between concurrently measured time series. Initially, we tested all ˜25 million possible gene-gene interactions using convergent cross mapping (CCM)¹⁵. CCM has previously been shown to be efficient at inferring mechanisms with sparse time-series data, for instance in ecological and physical systems^{16,17}. The method consists of a leave-one-out cross-validation test for Takens' theorem¹⁸, an observation that mechanistically coupled variables have the unusual property that data points clustered in a delay embedding (a plot of the points on the axes {X (t), X (t−1), X (t−2), . . . X (t−E−1)}) of one variable will also be clustered in the other variable.

CCM assigns to each possible gene-gene pair a score which is representative of interaction strength (or, confidence in the inferred interaction), and therefore does not offer binary yes/no interpretations on whether or not a given coupling exists between two genes. We therefore implemented our own such interpretation by keeping only the 150,000 highest end-library CCM scores, representing the top 0.6% of interactions (see Example 5). We then re-tested all the resulting interacting pairs found via CCM with an alternative method, transfer entropy¹⁹. Transfer entropy supported 91,222 of the top pairs via CCM, and we discarded the remaining ˜60,000.

We sought to classify the most important genes associated with changes in the transcriptome over time. While there are many possible metrics that would accomplish this goal, we sought something simple and relatively robust against the various sources of noise in the data. To that end, we assigned each gene i a master score Mᵢ based on the amount of “reliable” change observed in the gene's own transcription time series, as well as the amount of change in every gene immediately downstream of it in the inferred network. This was calculated according to Formula 1:

$${M}_{i} = {S}_{i} ( 1 - {p}_{i} ) {\sum }_{j} {Δ}_{i j} \vert {S}_{j} \vert ( 1 - {p}_{j} ) , \qquad Formula 1$$

where Sᵢ is the slope of a line fit to the time series of gene i, pᵢ is the p-value resulting from the Spearman correlation between the set of ages and the time series (to measure how monotonically the expression of gene i is increasing/decreasing with time), and Δᵢⱼ is the adjacency matrix for the inferred network.

### Example 2. Experimental Design and Gene Library Construction Based on Inferred Genes and Controls from the Literature

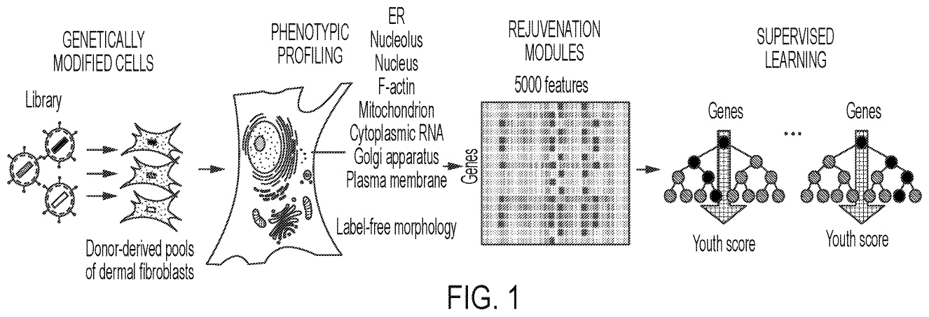

From the score imputed to each gene according to the above calculation, we selected the top 150 genes to move forward with experimental validation, as well as 25 control genes. As shown in FIG. 1, the experimental design consists of overexpressing each of these 175 genes in dermal fibroblasts drawn from a pool of six 55-year-old donors. Dermal fibroblasts from two donor pools (55yo and 25yo) were transduced with individual members from the genetic library combining novel inferred drivers of aging and controls from the literature. Hold-out populations from both pools were subjected to transduction protocols but received no virus, to serve as untreated controls. Next, the CellPaint assay assess thousands of features relevant to cell morphology and cell physiology. These features were then digested in a machine learning framework to identify which members from the genetic library successfully shifted the overall cell phenotype from the 55yo-untreated population towards the 25yo-untreated population. Overexpression is accomplished via transduction of cells with a transgene consisting of a promoter and ORF corresponding to the library member, with lentivirus as the vector. As points of comparison, a subpopulation of the 55yo cells undergo transduction protocols, but do not receive virus—these are called ‘untreated’, since they have undergone the same cellular stresses as the transduced cells but have not received a transgene. Additionally, a second pool consisting of six 25-year-old donors is similarly subjected to transduction-without-virus protocols. Transduced and control cells are then examined via various assays whose ultimate aim is to assess both molecular and functional similarities between transduced 55yo cells and the untreated 55yo and 25yo cells; a gene is considered a successful rejuvenative target if transduced cells become more similar to 25yo cells than untreated 55yo cells.

After assembling the library and transducing cells, quality control measures eliminated 18 library members (10 control genes and 8 novel targets). These eliminations could be due to the transgene being too large for the lentiviral vector, failure of viral assembly, failure during transduction protocols, or other reasons. The library members that moved forward for assays are listed in Table 1.

TABLE 1
De-anonymization of library genes. Ordering is by Youth Score (‘YScore’),
in descending order. Control genes follow below breakout double lines.

| Collection ID | Gene Name | YScore(mean +/− std) | ROS(mean +/− std), p | Count(mean +/− std), p |
|---|---|---|---|---|
| YoungNT | YoungNT | 0.83 +/− 0.01 | 5.21 +/− 1.42 | 338 +/− 168 |
| 14 | ANXA2 | 0.80 +/− 0.02 | 4.62 +/− 1.16, p = 0.706 | 191 +/− 109, p = 0.678 |
| 110 | PRRC2B | 0.78 +/− 0.03 | 3.86 +/− 1.14, p = 0.347 | 340 +/− 105, p = 0.077 |
| 68 | MAP4 | 0.78 +/− 0.01 | 3.71 +/− 1.48, p = 0.315 | 288 +/− 63.2, p = 0.118 |
| 135 | BGN | 0.76 +/− 0.03 | 3.99 +/− 1.02, p = 0.393 | 375 +/− 120, p = 0.049 |
| 42 | CLTA | 0.73 +/− 0.02 | 3.70 +/− 1.29, p = 0.301 | 328 +/− 118, p = 0.129 |
| 103 | RPS23 | 0.73 +/− 0.02 | 4.63 +/− 0.57, p = 0.698 | 257 +/− 159, p = 0.641 |
| 154 | AKT1 | 0.69 +/− 0.02 | 4.20 +/− 2.23, p = 0.559 | 310 +/− 43.1, p = 0.025 |
| 40 | VCP | 0.66 +/− 0.02 | 4.15 +/− 1.61, p = 0.491 | 285 +/− 70.4, p = 0.154 |
| 65 | FAM129B | 0.65 +/− 0.01 | 4.71 +/− 1.97, p = 0.784 | 183 +/− 43.4, p = 0.324 |
| 74 | LMAN1 | 0.65 +/− 0.01 | 3.97 +/− 1.12, p = 0.389 | 181 +/− 41.3, p = 0.299 |
| 64 | GUK1 | 0.64 +/− 0.02 | 3.91 +/− 1.68, p = 0.398 | 267 +/− 63.6, p = 0.245 |
| 44 | SEC61A1 | 0.64 +/− 0.01 | 3.67 +/− 1.8, p = 0.323 | 326 +/− 96.1, p = 0.086 |
| 29 | ELL2 | 0.61 +/− 0.01 | 5.71 +/− 2.44, p = 0.710 | 85.8 +/− 17.5, p = 0.002 |
| 127 | RPN1 | 0.61 +/− 0.03 | 4.03 +/− 1.79, p = 0.455 | 332 +/− 113, p = 0.109 |
| 105 | KIF5B | 0.61 +/− 0.01 | 4.77 +/− 1.59, p = 0.804 | 186 +/− 23.6, p = 0.283 |
| 100 | MSN | 0.60 +/− 0.03 | 4.33 +/− 0.90, p = 0.540 | 228 +/− 45.4, p = 0.726 |
| 125 | TGOLN2 | 0.60 +/− 0.04 | 5.64 +/− 1.65, p = 0.705 | 73.4 +/− 51.6, p = 0.006 |
| 170 | NRF2 | 0.59 +/− 0.07 | 7.45 +/− 4.92, p = 0.339 | 88.6 +/− 54.1, p = 0.011 |
| 168 | SOD2 | 0.55 +/− 0.03 | 7.35 +/− 4.30, p = 0.314 | 93.2 +/− 7.10, p = 0.002 |
| 171 | MSRA | 0.43 +/− 0.02 | 4.76 +/− 1.98, p = 0.812 | 250 +/− 48.6, p = 0.355 |
| 166 | TERT | 0.39 +/− 0.01 | 5.07 +/− 1.85, p = 0.973 | 192 +/− 13.9, p = 0.338 |
| 174 | DAP | 0.33 +/− 0.00 | 7.29 +/− 4.19, p = 0.321 | 82.5 +/− 10.9, p = 0.001 |
| 161 | LAMB3 | 0.29 +/− 0.01 | 4.51 +/− 1.90, p = 0.677 | 238 +/− 41.2, p = 0.531 |
| 163 | SIRT1 | 0.23 +/− 0.03 | 3.92 +/− 0.98, p = 0.361 | 164 +/− 28.0, p = 0.095 |
| 173 | ATG5 | 0.23 +/− 0.02 | 6.42 +/− 3.48, p = 0.496 | 170 +/− 31.2, p = 0.143 |
| 165 | FGF21 | 0.21 +/− 0.02 | 6.69 +/− 3.27, p = 0.396 | 169 +/− 58.4, p = 0.251 |
| 167 | FOXO3 | 0.18 +/− 0.02 | 6.00 +/− 2.95, p = 0.612 | 194 +/− 41.2, p = 0.487 |
| OldNT | OldNT | 0.15 +/− 0.01 | 5.11 +/− 2.90 | 217 +/− 46.0 |
| 175 | SIRT6 | 0.15 +/− 0.03 | 8.18 +/− 4.33, p = 0.179 | 32 +/− 26.4, p = 0.0004 |
| 162 | COL3A | 0.15 +/− 0.01 | 6.04 +/− 3.98, p = 0.656 | 148 +/− 16.7, p = 0.032 |
| 169 | UCP1 | 0.12 +/− 0.01 | 5.17 +/− 2.54, p = 0.973 | 129 +/− 28.4, p = 0.017 |
| 176 | MeCP2 | 0.04 +/− 0.01 | 7.20 +/− 5.83, p = 0.451 | 142 +/− 75.3, p = 0.141 |

The control genes that moved forward for assays are NRF2²⁰, a transcription factor with known regulatory impact on cellular metabolism; SOD2²¹, a gene whose deficiency causes senescence due to mitochondrial failure; MSRA²², a gene with known mitochondrial function that decreases the rate of age-related diseases in mice; TERT²³, telomerase reverse transcriptase; LAMB3, a critical extracellular matrix component; SIRT1²⁴ and SIRT6²⁵, NAD+-dependent enzymes known to regulate many signaling processes implicated in aging; ATG5²⁶, a gene necessary to the creation of autophagosomes, whose overexpression extends murine lifespans; FGF21²⁷, a cytokine which is overexpressed during fasting and which extends the lifespan of food-deprived mice; FOX03²⁸, a homeostasis regulator implicated in many age-related diseases; COLUA, a critical extracellular matrix component and frequent therapeutic target; UCP1²⁹, a gene related to mitochondrial function in adipose tissue which therefore couples adiposity, metabolism, and insulin signaling pathways; and two negative controls: MeCP2³⁰, a silencing factor which is strongly associated with senescence, and DAP³¹, a negative regulator of autophagy, a pathway whose dysfunction is thought to be a hallmark of aging.

### Example 3. CellPaint Morphological Assay

As an initial assessment of the effects of the genes in our library, we subjected primary dermal fibroblasts pooled from six 55yo donors and six 25yo donors to a novel assay, FIG. 1, which uses five fluorescent channels specific to the structure and activity of five classes of organelles: Hoechst 33342 (DNA), concanavalin A (endoplasmic reticulum), SYTO 14 (nucleoli and cytoplasmic RNA), phalloidin (actin) and WGA (Golgi and plasma membrane), and MitoTracker Deep Red (mitochondria). This assay has previously been used to examine cell phenotypes and cellular responses to drugs^{32, 33}, and can extract thousands of features related not only to the basic shape and size of cellular and sub-cellular components, but also the various correlations and interactions between these components, thereby comprising most features of interest in cell physiology and morphology.

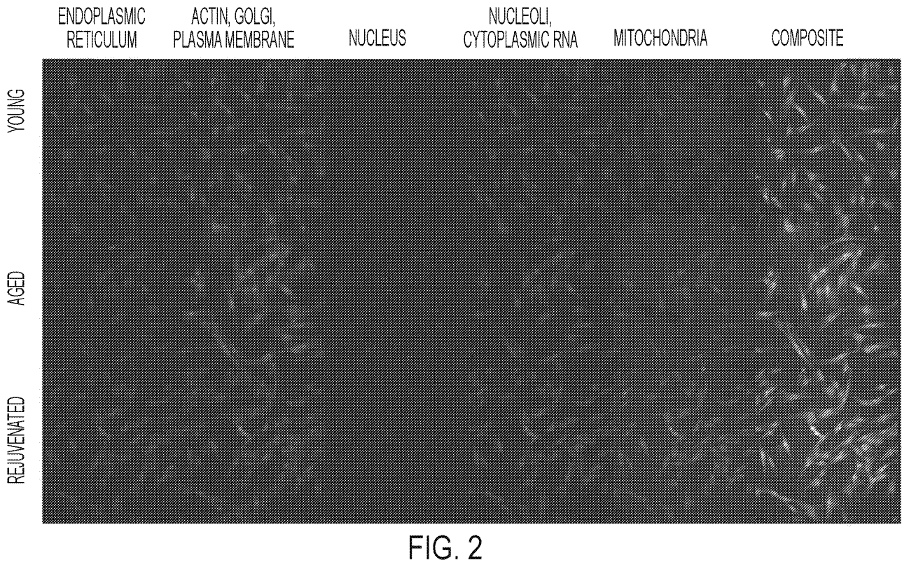

Subjecting untreated 25yo and 55yo cells to our transduction protocols (except the actual step of virus transduction) and including them in the CellPaint assay allows us to identify the morphological features that distinguish between young and old cells. These include known shape changes in aged cells, including loss of aspect ratio, increase in cell size, and increase in nucleus size³⁴, as well as differences in mitochondrial and ER activity. An example of the visual differences between the two age groups can be seen in FIG. 2. As exemplified in FIG. 2, CellPaint is an imaging technique utilizing five stained channels to identify markers related to the endoplasmic reticulum (ER), actin/Golgi/plasma membrane (AGP), DNA, RNA, and mitochondria. This example shows all five channels (and their combination) for dermal fibroblasts taken from a pool of 25yo donors (top row), a pool of 55yo donors (middle row), and the same 55yo cells transduced with one particular gene (Gene110).

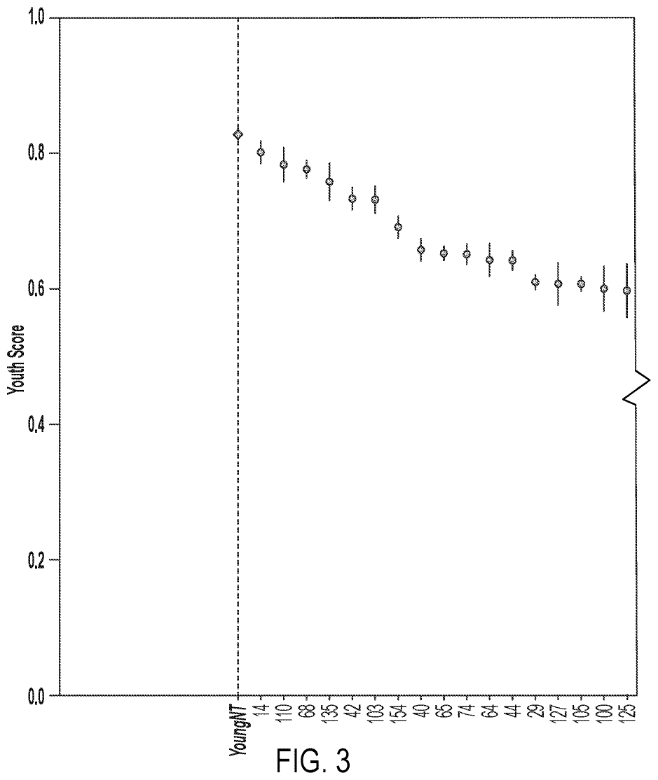

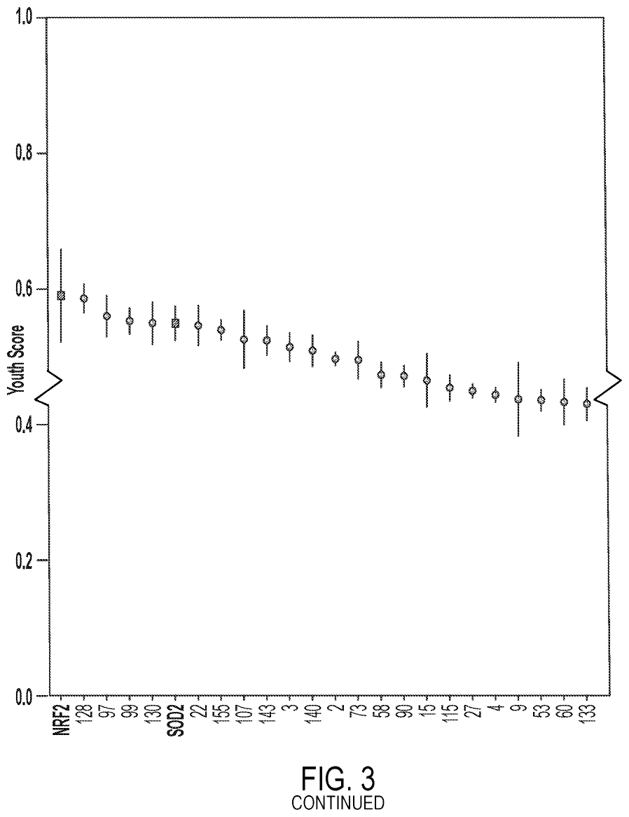

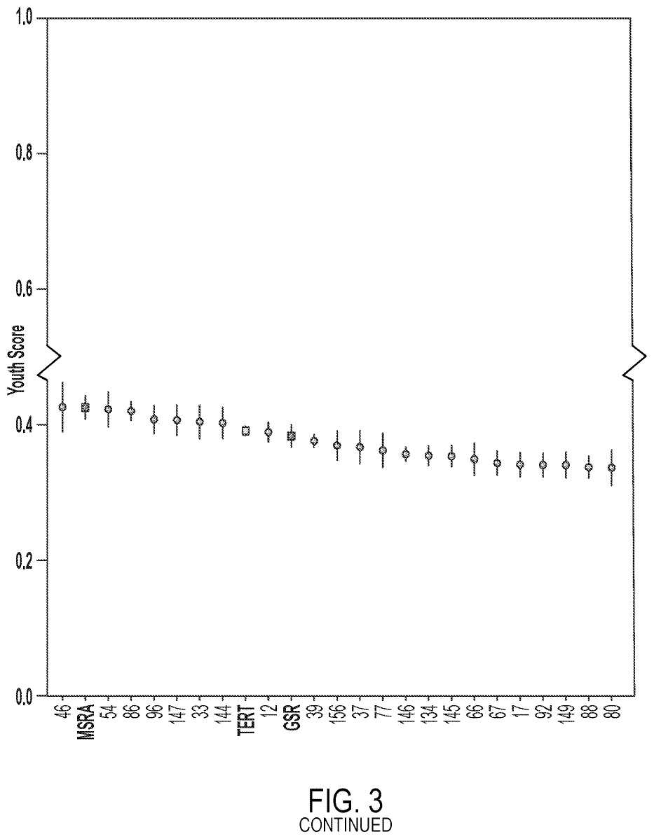

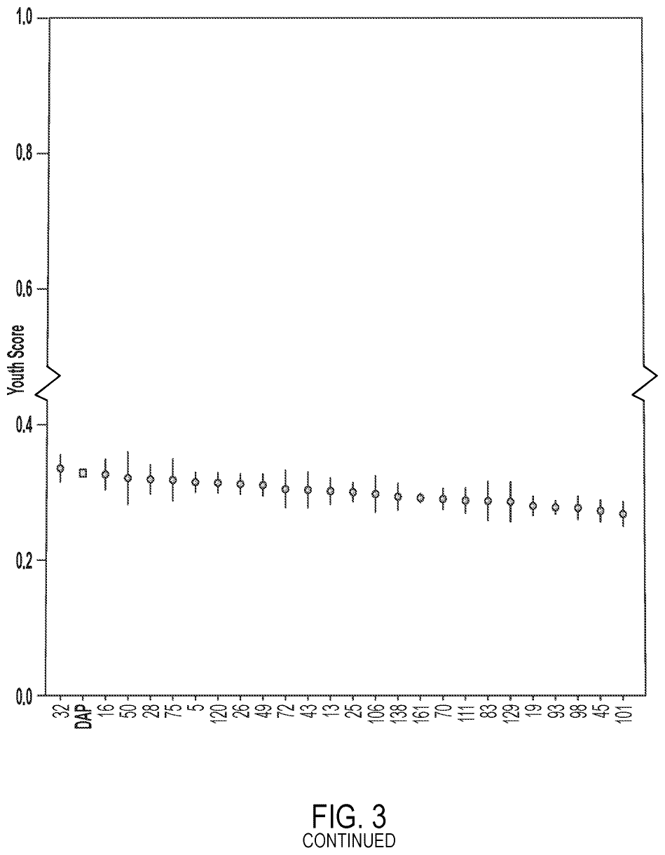

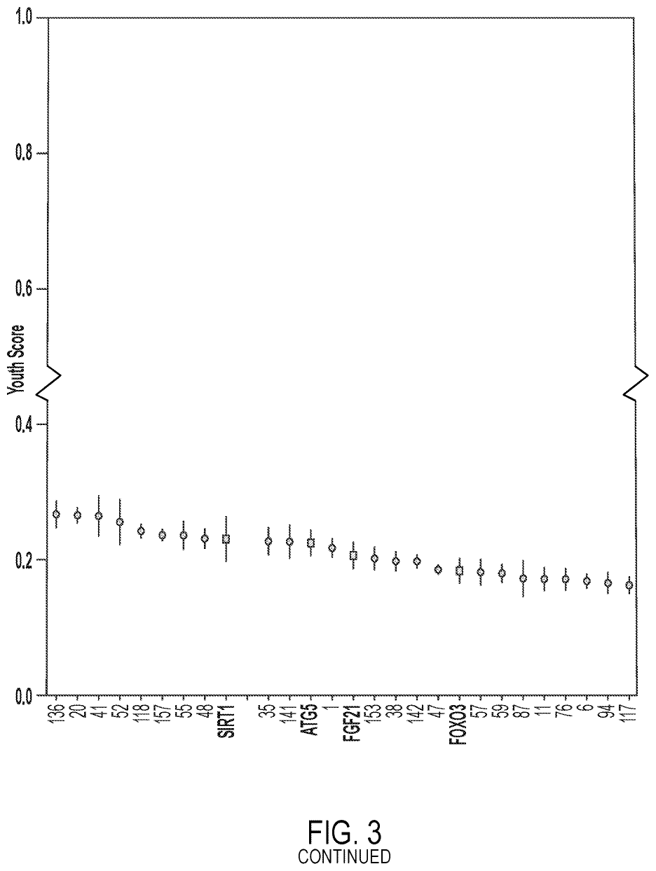

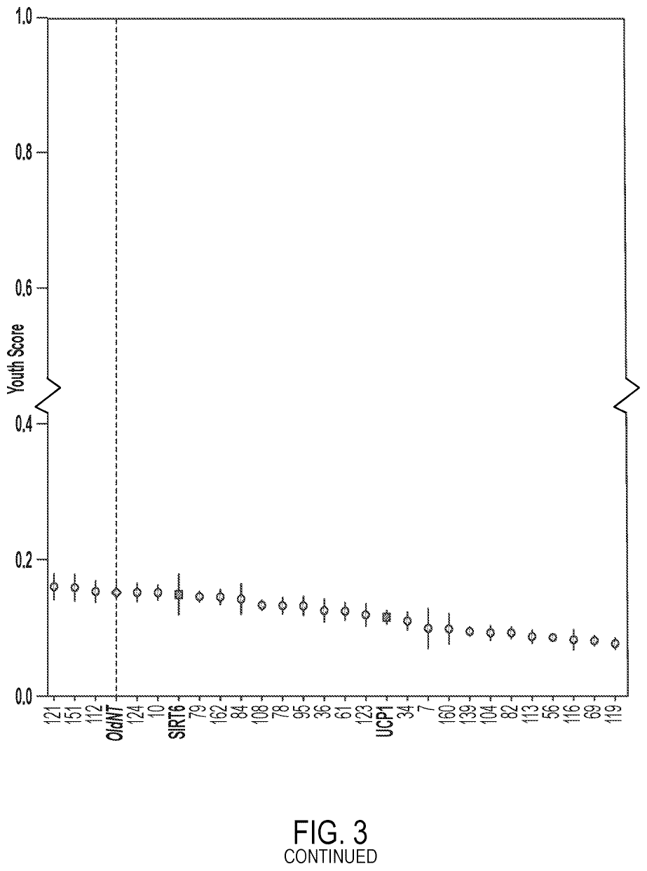

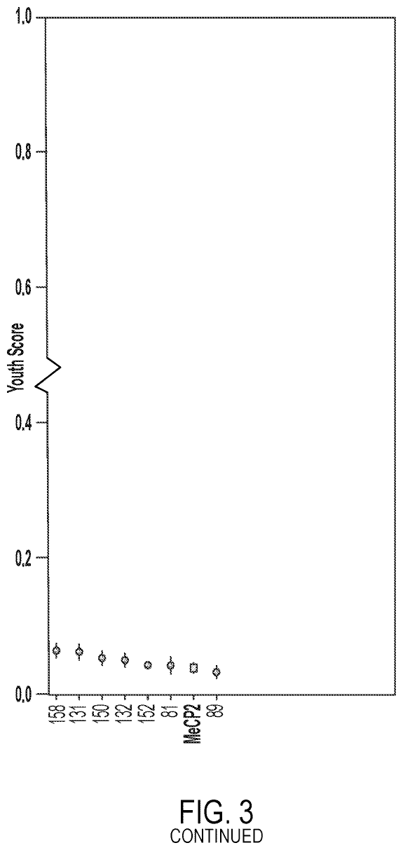

To more rigorously understand the morphological difference between 25yo and 55yo cells, we used supervised machine learning (see Example 5) to train a classifier to distinguish between the two age groups. Our classifier was able to achieve an accuracy of 83.1±0.5% on validation hold-out sets and bases its classifications on a very small fraction of the available features from the CellPaint assay (18 out of ˜2000). We then classified each 55yo cell which had been transduced by a gene from our library—if the gene has no effect, then the cell should be correctly classified as 55yo, whereas sufficient rejuvenative effects could lead a cell to be misclassified as 25yo despite originally coming from pooled 55yo donors. We call the fraction of cells transduced with a particular gene which are misclassified as 25yo (i.e., the rate of false negatives) the “youth score”, which is plotted for the library and controls in FIG. 3. FIG. 3 shows The CellPaint assay returns a large amount of data; in total, around 5,000 features pertaining to cell morphology (shape, size) and physiology (organelle distribution and behavior) are measured on each single cell. After various quality control measures, we kept an identical set of ˜2,000 features for each cell and trained a binary machine learning classifier (see Example 5) on 25yo cells (YoungNT) and 55yo cells (OldNT) which underwent virus transduction protocols but received no transgene (hence ‘untreated’). The classifier can identify 25yo and 55yo cells with high accuracy in a blind validation set (83.1%±0.5%) based on a much smaller set of features. We then applied the classifier to every single 55yo cell transduced with a library member. We call the false-negative rate (that is, the fraction of single cells which are identified as 25yo despite originating from 55yo donors) the ‘Youth Score’ for that particular library member. Here the Youth Score is plotted in descending order. Controls from the literature are marked in red. Many novel genes outperform all control genes. Novel genes, anonymized by numbers, are identified in Table 1.

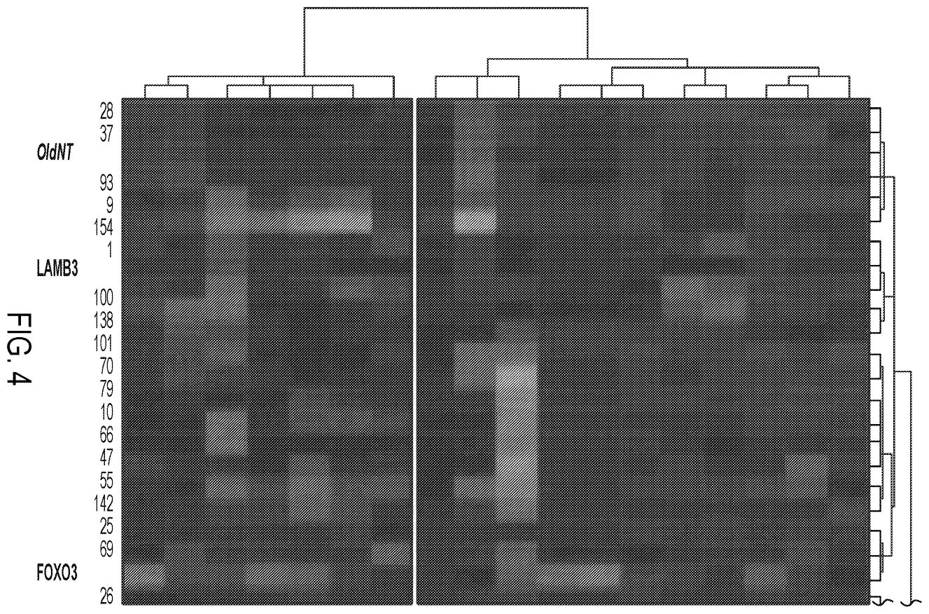

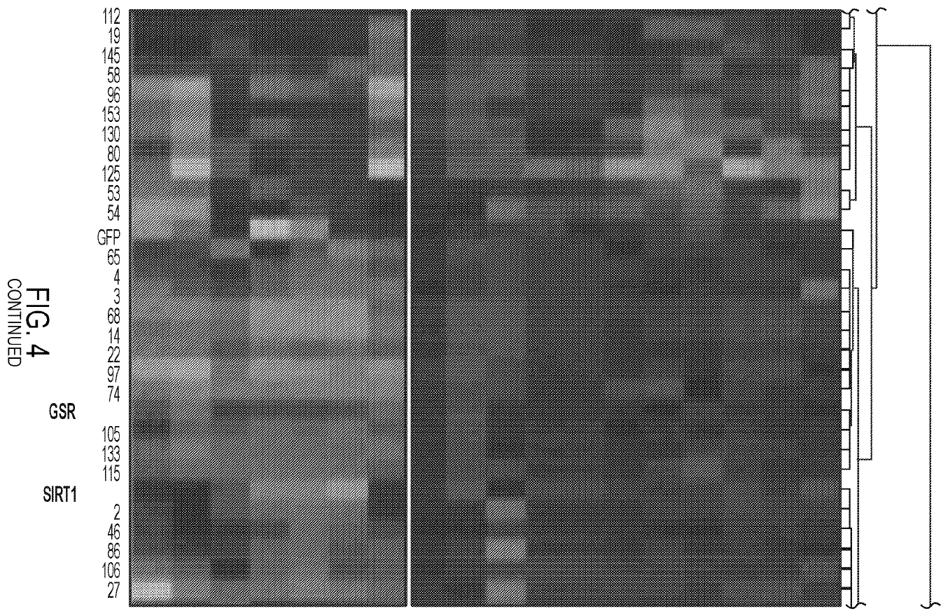

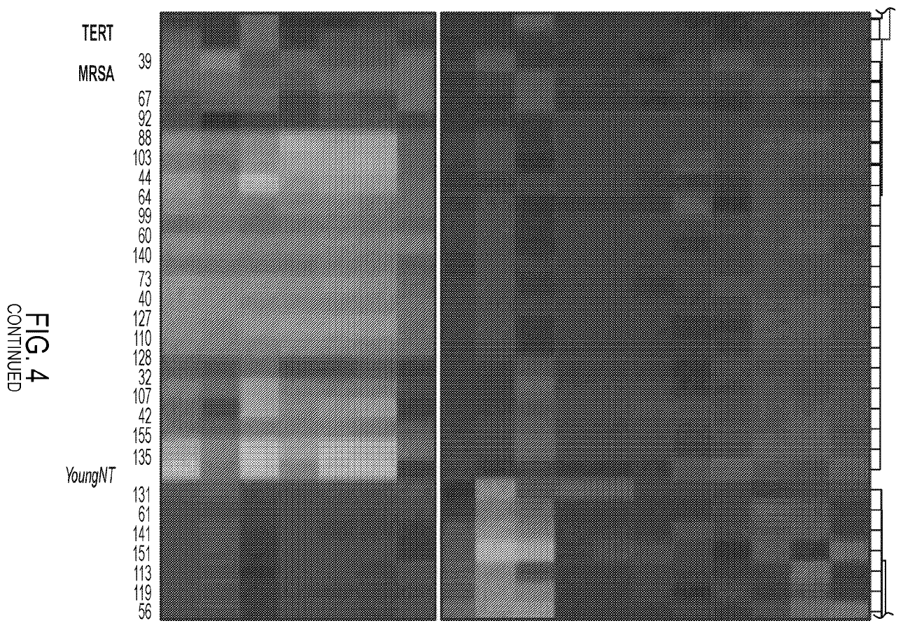

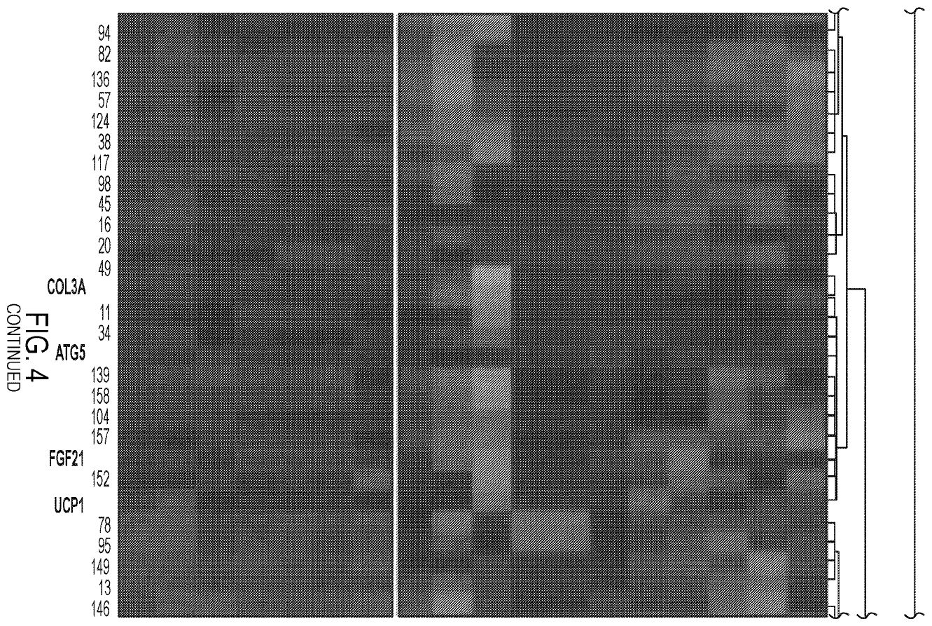

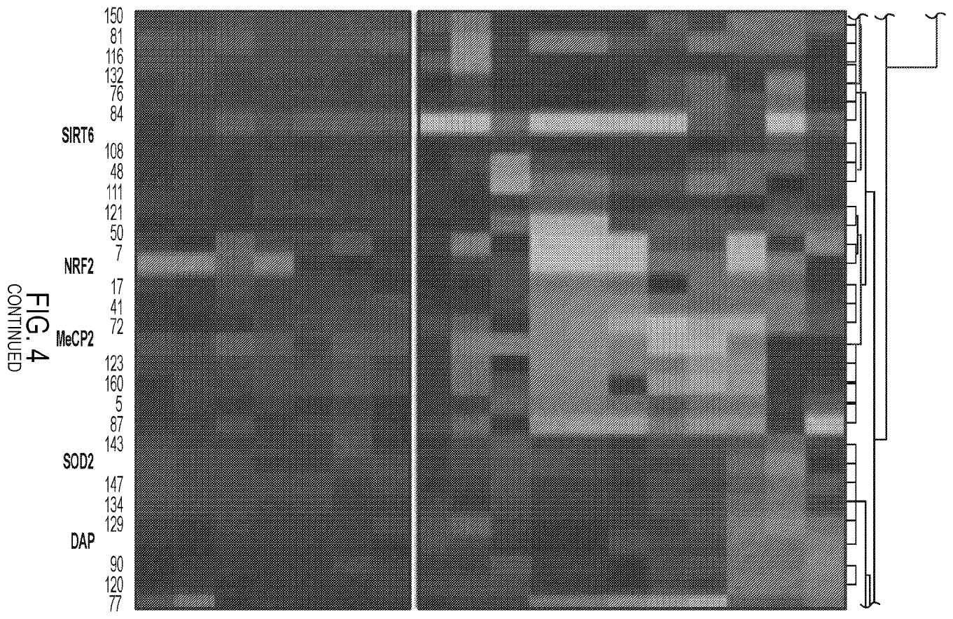

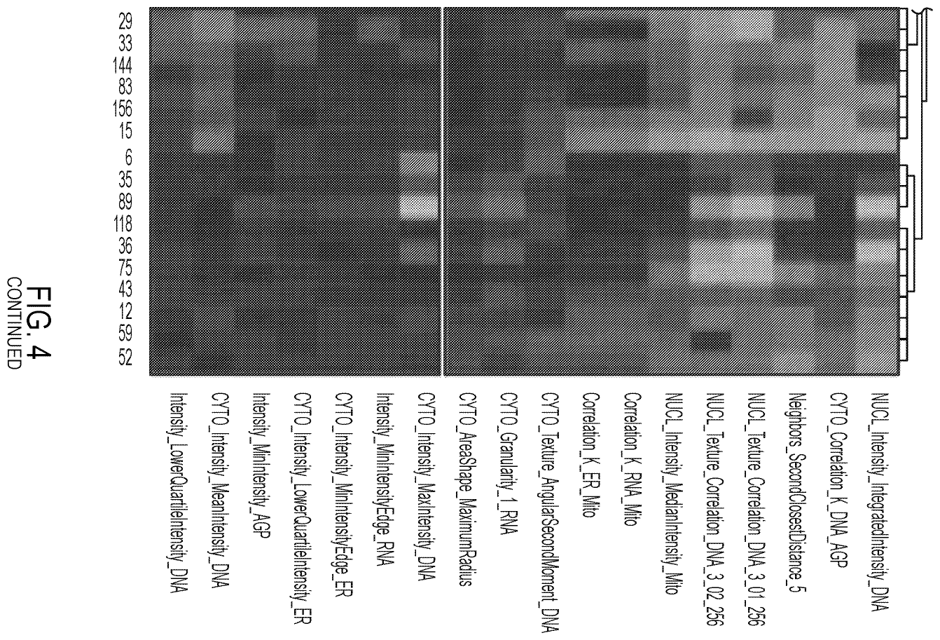

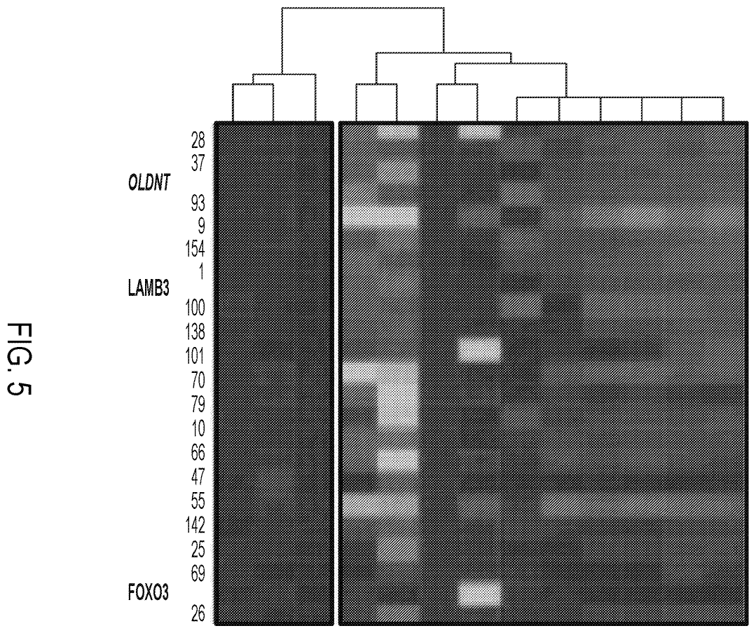

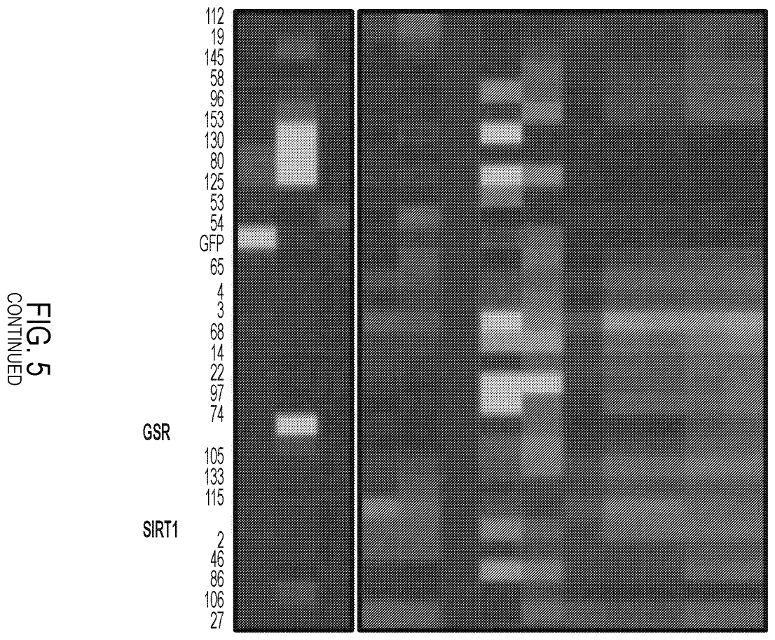

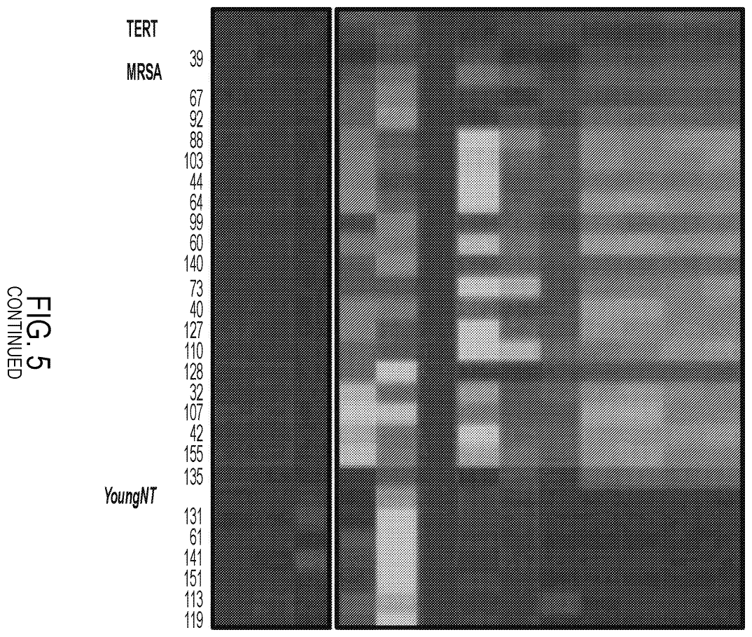

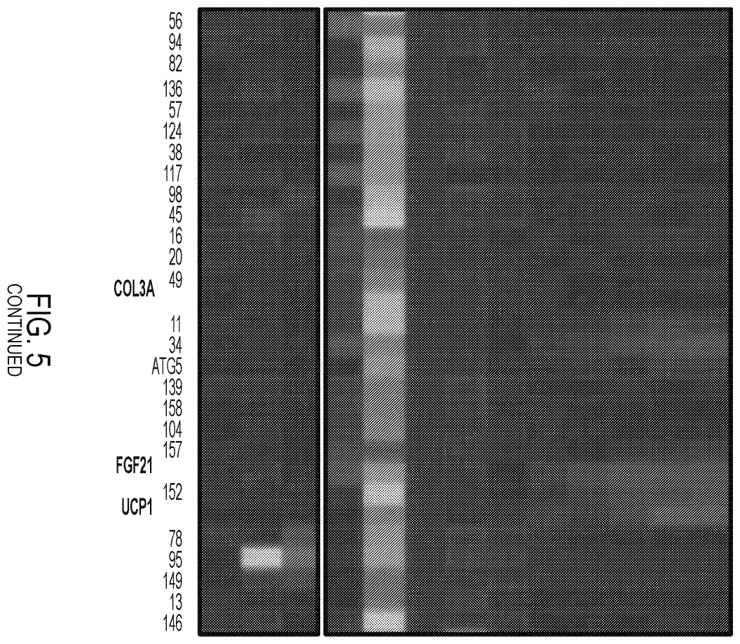

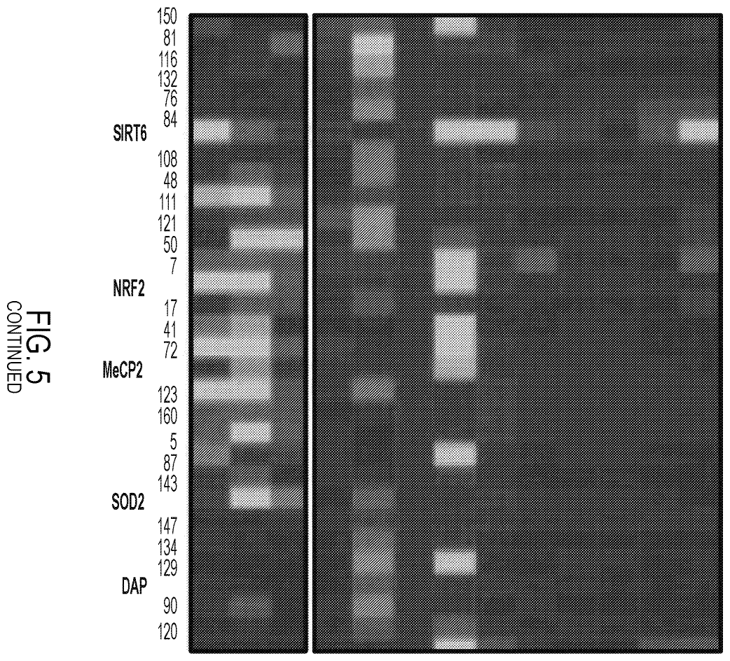

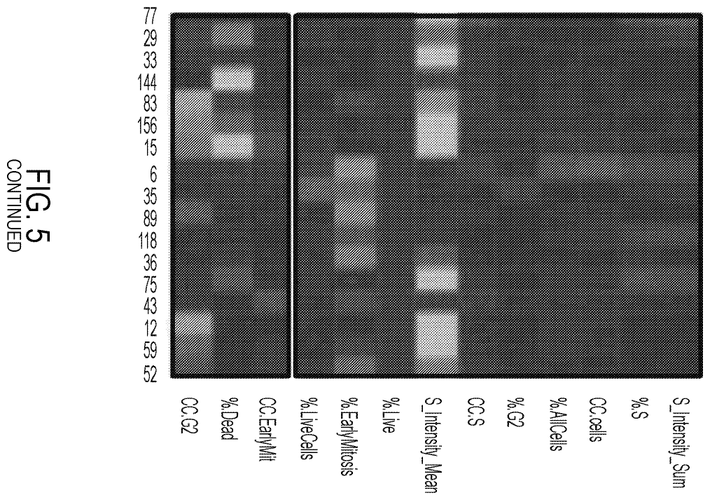

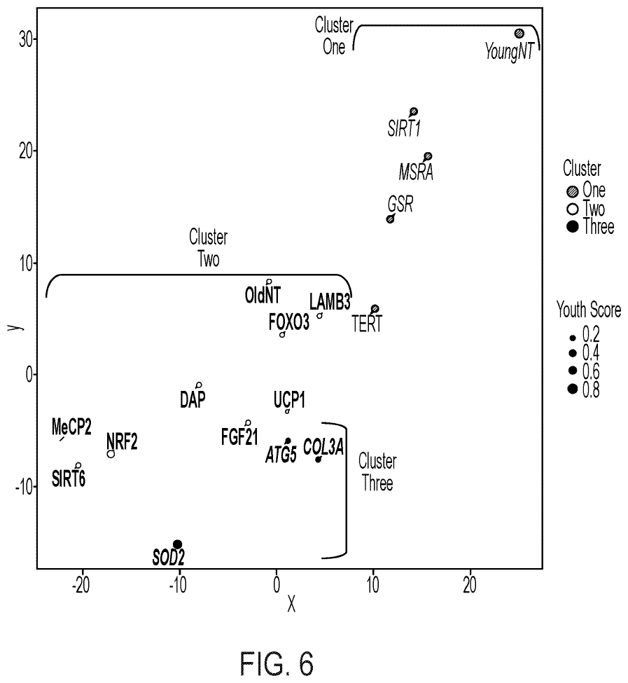

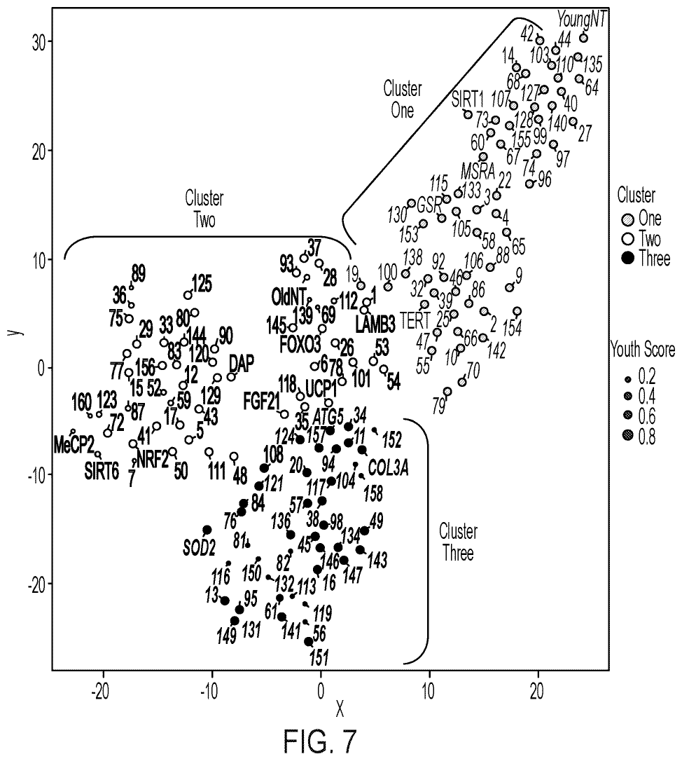

While most of the genes elected to our library via our inference method led to high youth scores, some were especially successful, indicating high rejuvenative potential. Most of the top-performing genes in the library according to youth score recapitulated the youthful value of nearly all of the 18 features included in the machine learning models; in contrast, many positive controls seem to have “off-target” effects, recapitulating a part of the rejuvenative fingerprint while diverging from it in other key aspects (FIG. 4). FIG. 4 shows a hierarchically clustered heatmap of the 18 features used by the machine-learning classifier to distinguish between 25yo and 55yo untreated cells for every library member and control gene. Novel genetic targets with high youth score tend to cluster with 25yo untreated cells (‘YoungNT’) across all features. Many positive control genes, on the other hand, are only similar to 25yo untreated cells across a few features—therefore insofar as these genes have rejuvenative effects, there seem to be “off-target” effects as well. For instance, many of the positive controls implicated in mitochondria and metabolism (SOD2, SIRT6, NRF2) show off-target metabolic effects (intensity of mitochondria channel and correlation between mitochondrial and non-mitochondrial intensities). Cell cycle profiles data of healthy 25yo, 55yo, and treated is shown in FIG. 5. We observed a consistence functional rescue of growth rate without disturbance in cell cycle. Interestingly, except for SIRT1, all control targets do not rescue cell growth, while our top performing genes consistently rescue life span. A final t-SNE embedding of the CellPaint data is shown in FIGS. 6-7, showing that the library members with high Youth Score also cluster with young untreated cells based on bare feature values. Specifically, FIG. 6 shows that a further dimensional reduction can be achieved via t-SNE, an algorithm which projects points to a lower dimensional space for better visualization. The 18 features shown in FIG. 4 have been projected down to two dimensions. A k-means clustering algorithm has been applied to assign points their color (cluster), whereas point size is proportional to the library member's Youth Score. The majority of the genes cluster closest to untreated 55yo cells (OldNT). FIG. 7 shows that further dimensional reduction can be achieved via t-SNE, an algorithm which projects points to a lower-dimensional space for better visualization. The 18 features shown in FIG. 4 have been projected down to two dimensions. A k-means clustering algorithm has been applied to assign points their color (cluster), whereas point size is proportional to the library member's Youth Score. Consistent with earlier results, high-performing novel genes cluster closest to untreated 25yo cells (YoungNT).

### Example 4. Functional Assays Correlate with Youth Score

Next, we sought to balance the cell physiology and morphology assay (form) with measures of cell health and viability (function). In particular, we sought to validate a basic concept—that recapitulating a youthful cell phenotype, in a physiological sense, also restores basic functional properties which are associated with a young cell.

We applied functional measures of varying complexity to the library-transduced fibroblasts. At the simplest end, we evaluated two one-dimensional metrics of cell health: the abundance of reactive oxygen species (ROS), as well as the growth rate. For ROS abundance, we applied ThermoFisher's CellROX™ Deep Red Reagent to cells from each library member plated in wells with technical variates; the technical variates allow for the error bars on ROS abundance in FIG. 8. Lower frequencies of ROS, a class of deleterious molecules, are commonly associated with increased cell health—moreover, ROS has often been reported to increase with age. FIG. 8 shows the normalized frequencies of ROS measured in transduced cells versus the library members, organized by Youth Score in descending order. The high-performing novel genes according to the CellPaint assay are therefore seen to also have much lower ROS than other controls or library members. To normalize the ROS abundance across the wells, cells were co-stained with Hoechst 33342. Normalized abundances are plotted in FIG. 8. We see a strong concordance between the ROS assay and the CellPaint assay; higher youth scores correlate negatively with higher ROS abundance (p=−0.46, p=2×10⁻⁹ by Pearson correlation). The novel genes with exceptionally high youth scores also have exceptionally low ROS abundance compared to the rest of the library.

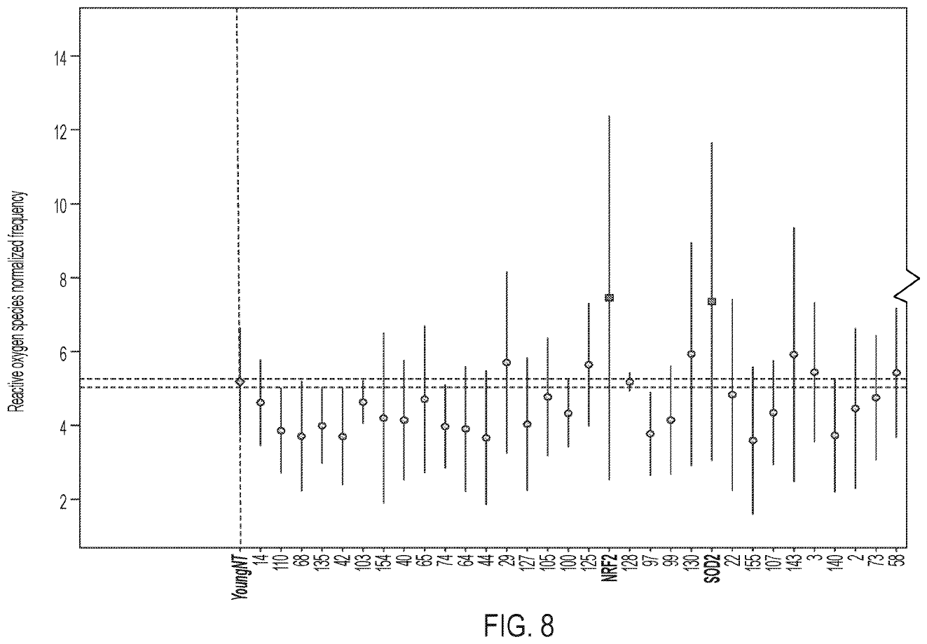

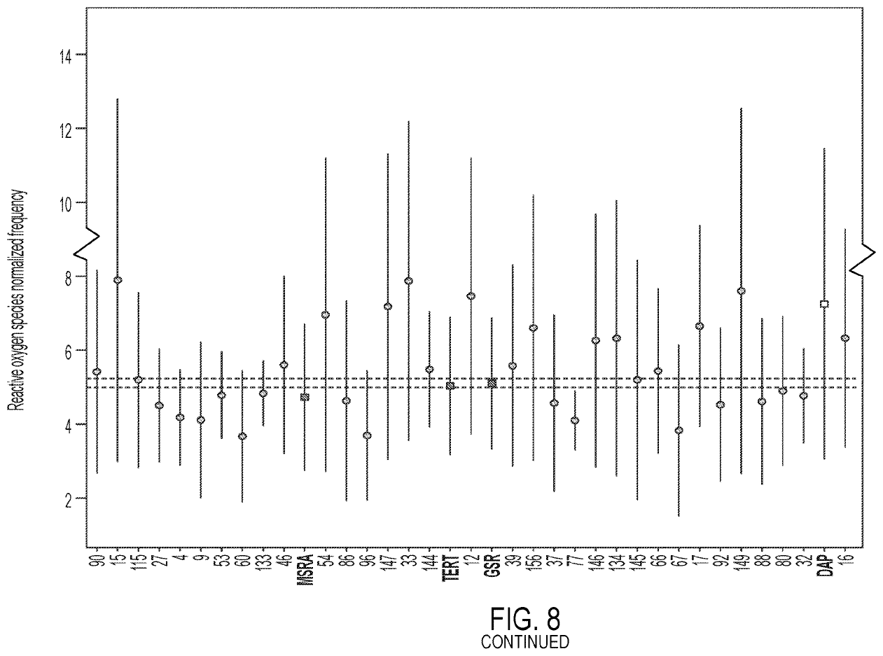

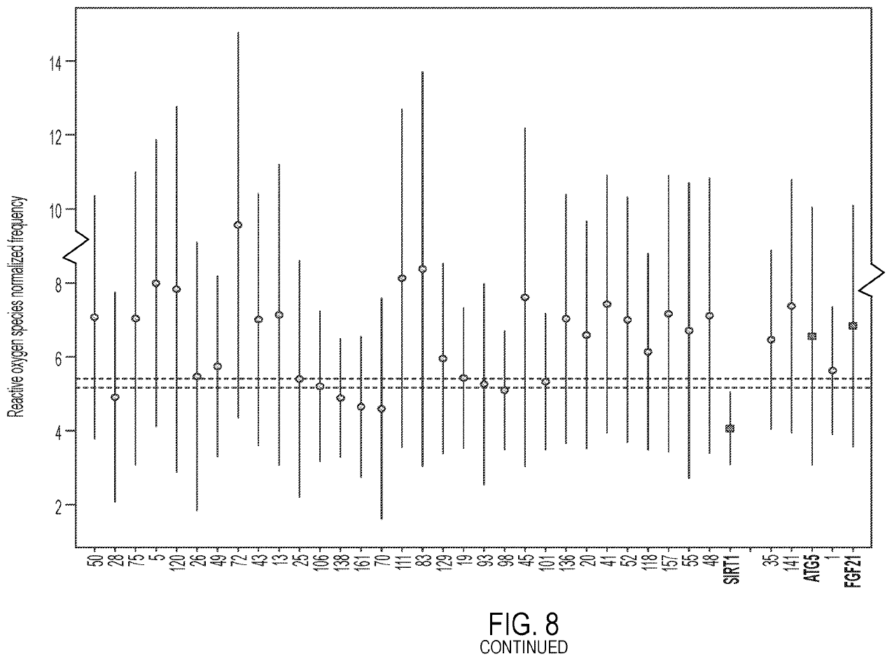

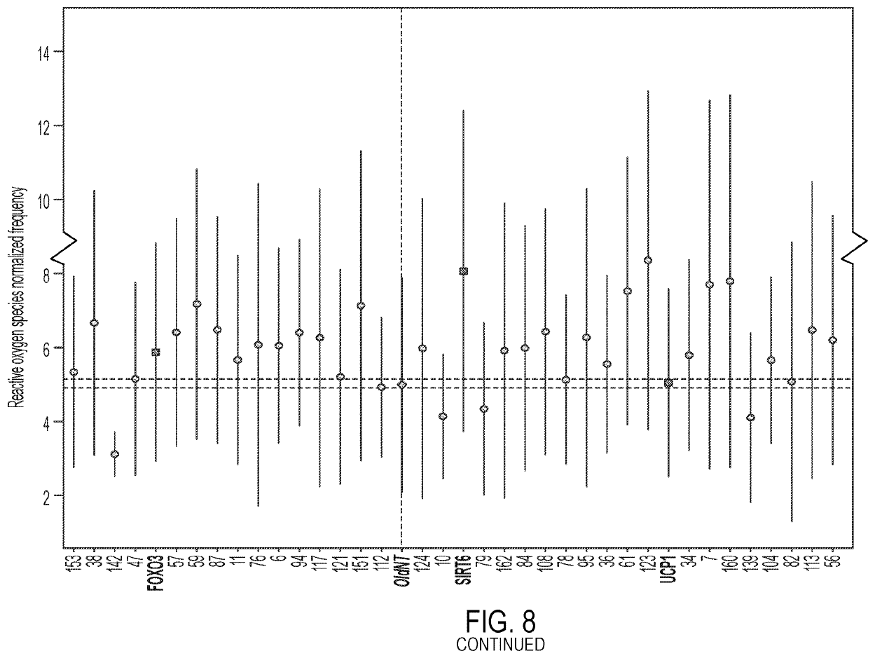

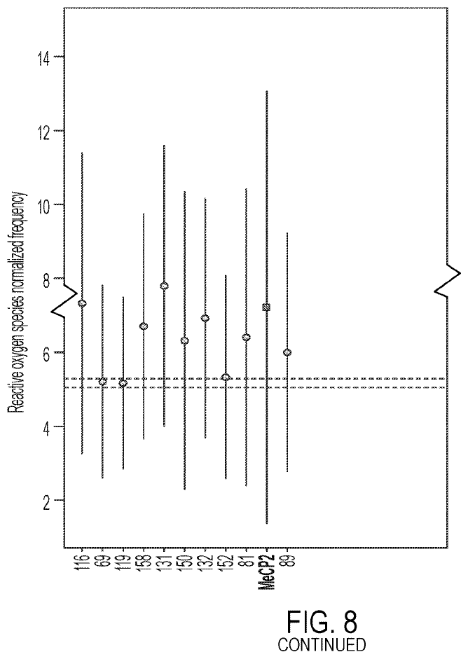

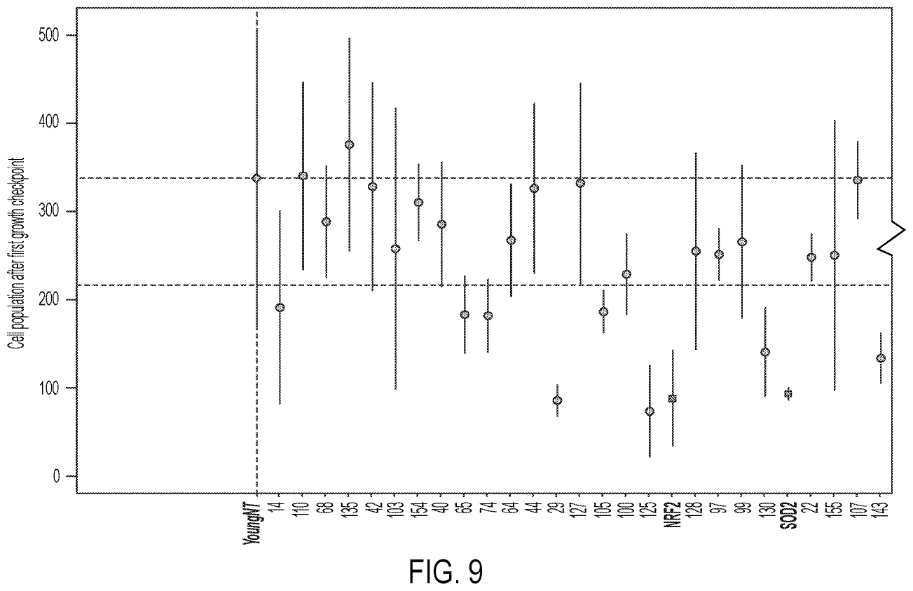

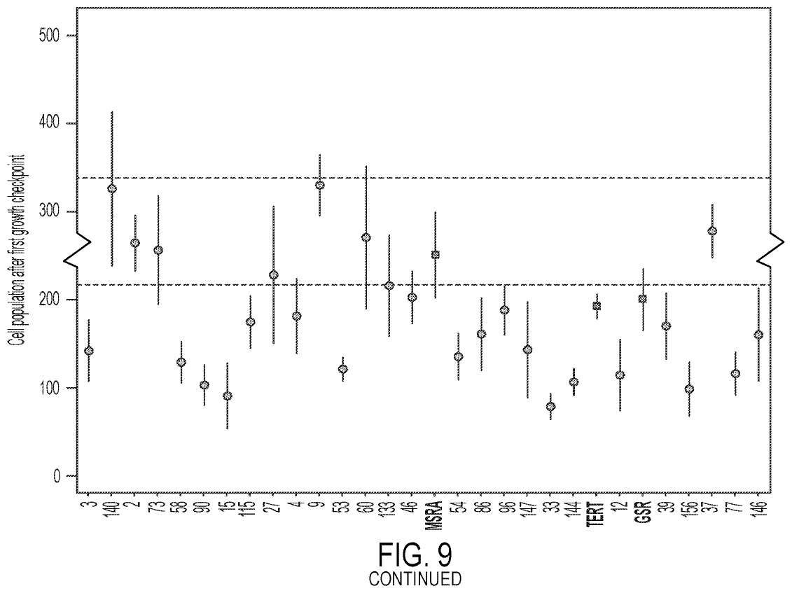

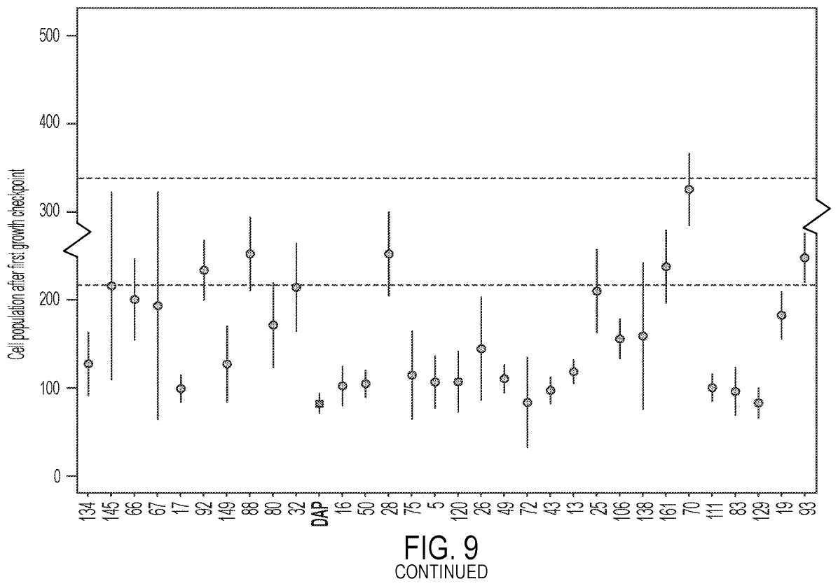

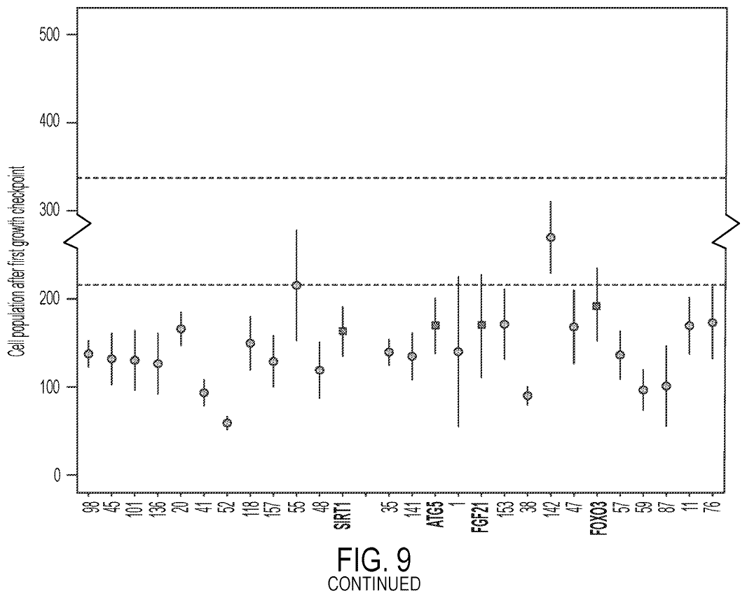

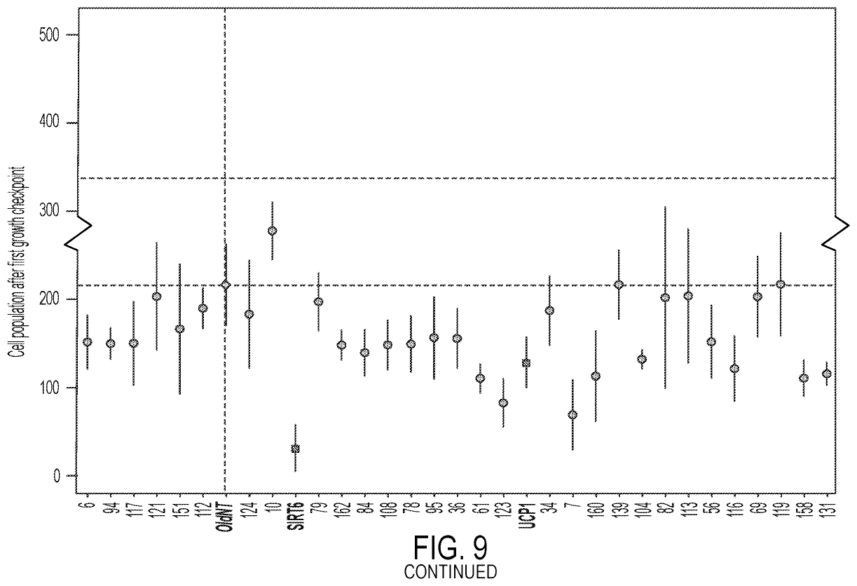

In place of a continuous growth rate, we calculated a proxy measure of replicative potential (FIG. 9). Without wishing to be bound by any theory, it is thought that aging cells from most tissues have lower replication rates than their younger counterparts. FIG. 9 therefore shows the populations of each library member in plate wells at the time the CellPaint assay was performed. Since each well is prepared with a nearly identical number of transduced cells a number of days before the CellPaint assay, this population size is a useful proxy measure for a continuous growth rate. Here, the mean well population size is plotted versus library members organized by descending Youth Score. The high-performing novel genes often have much higher growth rates than other library members. We measured the cell count in individual wells transduced with library members at the time the CellPaint assay was performed—since each well had been seeded with nearly equal numbers of cells-per-well days prior to the assay, this is a two-time-point approximation of the growth rate. By this measure, library candidates that lead to higher youth scores also replicated faster, as shown in FIG. 8. Similar to ROS abundance, we found that high youth score is correlated with high growth rates (p=0.37, p=2×10⁻⁶ by Pearson correlation). The concordance between these assays speaks both to the ability of the CellPaint assay and its features to be predictive of cell health, and to the robustness of the top-performing novel genes in their rejuvenative potential.

### Example 5. Methods Related to Examples 1-4

### Convergent Cross-Mapping (CCM)

For each of the measured 5, 138 genes, we computed the interaction strength with all other genes via Sugihara et al.'s¹⁵ convergent cross-mapping (CCM) approach. CCM accomplishes this by transforming an important theorem in dynamical systems theory into a leave-one-out cross-validation test for mechanistic coupling. Takens' theorem¹⁸ states that if two variables X and Y are coupled in a dynamical system, they have the property that, when embedded in the variables (X (t), X (t−1), . . . X (t−E)) (a delay embedding of order E; same embedding for Y, so that both variables are embedded completely independently of one another), local neighborhoods are preserved—meaning if the nearest neighbors in the X embedding of observation X(21) are {X(3), X(7), X(192)}, then if X and Y are coupled, the nearest neighbors of Y(21) should be {Y(3), Y(7), Y(192)}. This can be rigorously tested via the following outline:

1. Create the order-E delay embedding of X(t) and Y(t) (all possible E are tested), using a portion L* of the total length of the time series (L).
2. To test the idea of a mechanistic coupling X→Y, discard a single observation X(t*) (because if X→Y, it is Y that contains information about X and not the converse); the discarded datum will be approximated using data from Y).
3. The E+1 nearest neighbors of Y(t*), {Y (t₁), Y (t₂), . . . Y (t_{E+1})} are identified (because E+1 observations are necessary to triangulate a point in E-dimensional space).
4. {X(t₁), X(t₂), . . . X(t_{E+1})} are interpolated to produce an approximation \hat{X} (t*) for the discarded observation.
5. Repeating this for every t* produces a complete time series \hat{X} (t), which is an approximation based on assuming X→Y holds and using Takens' embedding idea. The Pearson correlation between the real time series X(t) and the approximant \hat{X} (t), ρ_{L}*(X, \hat{X}), is measured.
6. The process is repeated for all L* [symbol] L. This gives a relationship between how good the Takens' approximant is, (p_{L}*), versus how much data was used, (L*). The relevant measures from the test are p_{L}, the strength of the test with the most data included, and also the Spearman correlation between L* and ρ_{L}*, because in a true coupling the slope between the amount of data we have and the amount of predictive power we have should be monotonic.

For each of the 5, 138×5, 137=26, 393, 906 (self-maps are redundant) total possible cross-maps, we calculated the cross-mapping skill as a function of library length L (which cannot exceed 96, the total number of measurements) and embedding dimension E. We then performed Spearman rank-correlation on cross-mapping skill and L for each cross-mapping to isolate those that showed convergent cross-mapping (by keeping only those with p [symbol] 0.05). Initially, cross-maps that showed convergence and had their strength at maximum library (i.e., best predictive power) greater than a threshold of ρ=0.8 (representing the top 2.2%, or 606,297 pairs) were set aside as worthwhile for further analysis and experiment. This threshold was arbitrarily chosen, based on previous work¹³, but is more stringent than the natural choice of the top 5% of all CCM end-library strengths. Due to the computational cost of the ensuing tests, only the top 150,000 of these pairs (0.5% of all possible pairs, with CCM end-library strengths between 0.871 and 0.978) were further analyzed.

### Transfer Entropy

For each of the 150,000 gene pairs kept after CCM, we cross-examined our results with the unrelated method of transfer entropy¹⁹. Transfer entropy bins different areas of state space into discrete, labeled chunks, and then considers a time series of a continuous variable as a signal on the discrete states. The number of discrete states is determined by the length of the time series³⁵. Two time series are then considered causally related if the information, or entropy in one discrete signal is reduced given information about the other discrete signal. The difference in entropy between the signal by itself and the signal conditioned on the second variable is the transfer entropy. If the transfer entropy is significantly higher between two variables than between bootstrapped surrogates of the variables³⁶, then we consider them to be causally related. In this work, we examined three different methods of discretizing the state space, based on methods previously useful in biological time series³⁷. The threshold for confirmation of a CCM edge was that at least one transfer entropy binning scheme also find the edge to be a positive result. Of the original 150,000 pairs, 91,222 (60.8%) were validated by transfer entropy. The network formed by these edges, with adjacency matrix Δᵢⱼ, was used to measure graph-theoretic properties of genes for the “master score.”

### CellPaint Machine Learning Classification

To analyze the CellPaint assay readouts, we first curated the data by removing all features whose coefficient of variation across the combined set of 25yo (n=20,500 cells) and 55yo (n=6,868) cells was less than 20%, as well as any feature which was correlated with at least one other feature with Pearson ρ>0.9 (because of the extreme threshold used here, it is hardly relevant which highly correlated feature is discarded), to avoid overfitting due to multicollinearity. We then surveyed a number of supervised machine learning techniques and hyperparameter ranges. Techniques with strictly defined decision surfaces led to poor performance (linear SVM: 51.6% accuracy; Gaussian Naive-Bayes: 53.5% accuracy); while it is difficult to test all possible structures of small neural nets, we were unable to achieve accuracy significantly above 50% with multilayer perceptron-type nets. On the other hand, decision-tree-type algorithms performed well. In the end, we settled on using an adaptively-boosted classifier (AdaBoost from the scikit-learn package)—while the accuracy between random-forest and AdaBoost classifiers were nearly identical, the regularizing nature of AdaBoost helps trim the number of discriminating features to a smaller set, which we believe aids interpretability. While we investigated the effects of changing the number of estimators (equivalently, rounds of boosting), the out-of-the-box 50 estimators formed a natural elbow in a plot of accuracy vs estimator number. Our youth scores in FIG. 3 arise from train-test cross-validation, with bagging due to the class imbalance between 25yo and 55yo cells: by this we mean five AdaBoost classifiers were trained, where each classifier is trained on 70% of a dataset which is created by down sampling the set of 25yo cells such that there are equal numbers of 25yo and 55yo cells. The combination of down sampling and holding-back induces a stochastic element which ensures each classifier learns something slightly different about the dataset. The remaining 30% of each generated dataset is held back for testing, and the accuracy of the classifier is taken to be the accuracy on this test set. The youth score is given as the mean misclassification rate of the five classifiers (i.e., the number of misclassified cells divided by the number of total cells for a particular gene in the library), whereas the error bars in FIG. 3 are the standard deviation in this false-negative rate. The feature set in FIG. 4-6 are the consensus features, that is, the set of features that were used by at least 3 out of the 5 classifiers.

### Example 6. Description of In Vivo Experiments

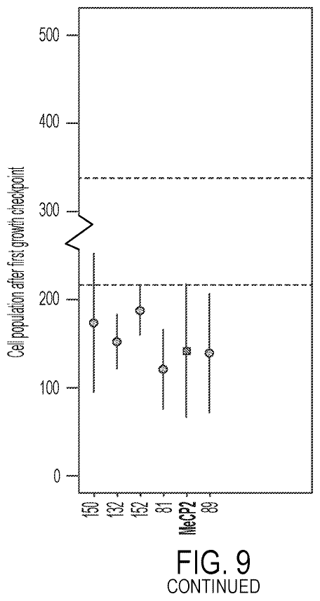

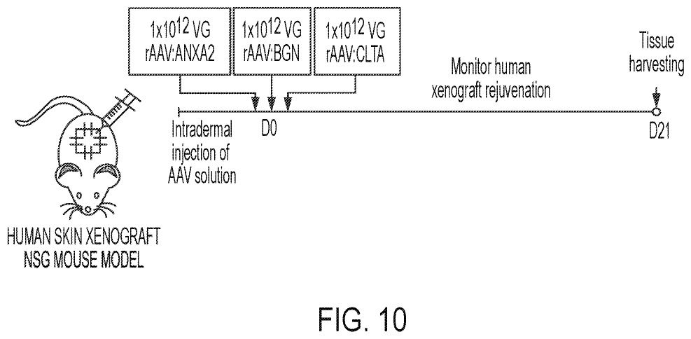

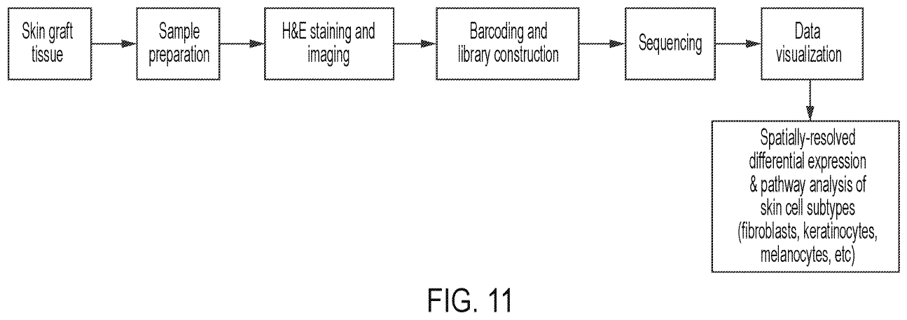

To test the effect of top candidate genes in vivo, immune-deficient NSG mice underwent surgery to graft full thickness human skin. Briefly, human skin was processed to remove subcutaneous fat, and then cut into ˜1 cm×1 cm sections. Under anesthesia, an incision was made on the lower right back of the NSG mice. The incision was spread open and then the human skin was secured in place using surgical staples. Mice recovered from this surgery for a period of 8-10 weeks, during which time the human skin graft healed and vascularized. FIG. 10 illustrates the timeline of human skin rejuvenation using AAV-based gene therapy in an NSG skin xenograft mouse model. FIG. 11 shows a flowchart illustrating the whole transcriptome analysis of human skin graft for spatially resolved mRNA sequencing across a whole fresh-frozen tissue section.

Specifically, human skin was obtained and prepared for xenograft by using adult human skin (processed with a dermatome to get ˜1 mm thickness to enable efficient graft healing). A recipient mouse (NOD.Cg-Prkdcscid Il2rgtm1Wjl/SzJ, strain #005557) was anesthetized and a ˜3 cm×3 cm area was shaved on the dorsal torso with clippers. Then, the shaved areas and adjacent fur was cleaned with 3 alternating applications of surgical scrub and 70% ethanol in a sterile field. Next, a piece of skin, approximately the same size as the grafted skin, was cut out on the back at site(s) that were shaved and cleaned, and the graft was placed in the site of skin removal. Surgical staples were applied very close together to secure the graft. Further, to ensure best grafting, forceps were used to pull the mouse and human skin with dermis sides together for the stapling. The mice were monitored as they awakened from anesthesia and doses of analgesic at 12 and 24 hours were administered after the procedure. Post-operative animals were monitored twice for the first 24 hours after surgery and then daily for a minimum of 4 days post-surgery with the Rodent Surgery Report. Animals were monitored at least once weekly for the duration of the experiment.

A cohort of 15 mice bearing human skin xenografts were prepared in this way for testing of rejuvenating genes. One of either ANXA2, BGN, or CLTA coding sequences were cloned into an rAAV vector under the control of shEF1a promoter. This recombinant vector DNA was then packaged into AAV6 serotype particles. A total of ˜1e12GC (viral genome copies) of each rAAV preparation was injected intradermally within the xenograft, spread equally over four equidistant injection locations across area of the graft, with control animals receiving an equal volume bolus of vehicle. Each treatment and control group had 3 animals. 3 weeks post-injection, mice were euthanized, and the xenografts were harvested. A portion of each xenograft was embedded and fresh-frozen in OCT to prepare for downstream sectioning ana analysis.

Raw FASTQ files emerging from the experimental pipeline were processed through SpaceRanger, software designed by 10× Genomics specifically to align FASTQs emerging from each tissue spot to a reference genome and keep the aligned reads indexed to the tissue spot. The subsequent dataset, containing a vector of RNA counts for each gene in the reference genome for each tissue spot, was analyzed using Seurat, a package implemented in the R programming language (satijalab.org/seurat). Seurat contains a suite of tools to analyze single-cell RNA sequencing data as well as in-situ/spatial RNA sequencing, with a subset of tools designed explicitly to analyze data emerging from the 10× Visium pipeline.

For each tissue sample, several quality control metrics were examined in order to disqualify tissue spots displaying severe damage or technical error from further analysis. These included common markers of such damage: the percentage of reads mapping to mitochondrial genes, the percentage of reads mapping to ribosomal RNA, the number of genes mapped, the number of reads mapped, and the number of long nuclear non-coding RNA (nuclear lncRNA), particularly MALATI. Additional quality control markers salient to work in skin, and xenograft work, included the presence of murine RNA and hemoglobin genes (HBB).

The identification of cell types with high affinity to the expression vectors seen at each tissue spot was performed using the FindTransferAnchor function within Seurat, which uses similarity between the tissue expression vector and a user-provided single-cell expression database to score the likelihood of documented cell types to have populated the focal tissue site. Here, a very large database of single-cell expression profiles was used for a robust set of skin cell types from a recent publication³⁸.

Based on an examination of the cell type scorings in conjunction with the H&E stain (hematoxylin and eosin) included in the Visium process, wherein epidermal tissue and dermal tissues can clearly be distinguished by overall color, a simple threshold was found on the cell type scorings that corresponded well to histology. This threshold mapped a tissue spot to keratinocytes (the most common cell type in the epidermis) if the affinity score for keratinocytes was above 50%, and mapped a tissue spot to fibroblasts if the affinity score for keratinocytes was less than 50%. Subsequent analyses inferring the cell cycle and differentially expressed genes were performed individually on the keratinocyte-assigned spots and the fibroblast-assigned spots, respectively.

The probability of the cells inhabiting a tissue spot being in a particular phase of the cell cycle was determined using the Seurat function CellCycleScoring, which uses the similarity between the spot's expression profile and overexpression of a predetermined set of genes for each phase included with Seurat, which are indicative of the focal phase—for instance, Synthesis phase receives a high score when an ensemble of genes related to faithful replication of DNA are highly expressed.

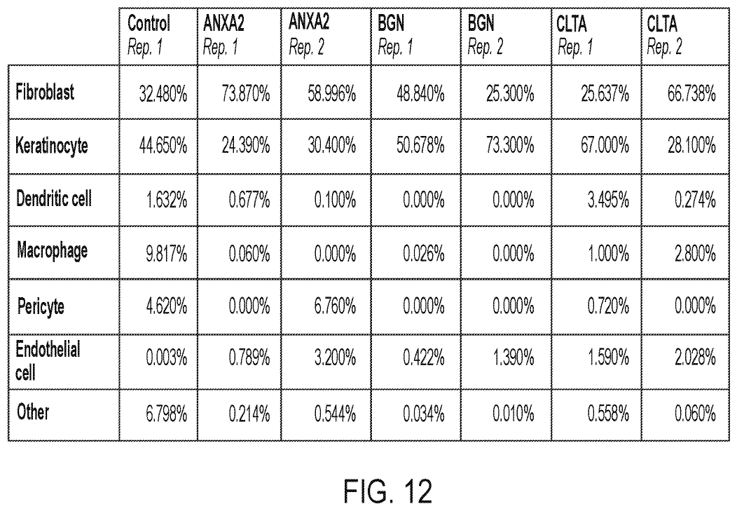

Finally, for each cell population, differential expression analysis was performed by comparing each treatment group to the control tissue using Seurat's FindMarkers function, which implements the Wilcoxon rank-sum test. Multiple-hypothesis correction was implemented via Bonferroni correction. Statistically significant differentially expressed genes in each cell population were subdivided into up- and down-regulated genes, which were then mapped to Gene Ontology (GO) biological processes using the open-source website (david.ncifcrf.gov). The GO biological processes list cutoffs were defined as Bonferroni<0.05 and a representative subset of the terms is derived using a web-based clustering algorithm Revigo (revigo.irb.hr) that relies on semantic similarity measures. FIG. 12 shows a percentage of cell types identified in control and treatment samples of Annexin A2 (ANXA2), Biglycan (BGN), and Clathrin light chain A (CLTA).

### Findings of In Vivo Experiment

To assess the effects of the genes ANXA2, BGN, and CLTA on human skin, the rAAV-vector injected human skin xenograft samples was analyzed using the 10× Genomics Visium Spatial Transcriptomics platform.

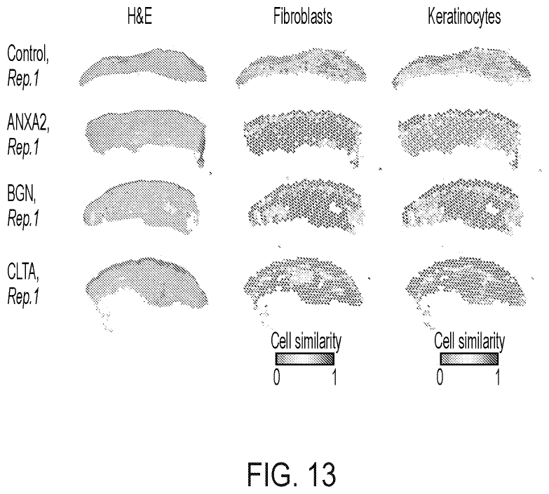

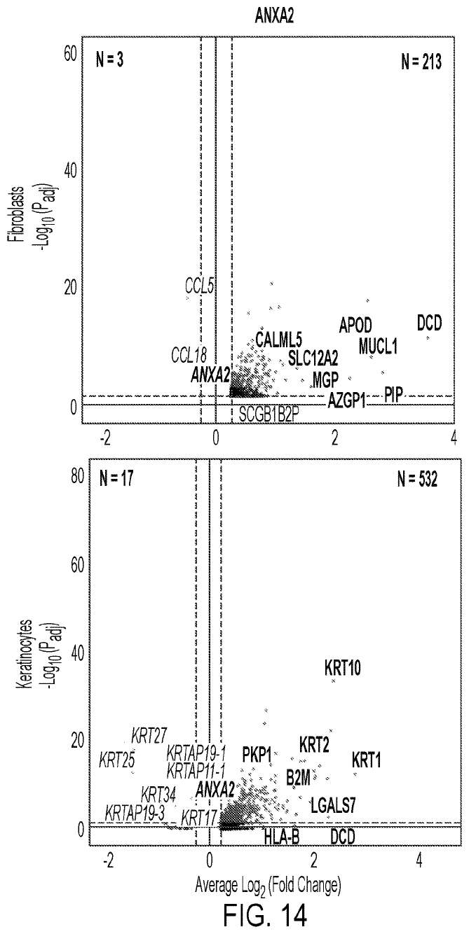

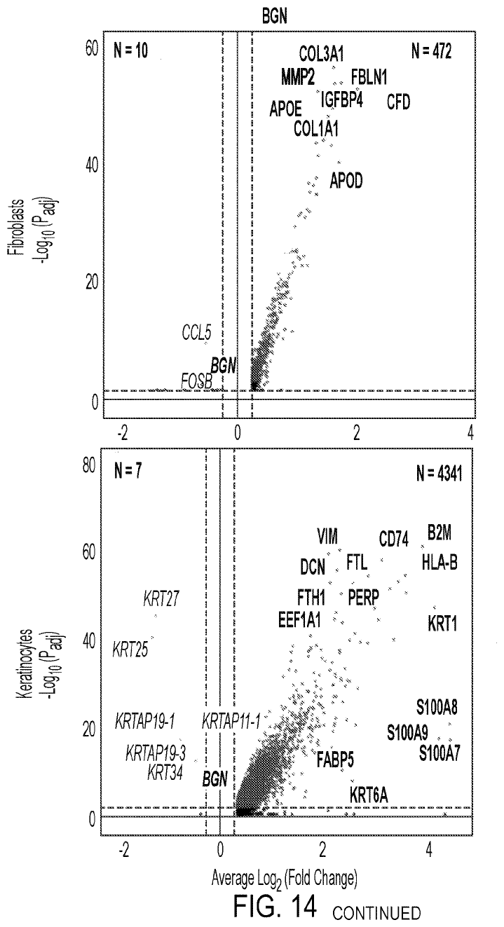

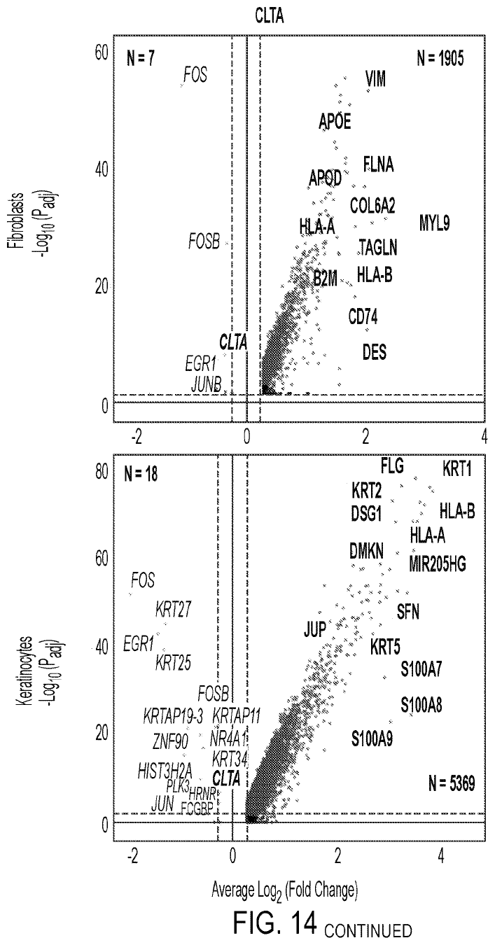

Fibroblast and keratinocyte-enriched spatial coordinates were identified by their gene expression patterns and confirmed to map to the appropriate dermal and epidermal locations (FIG. 13). Analysis of differential expression for both populations was then performed. Preliminary analysis identified a number of differentially expressed (DE) genes across all three treatments hundreds to thousands of DE genes being identified per treatment and cell type (FIG. 14). CLTA appeared to drive the largest number of DE genes in both fibroblasts and keratinocytes, and generally more DE genes were identified in keratinocytes versus fibroblasts.

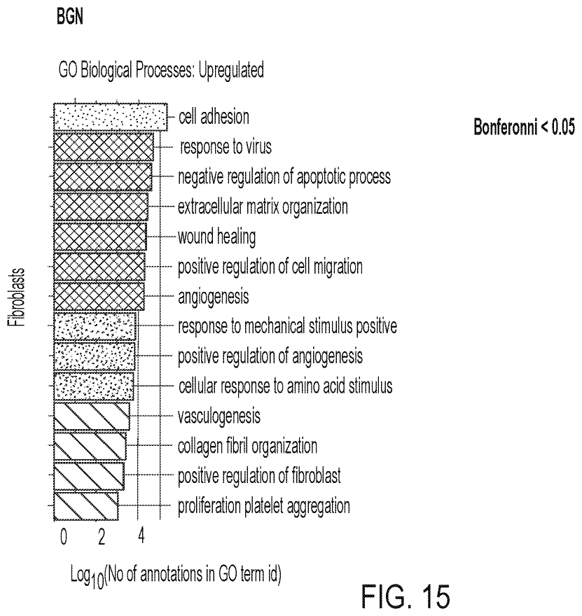

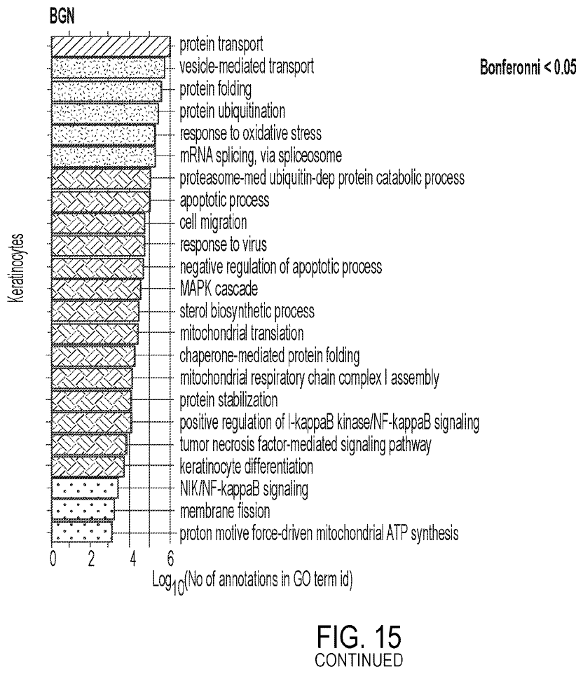

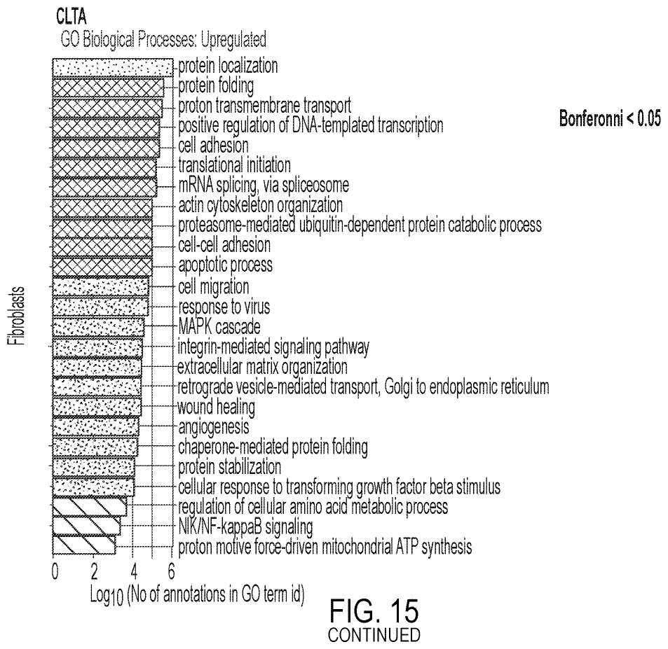

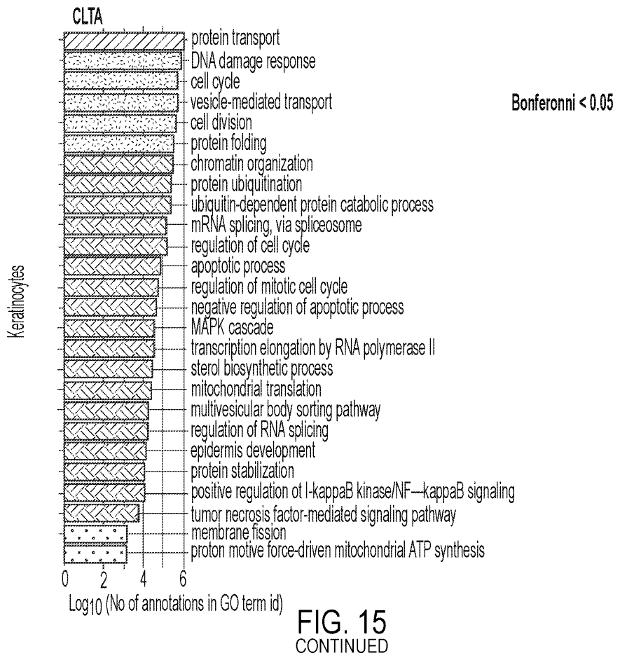

The two treatments displaying the largest number of DE genes, BGN and CLTA, were then analyzed by mapping these DE genes to annotated Gene Ontology-defined biological processes. This revealed an upregulation of cellular processes involved in wound healing, extracellular matrix organization, promotion of fibroblast survival, and fibroblast proliferation (FIG. 15, top) consistent with the restoration of cellular health, cell proliferation, and young-like cellular function identified in our initial culture-based assays.

Furthermore, despite not having been identified as restoring keratinocyte physiology, both BGN and CLTA appeared to upgregulate genes involved in epidermal stem cell maintenance, keratinocyte proliferation and migration processes compromised in aged or diseased skin. Finally, a wide range of other biological processes were upregulated, indicating a widespread effect on cellular physiology far beyond the proximal pathways and processes BGN and CLTA are embedded. This was consistent with both the in vitro finding of broad cellular phenomic rejuvenation for both genes, as well as the identification of these genes potent and broadly impactful drivers of aging due to their unique position within the inferred genetic network.

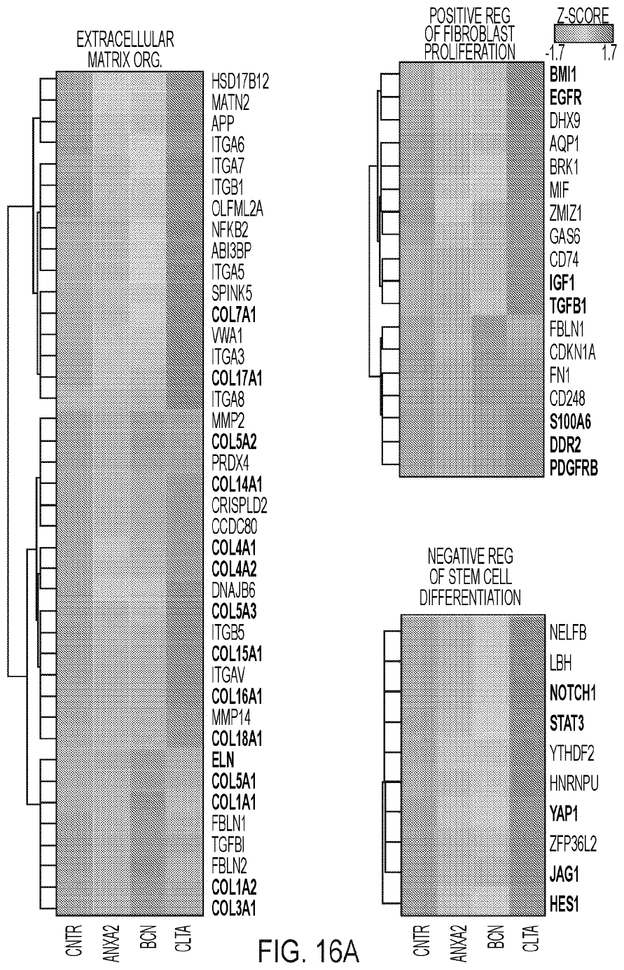

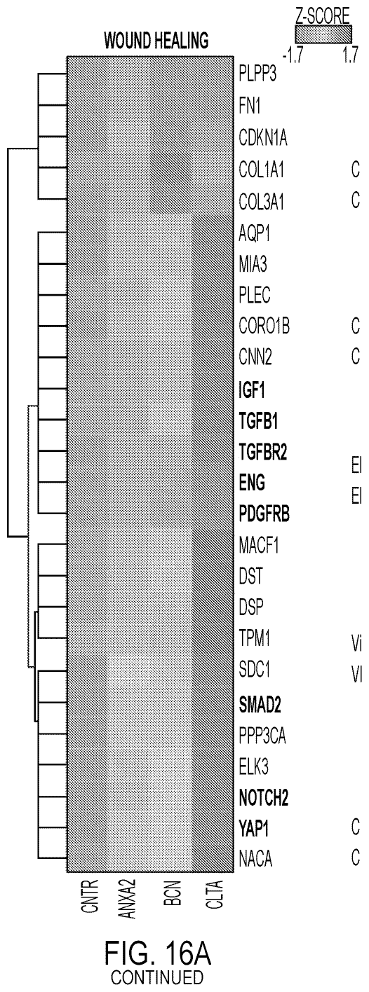

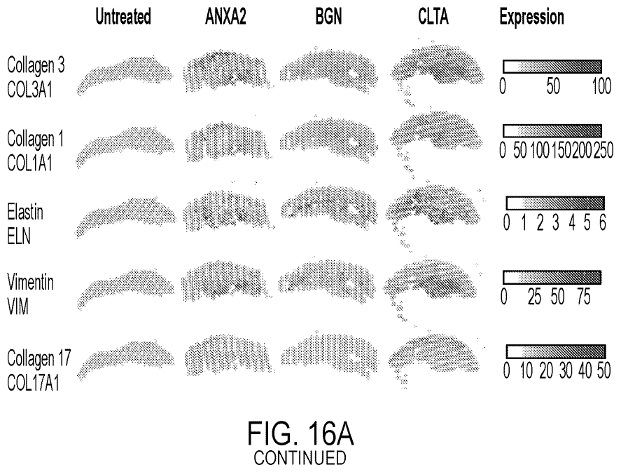

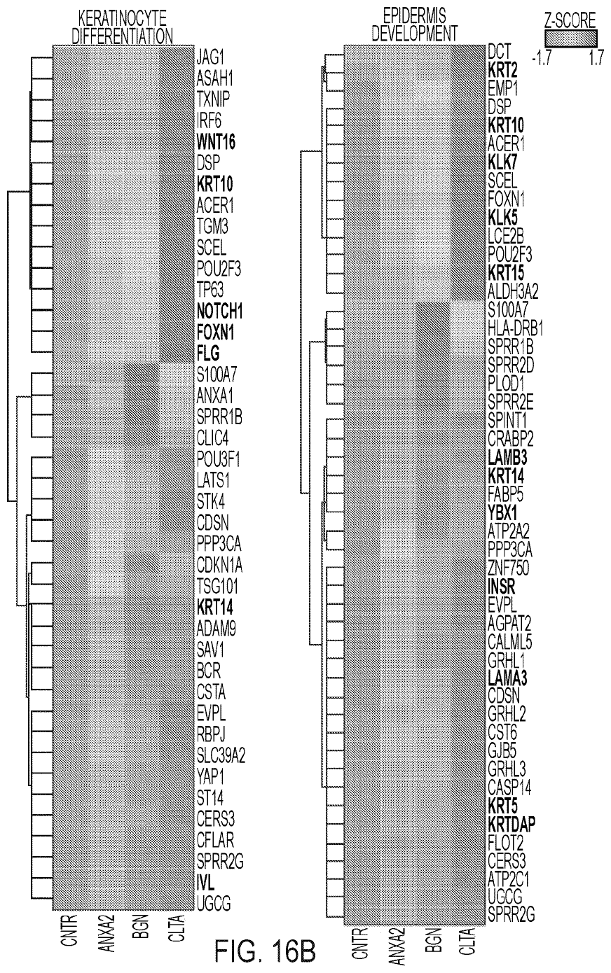

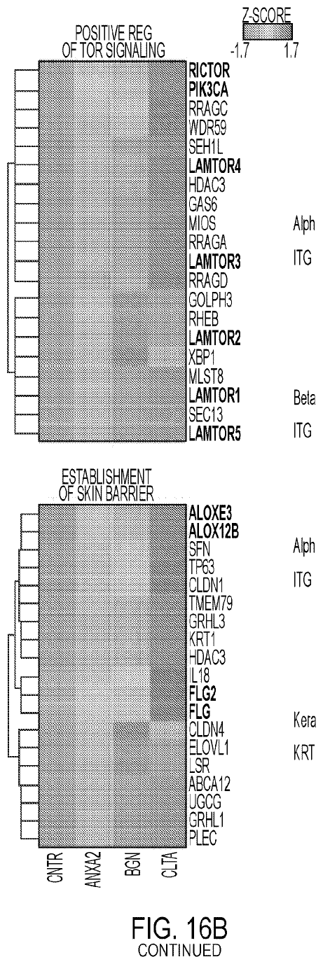

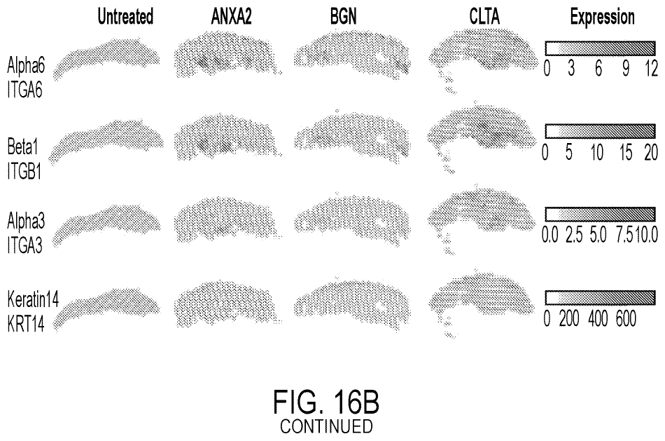

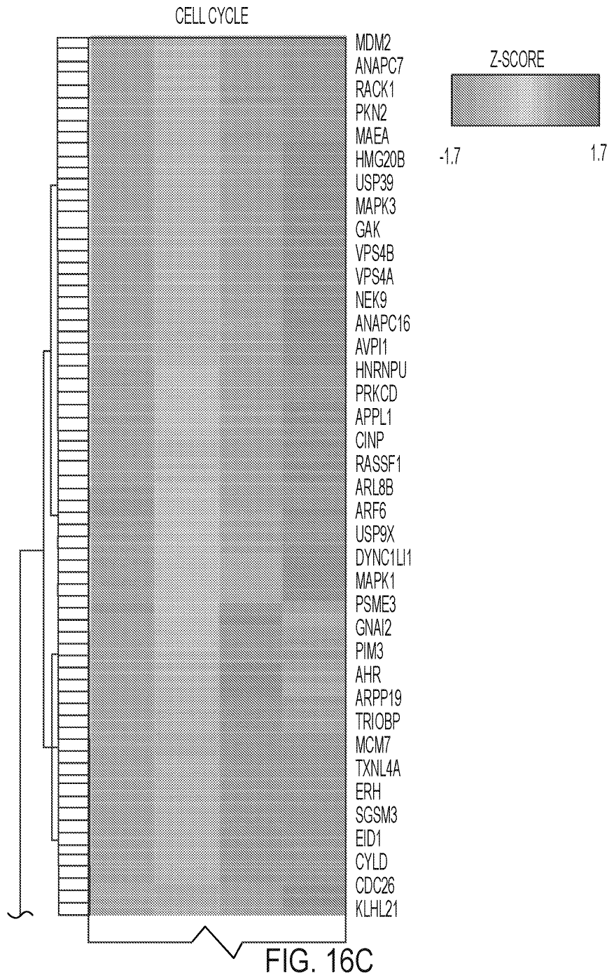

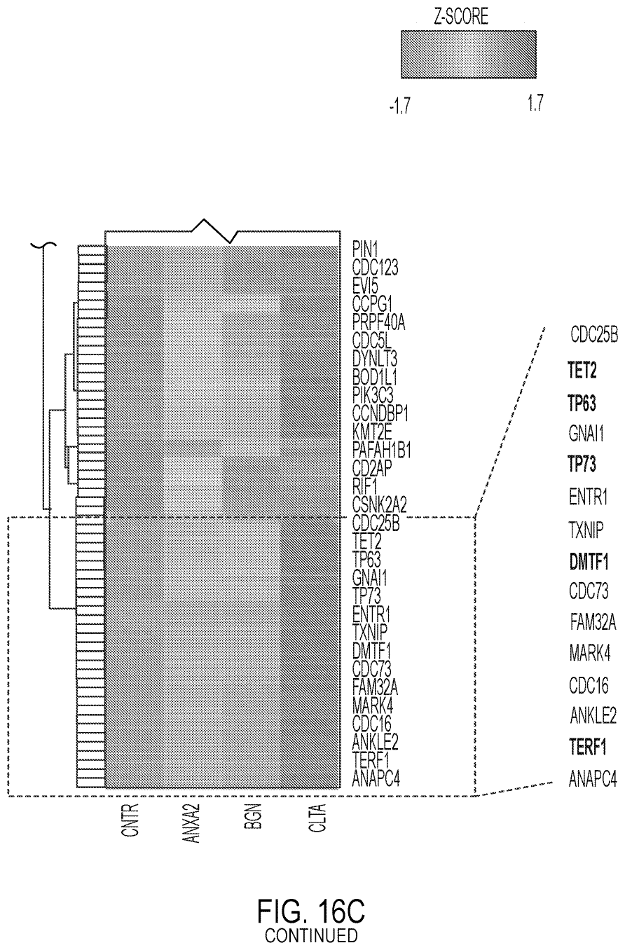

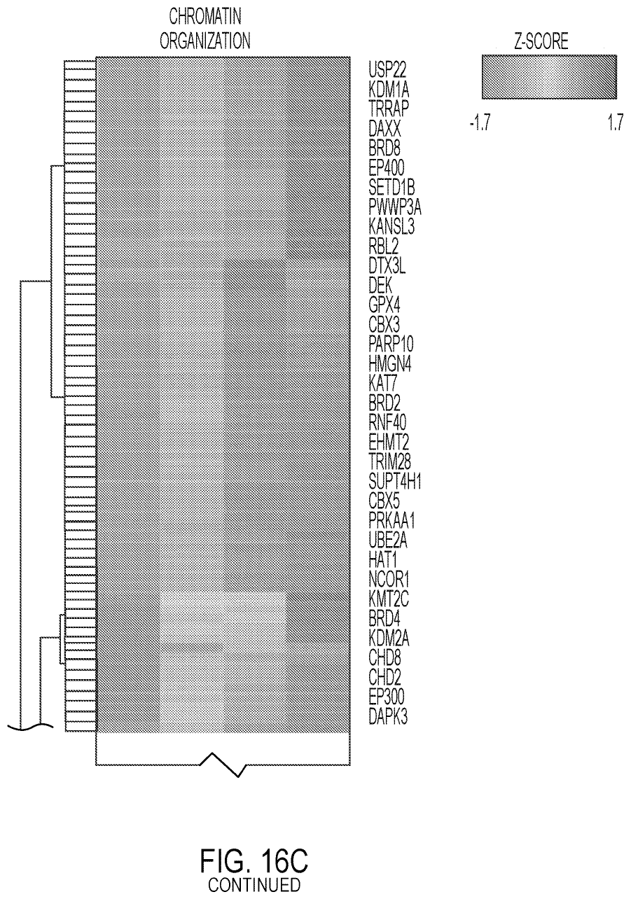

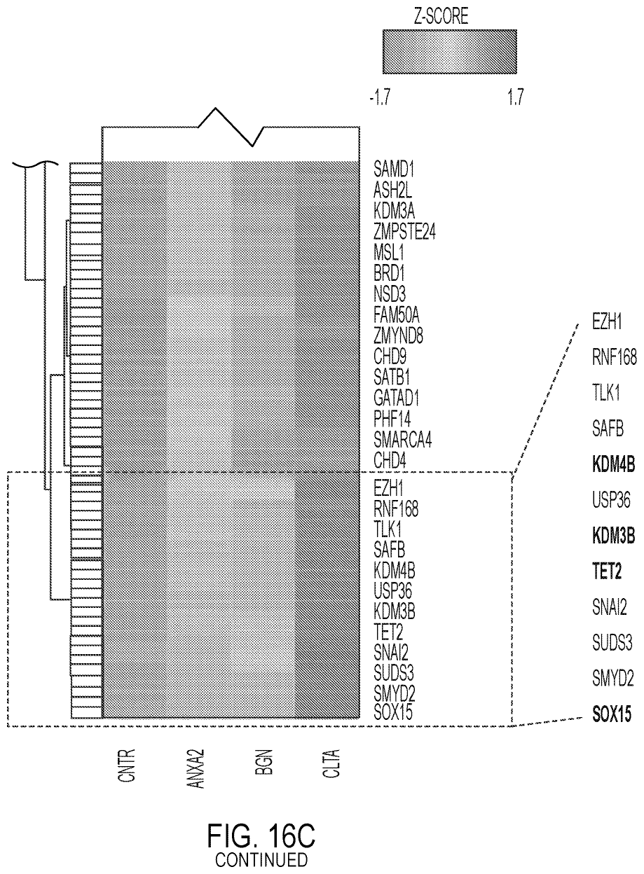

While the initial findings identified a range of per-cell rejuvenative effects, identification regarding tissue-level effects on skin health were also achieved. Specific genes (and not just broad pathways) known to mark skin cell health and alter skin cell states were assessed. As seen in FIG. 16A, more than a dozen distinct ECM components are seen to be upregulated by both BGN and CLTA treatment, alongside specific ECM adhesion molecules. Furthermore, growth and proliferation markers were identified within the fibroblast population, suggesting an enhancement of cellular proliferation and cellularization of the dermis. CLTA appeared to drive strong activation of several dermal stem cell markers involved in maintenance of stemness, contra a profile leading to stem cell niche exhaustion. And finally, CLTA, and to a less extent BGN, appeared to enhance wound healing via expression of a number of genes involved in regulation of cellular infiltration and resolution of inflammation, and process which may be ongoing even in otherwise healed xenografts. Importantly all of the above-described marker gene expression changes driven by CLTA and BGN regulate processes known to be compromised in aged skin.

FIG. 16B identifies an analogous set of changes within the keratinocytes of the epidermis. Aged and pathologic epidermis is known to decrease in cellularity, resulting from reduced keratinocyte production, disrupted differentiation, and compromised keratinization and maintenance of skin barrier function. Treatment with BGN and CLTA resulted in enhanced expression of markers of keratinocyte differentiation and proliferation, epidermal stem cell maintenance, and maturation of epidermal barrier structures. That these effects were seen in keratinocytes, despite the initial target inference and in vitro testing having been performed on fibroblasts, suggests that at least a portion of the potent network-wide effects of these genes was conserved across cell types.

Given the centrality of keratinocyte proliferation, migration and differentiation for upkeep of the essential tissue-level epidermal barrier function, it was assessed whether there were additional changes in cell cycle regulation and chromatin organization in this cell population. FIG. 16C illustrates a broad increase of cell cycle regulatory and chromatin remodeling genes. Consistent with a restoration of cell cycle progression from a depressed state, and as indicated with keratinocyte proliferation markers (FIG. 16B), both BGN and CLTA result in activation of a number of cell cycle regulatory genes, with CLTA. Both BGN and CLTA further upregulate genes involved in chromatin organization, suggesting a broad change in keratinocyte cell fate, consistent with the detection of keratinocyte maturation makers (FIG. 16B). CLTA drives the most pronounced effect in the processes, with the most potently upregulated genes (indicated by the dotted outline in FIG. 16C) including genes known to effect telomere maintenance, keratinocyte stem cell maintenance, and a host of DNA methylation and histone modifiers.

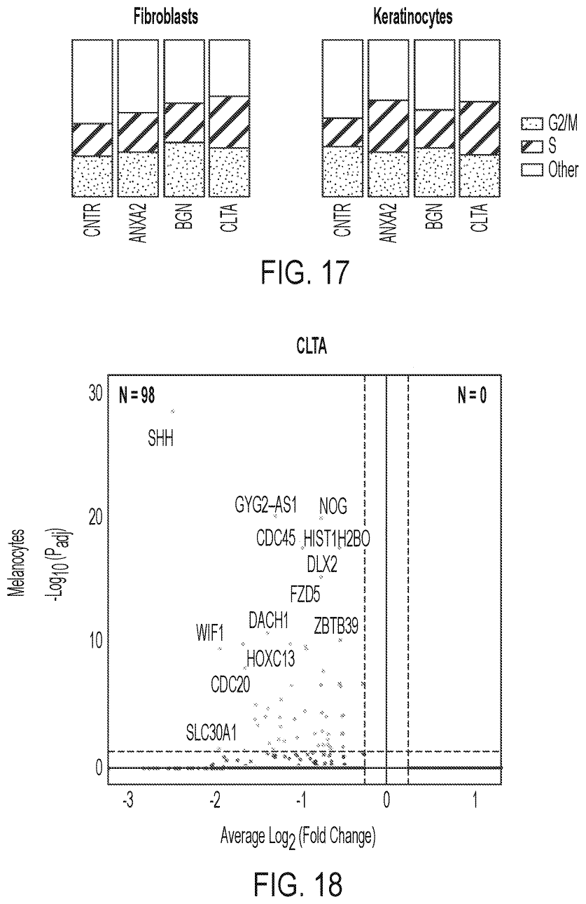

Finally, the increase in cellular proliferation across both the dermis and epidermis was assessed by inferring cell cycle states per spatial index within the Visium field. Individual spatially-index locations mapping to either keratinocyte- or fibroblast-dominated regions were assessed separately, and each spot was assigned a cell cycle phase (either G2/M, S, or other) based on the abundance of cell cycle stage-specific markers. In FIG. 17 there is an increase in the abundance of S- and G2/M-phase marker-expressing regions in fibroblasts of the dermis, with the most pronounced effects resulting from CLTA and BGN treatments, consistent with a restoration of fibroblast proliferation rates. In Keratinocytes, there is an effect on S-phase entry across treatments, with ANXA2 and CLTA resulting the largest number of S-phase keratinocyte-containing regions.

Treatment with ANXA2, CLTA, and BGN, which were identified in vitro as having a rejuvenative effect across multiple functional parameters, drove an increase in processes known to decline with age and disease, and to be generally higher in young and healthy skin. This suggests that the overall approach to predicting age-driving genes and measuring the rejuvenative effects of their perturbation is not limited to single cell functionality. Genes identified by computation and assessed for multifunctional phenomic rejuvenation in vitro appear to drive processes at an integrated tissue-level to promote skin rejuvenation and skin health enhancing biological effects.

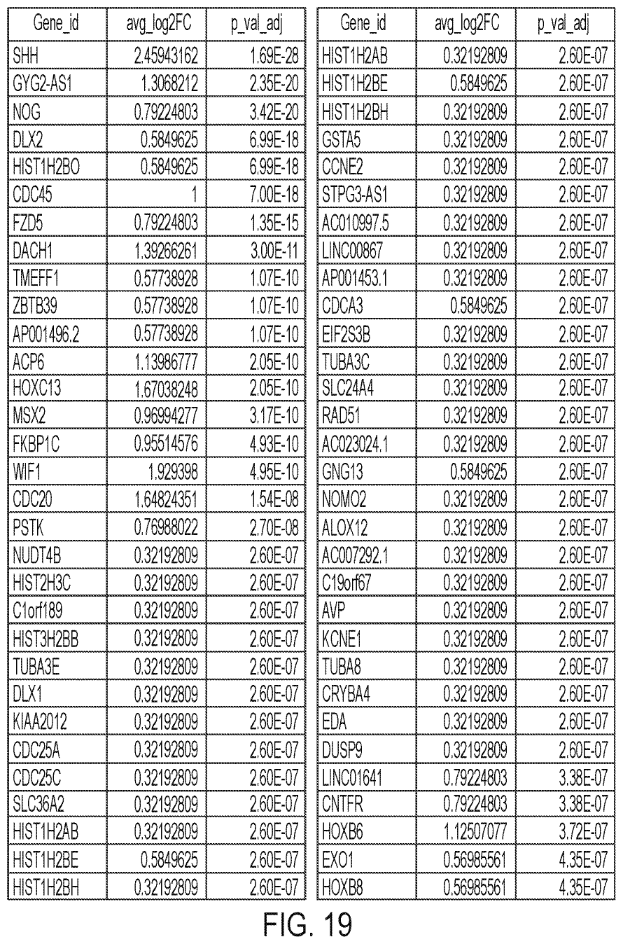

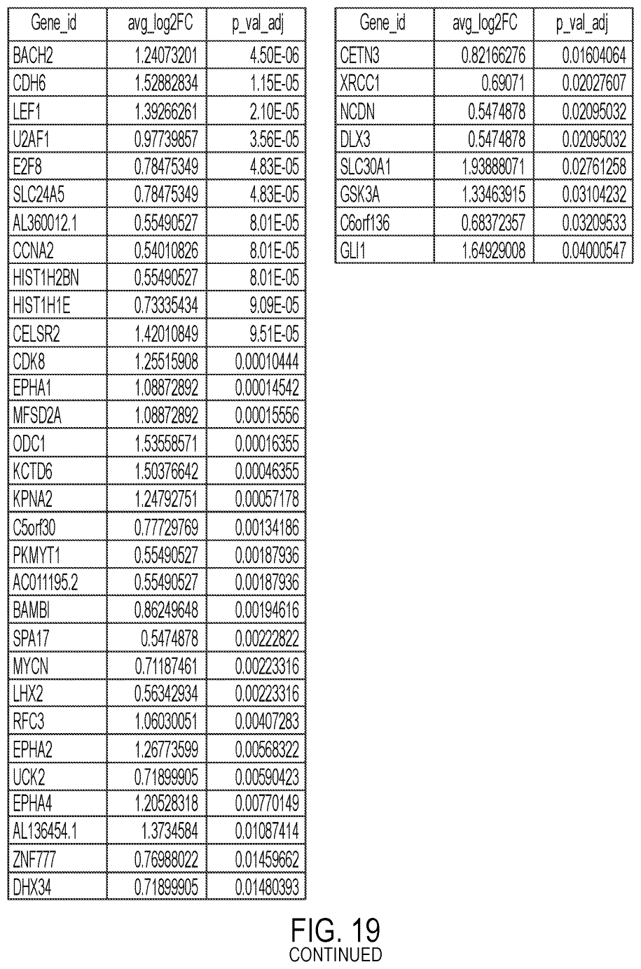

The effect of the genes on melanocyte biology was investigated separately. Melanocyte transcriptional profiles were detected in spatially indexed locations within the tissue samples and DE effects of the individual transgene treatments were assessed (FIG. 18) and all DE genes listed in FIG. 19. Among the three genes tested, CLTA showed an effect on melanocyte gene expression, driving down regulation of 98 genes. The profile of downregulated genes was consistent with healthy melanocyte function, including melanosome formation. Downregulation of several of these genes was associated with reductions in pathologic hyperpigmentation, consistent with a reduction in melanosome inflammation and pathological physiology. Changes were also detected in heterochromatin organization, suggesting a broad shift in cell state.

### REFERENCES

1. M. De Nardi, E. French, J. B. Jones, and J. McCauley, “Medical spending of the us elderly,” Fiscal studies, vol. 37, no. 3-4, pp. 717-747, 2016.
2. J. Scott, M. Ellison, and D. A. Sinclair, “The economic value of targeting aging,” Nature Aging, vol. 1, pp. 616-623, July 2021.
3. S. R. W. L. Chan and E. H. Blackburn, “Telomeres and telomerase,” Philosophical transactions of the Royal Society of London. Series B, Biological sciences, vol. 359, pp. 109-121, January 2004.
4. S. Bocklandt, W. Lin, M. E. Sehl, F. J. S\tilde{ }Aᵢnchez, J. S. Sinsheimer, S. Horvath, and E. Vilain, “Epigenetic predictor of age,” PLOS ONE, vol. 6, pp. 1-6, 06 2011.
5. C. Kenyon, J. Chang, E. Gensch, A. Rudner, and R. Tabtiang, “A C. elegans mutant that lives twice as long as wild type,” Nature, vol. 366, pp. 461-464, December 1993.
6. C. T. Murphy, S. A. McCarroll, C. I. Bargmann, A. Fraser, R. S. Kamath, J. Ahringer, H. Li, and C. Kenyon, “Genes that act downstream of daf-16 to influence the lifespan of Caenorhabditis elegans,” Nature, vol. 424, pp. 277-283, July 2003.
7. K. Podshivalova, R. A. Kerr, and C. Kenyon, “How a mutation that slows aging can also disproportionately extend end-of-life decrepitude,” Cell Reports, vol. 19, no. 3, pp. 441-450, 2017.
8. S. Ohno, “An argument for the genetic simplicity of man and other mammals,” Journal of Human Evolution, vol. 1, pp. 651-662, November 1972.
9. B. Jones, “Layers of gene regulation,” Nature Reviews Genetics, vol. 16, pp. 129-129, March 2015.
10. J. W. Tukey, “The future of data analysis,” Ann. Math. Statist., vol. 33, pp. 1-67, 1962.
11. D. Marbach, J. C. Costello, R. K\ddot{ }uffner, N. M. Vega, R. J. Prill, D. M. Camacho, K. R. Allison, A. Aderhold, R. Bonneau, Y. Chen, J. J. Collins, F. Cordero, M. Crane, F. Dondelinger, M. Drton, R. Esposito, R. Foygel, A. de la Fuente, J. Gertheiss, P. Geurts, A. Greenfield, M. Grzegorczyk, A.-C. Haury, B. Holmes, T. Hothorn, D. Husmeier, V. A. Huynh-Thu, A. Irrthum, M. Kellis, G. Karlebach, S. L'ebre, V. De Leo, A. Madar, S. Mani, F. Mordelet, H. Ostrer, Z. Ouyang, R. Pandya, T. Petri, A. Pinna, C. S. Poultney, S. Rezny, H. J. Ruskin, Y. Saeys, R. Shamir, A. S\hat{ }irbu, M. Song, N. Soranzo, A. Statnikov, G. Stolovitzky, N. Vega, P. Vera-Licona, J.-P. Vert, A. Visconti, H. Wang, L. Wehenkel, L. Windhager, Y. Zhang, R. Zimmer, and T. D. Consortium, “Wisdom of crowds for robust gene network inference,” Nature Methods, vol. 9, pp. 796-804, August 2012.
12. Y. Hirata, J. M. Amig\tilde{ }A³, Y. Matsuzaka, R. Yokota, H. Mushiake, and K. Aihara, “Detecting causality by combined use of multiple methods: Climate and brain examples,” PLOS ONE, vol. 11, pp. 1-15, 07 2016.
13. M. S. Krieger, J. M. Moreau, H. Zhang, M. Chien, J. Zehnder, and M. Craig, “A blueprint for identifying phenotypes and drug targets in complex disorders with empirical dynamics,” Cell Patterns, vol. 1, p. 100138, 2020.
14. J. G. Fleischer, R. Schulte, H. H. Tsai, S. Tyagi, A. Ibarra, M. N. Shokhirev, L. Huang, M. W. Hetzer, and S. Navlakha, “Predicting age from the transcriptome of human dermal fibroblasts,” Genome Biology, vol. 19, p. 221, December 2018.
15. G. Sugihara, R. May, H. Ye, C.-h. Hsieh, E. Deyle, M. Fogarty, and S. Munch, “Detecting causality in complex ecosystems,” Science, vol. 338, pp. 496-500, oct 2012.
16. M. Ushio, C.-h. Hsieh, R. Masuda, E. R. Deyle, H. Ye, C.-W. Chang, G. Sugihara, and M. Kondoh, “Fluctuating interaction network and time-varying stability of a natural fish community,” Nature, vol. 554, pp. 360-363, February 2018.
17. J. Runge, S. Bathiany, E. Bollt, G. Camps-Valls, D. Coumou, E. Deyle, C. Glymour, M. Kretschmer, M. D. Mahecha, J. Mu\tilde{ }noz-Marí, E. H. van Nes, J. Peters, R. Quax, M. Reichstein, M. Scheffer, B. Sch\ddot{ }olkopf, P. Spirtes, G. Sugihara, J. Sun, K. Zhang, and J. Zscheischler, “Inferring causation from time series in earth system sciences,” Nature Communications, vol. 10, p. 2553, June 2019.
18. F. Takens, “Detecting strange attractors in turbulence,” Lecture Notes in Mathematics, Berlin Springer Verlag, vol. 898, p. 366, 1981.
19. T. Schreiber, “Measuring information transfer,” Phys. Rev. Lett., vol. 85, pp. 461-464, July 2000.
20. C. J. Schmidlin, M. B. Dodson, L. Madhavan, and D. D. Zhang, “Redox regulation by nrf2 in aging and disease,” Free radical biology & medicine, vol. 134, pp. 702-707, April 2019.
21. M. C. Velarde, J. M. Flynn, N. U. Day, S. Melov, and J. Campisi, “Mitochondrial oxidative stress caused by sod2 deficiency promotes cellular senescence and aging phenotypes in the skin,” Aging (Albany NY), vol. 4, no. 1, 2012.
22. B. Salmon, G. Kim, C. Liu, J. D. Wren, C. Georgescu, A. Richardson, and R. L. Levine, “Effects of transgenic methionine sulfoxide reductase a (msra) expression on lifespan and age-dependent changes in metabolic function in mice,” Redox Biology, vol. 10, pp. 251-256, December 2016.
23. T. Lu, L. Xue, E. L. Salfati, B. H. Chen, L. Ferrucci, D. Levy, R. Joehanes, J. M. Murabito, D. P. Kiel, P.-C. Tsai, I. Yet, J. T. Bell, M. Mangino, T. Tanaka, A. F. McRae, R. E. Marioni, P. M. Visscher, N. R. Wray, I. J. Deary, M. E. Levine, A. Quach, T. Assimes, P. S. Tsao, D. Absher, J. D. Stewart, Y. Li, A. P. Reiner, L. Hou, A. A. Baccarelli, E. A. Whitsel, A. Aviv, A. Cardona, F. R. Day, N. J. Wareham, J. R. B. Perry, K. K. Ong, K. Raj, K. L. Lunetta, and S. Horvath, “Gwas of epigenetic aging rates in blood reveals a critical role for tert,” Nature Communications, vol. 9, p. 387, January 2018.
24. C. Chen, M. Zhou, Y. Ge, and X. Wang, “Sirt1 and aging related signaling pathways,” Mechanisms of Ageing and Development, vol. 187, p. 111215, April 2020.
25. L. Tasselli, W. Zheng, and K. F. Chua, “Sirt6: Novel mechanisms and links to aging and disease,” Trends in endocrinology and metabolism: TEM, vol. 28, pp. 168-185, March 2017.
26. J.-O. Pyo, S.-M. Yoo, H.-H. Ahn, J. Nah, S.-H. Hong, T.-I. Kam, S. Jung, and Y.-K. Jung, “Overexpression of atg5 in mice activates autophagy and extends lifespan,” Nature Communications, vol. 4, p. 2300, August 2013.
27. Y. Zhang, Y. Xie, E. D. Berglund, K. C. Coate, T. T. He, T. Katafuchi, G. Xiao, M. J. Potthoff, W. Wei, Y. Wan, R. T. Yu, R. M. Evans, S. A. Kliewer, and D. J. Mangelsdorf, “The starvation hormone, fibroblast growth factor-21, extends lifespan in mice,” eLife, vol. 1, p. e00065, October 2012.
28. Y. Zhao and Y.-S. Liu, “Longevity factor foxo3: A key regulator in aging-related vascular diseases,” Frontiers in Cardiovascular Medicine, vol. 8, 2021.
29. Y. Kontani, Y. Wang, K. Kimura, K.-I. Inokuma, M. Saito, T. Suzuki-Miura, Z. Wang, Y. Sato, N. Mori, and H. Yamashita, “Ucpl deficiency increases susceptibility to diet-induced obesity with age,” Aging Cell, vol. 4, pp. 147-155, June 2005.
30. C. Wang, F. Wang, Z. Li, Q. Cao, L. Huang, and S. Chen, “Mecp2-mediated epigenetic regulation in senescent endothelial progenitor cells,” Stem Cell Research & Therapy, vol. 9, p. 87, April 2018.
31. Koren, E. Reem, and A. Kimchi, “Dap1, a novel substrate of mtor, negatively regulates autophagy,” Current Biology, vol. 20, pp. 1093-1098, June 2010.
32. S. N. Chandrasekaran, H. Ceulemans, J. D. Boyd, and A. E. Carpenter, “Image-based profiling for drug discovery: due for a machine-learning upgrade?,” Nature Reviews Drug Discovery, vol. 20, pp. 145-159, February 2021.
33. G. P. Way, M. Kost-Alimova, T. Shibue, W. F. Harrington, S. Gill, F. Piccioni, T. Becker, H. Shafqat-Abbasi, W. C. Hahn, A. E. Carpenter, F. Vazquez, and S. Singh, “Predicting cell health phenotypes using image-based morphology profiling,” Molecular Biology of the Cell, vol. 32, no. 9, pp. 995-1005, 2021.
34. J. M. Phillip, P.-H. Wu, D. M. Gilkes, W. Williams, S. McGovern, J. Daya, J. Chen, I. Aifuwa, J. S. H. Lee, R. Fan, J. Walston, and D. Wirtz, “Biophysical and biomolecular determination of cellular age in humans,” Nature Biomedical Engineering, vol. 1, p. 0093, July 2017.
35. Ramos and E. Macau, “Minimum sample size for reliable causal inference using transfer entropy,” Entropy, vol. 19, p. 150, March 2017.
36. T. Schreiber and A. Schmitz, “Surrogate time series,” Physica D: Nonlinear Phenomena, vol. 142, no. 3, pp. 346-382, 2000.
37. J. Lee, S. Nemati, I. Silva, B. A. Edwards, J. P. Butler, and A. Malhotra, “Transfer entropy estimation and directional coupling change detection in biomedical time series,” BioMedical Engineering OnLine, vol. 11, no. 1, p. 19, 2012.
38. G. Reynolds, et al., “Developmental cell programs are co-opted in inflammatory skin disease,” Science, vol. 371, no. 6527, 2021.

All references, patents and patent applications disclosed herein are incorporated by reference with respect to the subject matter for which each is cited, which in some cases may encompass the entirety of the document.

TABLE 2
De-anonymization of full library of genes.

| | | | | p-value for | | | | |
|---|---|---|---|---|---|---|---|---|
| | | | | rejuvenation | | | | |
| | | | | (from 1-sided | | | | |
| | | | | Welch t-test on | | | | |
| | | | | Youth Scores for | | | | |
| | | | | perturbation | | | | |
| | | | | versus untreated | | | | |
| | | | | 55 yo cells, with | | | | |
| | | | | Bonferroni | | | | |
| | | | | correction for | | | | |
| Collection | Gene | Youth | Youth | multiple | | | MeanCell | STDCell |
| ID | Name | Score (mean) | Score(std) | hypothesis) | MeanROS | STDROS | Count | Count |
| YoungNT | YoungNT | 0.8298 | 0.0109068 | 0 | 5.21160136 | 1.428 | 338 | 168 |
| 14 | ANXA2 | 0.801689189 | 0.016510906 | 0 | 4.61869462 | 1.15816995 | 191.125 | 109.226185 |
| 110 | PRRC2B | 0.783560091 | 0.025185385 | 0 | 3.85924342 | 1.14103875 | 340.375 | 105.970441 |
| 68 | MAP4 | 0.776641221 | 0.012724071 | 0 | 3.70898275 | 1.47627876 | 288.375 | 63.2177536 |
| 135 | BGN | 0.758177215 | 0.027552121 | 0 | 3.9956732 | 1.01787686 | 375.75 | 120.891222 |
| 42 | CLTA | 0.73322053 | 0.016833769 | 0 | 3.70113841 | 1.29422408 | 328.25 | 117.846033 |
| 103 | RPS23 | 0.731704337 | 0.020232368 | 0 | 4.63224452 | 0.56965991 | 257.75 | 159.529582 |
| 154 | AKT1 | 0.691192661 | 0.016044295 | 0 | 4.20058367 | 2.29559473 | 310.25 | 43.0660829 |
| 40 | VCP | 0.657653791 | 0.016100619 | 0 | 4.14595311 | 1.61284377 | 285.5 | 70.3793294 |
| 65 | FAM129B | 0.652684145 | 0.010457124 | 0 | 4.71079197 | 1.97320912 | 183 | 43.4194657 |
| 74 | LMAN1 | 0.651131222 | 0.014949257 | 0 | 3.97296944 | 1.12144151 | 181.875 | 41.2869153 |
| 64 | GUK1 | 0.642678571 | 0.024099867 | 0 | 3.90602141 | 1.68079929 | 267.5 | 63.5590277 |
| 44 | SEC61A1 | 0.64212548 | 0.014332584 | 0 | 3.66597249 | 1.80773682 | 326.25 | 96.1271944 |
| 29 | ELL2 | 0.610027855 | 0.010859938 | 5.69E−218 | 5.70531125 | 2.44395836 | 85.75 | 17.4553001 |
| 127 | RPN1 | 0.607420706 | 0.031352986 | 0 | 4.03448931 | 1.78844267 | 332.125 | 113.367144 |
| 105 | KIF5B | 0.607303371 | 0.010381864 | 0 | 4.77033726 | 1.5871474 | 186.5 | 23.5849528 |
| 100 | MSN | 0.600373483 | 0.032911013 | 1.07E−296 | 4.32861284 | 0.89611955 | 228.875 | 45.4104545 |
| 125 | TGOLN2 | 0.597402597 | 0.039391561 | 1.16E−78 | 5.64289057 | 1.6508672 | 73.375 | 51.5629167 |
| 170 | NRF2 | 0.589928058 | 0.068553262 | 1.41E−71 | 7.45063948 | 4.91549926 | 88.625 | 54.1131627 |
| 128 | EIF4H | 0.585545518 | 0.021135838 | 0 | 5.17790633 | 0.24218422 | 255 | 111.281175 |
| 97 | RPLP1 | 0.559489051 | 0.030824836 | 0 | 3.77427256 | 1.11957798 | 251.25 | 29.3289533 |
| 99 | ASPH | 0.552380952 | 0.019087026 | 0 | 4.14657925 | 1.45877602 | 265.625 | 86.3161884 |
| 130 | SERBP1 | 0.549333333 | 0.030960907 | 4.33E−171 | 5.93335956 | 3.01261931 | 140.625 | 50.1546047 |
| 168 | SOD2 | 0.549152542 | 0.025430006 | 6.80E−122 | 7.35385444 | 4.29681153 | 93.25 | 7.10193636 |
| 22 | NACA | 0.545604964 | 0.02952317 | 0 | 4.83413462 | 2.57860595 | 248 | 26.8328157 |
| 155 | SIPA1L1 | 0.539076155 | 0.014947945 | 0 | 3.5921403 | 1.98653345 | 250.375 | 152.530274 |
| 107 | ITGA5 | 0.525192982 | 0.042722032 | 0 | 4.34703622 | 1.40319811 | 335.625 | 43.3443696 |
| 143 | BTF3 | 0.523725055 | 0.021788237 | 1.93E−197 | 5.91952353 | 3.43125722 | 133.75 | 28.1058268 |
| 3 | CAP1 | 0.513846154 | 0.021116751 | 1.20E−213 | 5.44016084 | 1.88351357 | 141.625 | 34.6732516 |
| 140 | MYDGF | 0.508549715 | 0.023234878 | 0 | 3.73086487 | 1.53258883 | 325.625 | 87.4584723 |
| 2 | YBX1 | 0.49665493 | 0.009840274 | 0 | 4.46023585 | 2.16172608 | 264 | 31.4443954 |
| 73 | MRC2 | 0.494804157 | 0.027514727 | 0 | 4.75154397 | 1.68054206 | 255.875 | 61.6024299 |
| 58 | ITGB1 | 0.472789116 | 0.018567815 | 1.09E−200 | 5.42357888 | 1.74532102 | 128.625 | 23.2966602 |
| 90 | EEF1B2 | 0.471084337 | 0.015546371 | 3.86E−176 | 5.45303163 | 2.73162967 | 102.75 | 22.8240553 |
| 15 | GAPDH | 0.464788732 | 0.039279813 | 1.15E−83 | 7.92866117 | 4.90097718 | 90.375 | 36.9964103 |
| 115 | EIF4G2 | 0.45370844 | 0.019162821 | 1.02E−273 | 5.22840495 | 2.36408135 | 174.375 | 29.4615746 |
| 27 | S100A11 | 0.449407783 | 0.010297884 | 0 | 4.53756593 | 1.52471876 | 227.875 | 77.4539178 |
| 4 | PFN1 | 0.443255814 | 0.010641917 | 0 | 4.21398397 | 1.28748864 | 181 | 42.3733407 |
| 9 | MAGED1 | 0.436860547 | 0.053981375 | 2.77E−254 | 4.14667828 | 2.1031691 | 329.625 | 34.4526396 |
| 53 | CTNNA1 | 0.435526316 | 0.015543899 | 2.23E−205 | 4.81816295 | 1.16957686 | 120.875 | 13.3551254 |
| 60 | RPL12 | 0.432706223 | 0.033620035 | 7.56E−285 | 3.70315432 | 1.77015186 | 270.375 | 80.8315185 |
| 133 | FKBP10 | 0.430027804 | 0.02404643 | 3.71E−282 | 4.86413709 | 0.87728216 | 215.5 | 57.1773557 |
| 46 | FLII | 0.426175349 | 0.036621126 | 4.72E−193 | 5.63738208 | 2.39284535 | 202.25 | 29.4862256 |
| 171 | MSRA | 0.425751073 | 0.017226294 | 0 | 4.76300228 | 1.97924811 | 250.5 | 48.551519 |
| 54 | TAGLN2 | 0.422921348 | 0.025833902 | 1.01E−155 | 6.99417544 | 4.23801289 | 135 | 26.0959767 |
| 86 | ENO1 | 0.42044293 | 0.0135603 | 9.153337e−318 | 4.66555123 | 2.69747293 | 160.625 | 41.0516062 |
| 96 | SURF4 | 0.407990012 | 0.020881433 | 1.16E−259 | 3.72432045 | 1.74507336 | 187.875 | 28.241979 |
| 147 | RSRC2 | 0.407035176 | 0.022640965 | 1.09E−177 | 7.21339135 | 4.1280303 | 143 | 54.5 |
| 33 | CLIC1 | 0.404301075 | 0.02456178 | 3.08E−85 | 7.90527975 | 4.30617483 | 78.375 | 14.3956374 |
| 144 | DAG1 | 0.402695418 | 0.022909466 | 1.00E−124 | 5.5147513 | 1.55587942 | 106.25 | 15.1471945 |
| 166 | TERT | 0.391219512 | 0.007251741 | 0 | 5.06516651 | 1.85156391 | 192 | 13.9104277 |
| 12 | LGALS1 | 0.389261745 | 0.014549989 | 5.40E−189 | 7.49770385 | 3.72498868 | 114.125 | 40.082532 |
| 172 | GSR | 0.38345021 | 0.016466379 | 0 | 5.13907643 | 1.77196241 | 199.75 | 34.9025429 |
| 39 | RPL29 | 0.376251788 | 0.009138037 | 0 | 5.61524162 | 2.71584644 | 169.75 | 37.3756271 |
| 156 | PRDX1 | 0.369395018 | 0.02128102 | 1.12E−112 | 6.63843509 | 3.58144367 | 98.25 | 30.4292212 |
| 37 | UBB | 0.36710775 | 0.024264437 | 0 | 4.60540741 | 2.38236901 | 277.75 | 30.0863341 |
| 77 | CAPN2 | 0.362264151 | 0.025262263 | 3.72E−118 | 4.13521088 | 0.79266364 | 115.75 | 24.1233808 |
| 146 | MVP | 0.356682028 | 0.010345596 | 0 | 6.29744813 | 3.41872288 | 159.875 | 52.676934 |
| 134 | Septin 10 | 0.354376658 | 0.014489655 | 2.69E−202 | 6.35766386 | 3.72035813 | 127.25 | 35.9782921 |
| 145 | Septin 8 | 0.35371179 | 0.015623182 | 0 | 5.22849199 | 3.23608211 | 215.5 | 106.320271 |
| 66 | TLN1 | 0.349292709 | 0.023790219 | 1.09E−221 | 5.47109757 | 2.22701652 | 200.25 | 45.6994256 |
| 67 | RPL23A | 0.343525741 | 0.01774361 | 7.36E−268 | 3.86170838 | 2.30942757 | 193.125 | 128.935485 |
| 17 | GNAS | 0.34137931 | 0.017936191 | 4.98E−121 | 6.68698314 | 2.71629039 | 98.875 | 15.0535503 |
| 92 | IPO5 | 0.34071511 | 0.017562045 | 0 | 4.55812337 | 2.07060654 | 233.375 | 33.6858186 |
| 149 | RPS8 | 0.340677966 | 0.019125775 | 2.84E−153 | 7.63785736 | 4.93516129 | 126.75 | 42.9323596 |
| 88 | PABPC1 | 0.337540805 | 0.016072079 | 0 | 4.64632087 | 2.2385284 | 251.875 | 41.4530985 |
| 80 | ARF1 | 0.336653386 | 0.025972918 | 3.23E−167 | 4.93191621 | 2.00965729 | 171.125 | 47.6981066 |
| 32 | RPS2 | 0.335072464 | 0.020178207 | 2.21E−263 | 4.80256031 | 1.27194424 | 214 | 49.5580468 |
| 174 | DAP | 0.328187919 | 0.004451845 | 6.38249938e−315 | 7.28522622 | 4.19242743 | 82.5 | 10.9201648 |
| 16 | RPL10A | 0.326021505 | 0.022538619 | 3.65E−101 | 6.36081592 | 2.94602979 | 102 | 22.1697993 |
| 50 | ATP5B | 0.320765027 | 0.0389706 | 6.61E−69 | 6.93239601 | 3.2821 | 104.375 | 15.499496 |
| 28 | U2AF2 | 0.318817204 | 0.021268106 | 1.98E−286 | 4.76666289 | 2.83427289 | 251.875 | 47.3588363 |
| 75 | ACTN4 | 0.318007663 | 0.031032118 | 1.71E−88 | 6.89585068 | 3.95940276 | 114.25 | 49.9768697 |
| 5 | RPS24 | 0.314641745 | 0.015005102 | 7.83E−143 | 7.84785935 | 3.87794333 | 106.375 | 29.334014 |
| 120 | ARPC5 | 0.313915858 | 0.014900795 | 4.94E−144 | 7.68688989 | 4.94039329 | 106.75 | 34.3392996 |
| 26 | FTL | 0.312121212 | 0.015075567 | 1.09E−202 | 5.32718249 | 3.62598902 | 144.25 | 58.4951921 |
| 49 | MAGED2 | 0.31041666 | 0.016298 | 6.19E−137 | 5.60283752 | 2.43196561 | 110.375 | 15.7554554 |
| 72 | RPL37 | 0.304545455 | 0.027690404 | 4.66E−65 | 9.42309511 | 5.21008813 | 83.375 | 51.070876 |
| 43 | RPSA | 0.303196347 | 0.026127342 | 1.72E−79 | 6.86692084 | 3.4038456 | 97 | 15.1492574 |
| 13 | VDAC1 | 0.3014862 | 0.019366037 | 2.53E−124 | 6.99324826 | 4.06254478 | 118.25 | 13.0264155 |
| 25 | ANXA6 | 0.3 | 0.013934223 | 1.6559337046553e−310 | 5.26060399 | 3.19729076 | 209.625 | 47.0901728 |
| 106 | RPS3A | 0.297185741 | 0.026733978 | 1.02E−127 | 5.06229295 | 2.02546443 | 155.5 | 22.2036033 |
| 138 | HP1BP3 | 0.293248945 | 0.019757021 | 7.78E−166 | 4.74727858 | 1.59593326 | 158.625 | 83.1127811 |
| 161 | LAMB3 | 0.291537545 | 0.006597546 | 0 | 4.50558644 | 1.89819773 | 237.5 | 41.2128621 |
| 70 | THRAP3 | 0.289958506 | 0.01522635 | 0 | 4.4546226 | 2.98184353 | 325.125 | 40.7413718 |
| 111 | RPL32 | 0.287749288 | 0.018286959 | 6.45E−102 | 7.9850712 | 4.57168038 | 99.875 | 15.4307931 |
| 83 | TMSB4X | 0.286896552 | 0.028883299 | 8.80E−68 | 8.23423472 | 5.33187785 | 95.75 | 26.8688947 |
| 129 | HSPA8 | 0.285897436 | 0.029403449 | 6.17E−57 | 5.81162217 | 2.56635086 | 82.75 | 17.0568901 |
| 19 | GNAI2 | 0.279596977 | 0.01427125 | 1.47E−244 | 5.28757947 | 1.89747908 | 182.25 | 26.4185068 |
| 93 | SPATS2L | 0.277390326 | 0.0098475 | 0 | 5.12057618 | 2.71652056 | 247.625 | 27.8654692 |
| 98 | PDIA4 | 0.276953125 | 0.01717862 | 1.17E−149 | 4.95484661 | 1.59702745 | 138.25 | 14.9143387 |
| 45 | RPS7 | 0.272422062 | 0.016093968 | 3.68E−148 | 7.4686479 | 4.57134224 | 132.5 | 29.0602822 |
| 101 | MYL12B | 0.267623604 | 0.018251636 | 4.49E−127 | 5.18547647 | 1.83604254 | 131 | 33.7342556 |
| 136 | CCT3 | 0.267191601 | 0.019739208 | 3.40E−114 | 6.88930256 | 3.36152309 | 127.125 | 34.2689273 |
| 20 | TPI1 | 0.265639445 | 0.011263893 | 1.20E−253 | 6.45016882 | 3.07739821 | 166.5 | 18.7082869 |
| 41 | S100A4 | 0.264576803 | 0.029712842 | 2.39E−57 | 7.2850024 | 3.47965468 | 94 | 14.6714008 |
| 52 | S100A6 | 0.255813953 | 0.033161627 | 1.85E−30 | 6.85629286 | 3.31057334 | 59.75 | 7.24137418 |
| 118 | SND1 | 0.24229249 | 0.010200386 | 6.26E−217 | 5.99250346 | 2.65117095 | 150.25 | 30.0281118 |
| 157 | DDX1 | 0.236450839 | 0.008525846 | 1.85E−209 | 7.02189249 | 3.74146106 | 129.625 | 28.89177 |
| 55 | RPS20 | 0.236170213 | 0.020759678 | 1.81E−156 | 6.56625653 | 3.99540744 | 216 | 62.4579859 |
| 48 | RPL35A | 0.231466667 | 0.014449298 | 3.11E−112 | 6.97218751 | 3.72017192 | 119.625 | 31.6501876 |
| SIRT1 | SIRT1 | 0.230725054 | 0.032764058 | 2.98E−152 | 3.920435 | 0.97654164 | 350 | 72 |
| 35 | SH3BGRL3 | 0.227436823 | 0.020100232 | 1.55E−92 | 6.32159765 | 2.41885855 | 140.125 | 14.4865239 |
| 141 | RPL23 | 0.226754386 | 0.02444364 | 8.05E−73 | 7.22861302 | 3.41710294 | 135.375 | 26.428855 |
| 173 | ATG5 | 0.224872232 | 0.018908943 | 3.31E−118 | 6.41898533 | 3.48363218 | 170.125 | 31.2187344 |
| 1 | TPM3 | 0.217428571 | 0.013474087 | 6.72E−125 | 5.4849835 | 1.71974733 | 140.75 | 85.0510876 |
| 165 | FGF21 | 0.20623608 | 0.019548426 | 1.08E−86 | 6.69388439 | 3.2679137 | 169.75 | 58.4182121 |
| 153 | TMED10 | 0.202 | 0.016679995 | 3.42E−96 | 5.45645129 | 2.57620362 | 171.75 | 39.518192 |
| 38 | EEF1A1 | 0.197938144 | 0.014340647 | 5.98E−53 | 6.78335602 | 3.57593416 | 90.625 | 10.2461883 |
| 142 | GNB1 | 0.19768595 | 0.00938517 | 9.61E−174 | 3.23233705 | 0.59921088 | 270.375 | 40.4936338 |
| 47 | EIF4G1 | 0.185359477 | 0.006887289 | 7.02E−120 | 5.26957559 | 2.60136043 | 168.875 | 41.6756449 |
| 167 | FOXO3 | 0.183890578 | 0.018135482 | 5.02E−57 | 5.99480141 | 2.94702521 | 194.125 | 41.2323826 |
| 57 | EIF1 | 0.181663113 | 0.019042314 | 1.65E−34 | 6.52763207 | 3.07531049 | 137 | 27.3313007 |
| 59 | RPLPO | 0.17993311 | 0.013277213 | 6.65E−36 | 7.29323355 | 3.64770164 | 97.25 | 22.7142576 |
| 87 | EIF5A | 0.172294372 | 0.026601371 | 4.43E−08 | 6.59455507 | 3.05857427 | 101.625 | 45.5821717 |
| 11 | XRCC6 | 0.17147651 | 0.017189595 | 1.01E−24 | 5.77938946 | 2.82104747 | 170.125 | 31.9352372 |
| 76 | RPS3 | 0.17132216 | 0.016148591 | 4.81E−27 | 6.19365374 | 4.3511224 | 173.625 | 40.5337437 |
| 6 | VIM | 0.168450704 | 0.010487048 | 4.29E−31 | 6.17056005 | 2.63212277 | 152.125 | 30.3497838 |
| 94 | COPB1 | 0.165745856 | 0.0154959 | 3.27E−13 | 6.51973169 | 2.50996304 | 150.5 | 17.5142799 |
| 117 | RPS19 | 0.162441315 | 0.012368019 | 4.56E−10 | 6.38154994 | 4.0180928 | 150.75 | 47.4730187 |
| 121 | HSPA9 | 0.16137931 | 0.019217758 | 5.07E−05 | 5.32823176 | 2.90010253 | 203.75 | 60.6954488 |
| OldNT | OldNT | 0.1534 | 0.01065082 | 156 | 5.11269323 | 2.89385334 | 217 | 46 |
| 175 | SIRT6 | 0.15 | 0.03 | | 8.1847177 | 4.333949 | 32 | 26.3980519 |
| 162 | COL3A | 0.146315789 | 0.010961754 | | 6.03629626 | 3.98235426 | 148.75 | 16.7462682 |
| 169 | UCP1 | 0.116309013 | 0.010101805 | | 5.16651408 | 2.53745318 | 129.125 | 28.4184689 |
| 176 | MeCP2 | 0.039855072 | 0.007600064 | | 7.1987151 | 5.83849376 | 142.125 | 75.3416178 |

“YoungNT” and “OldNT” represent untreated 25-year-old and 55-year-old cells, respectively.
NRF2, SOD2, MSRA, TERT, DAP, LAMB3, SIRT1, ATG5, FGF21, FOXO3, SIRT6, COL3A, UCP1, and McCP2 are control genes from the literature.

What is claimed is:

1. A method, comprising:
contacting a cell with an effective amount of a protein or a nucleic acid encoding the protein, wherein the protein is:
ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof;
wherein the effective amount is sufficient to induce cellular rejuvenation and/or regeneration of the cell.

2. A method, comprising:
administering to a subject an effective amount of a protein or a nucleic acid encoding the protein, wherein the protein is:
ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof;
wherein the effective amount is sufficient to induce cellular rejuvenation of a cell in the subject.

3. The method of claim 1 or claim 2, wherein the protein is selected from the group consisting of: ANXA2, BGN, CLTA, PRRC2B, MAP4, RPS23, AKT1, VCP, FAM129B, LMAN1, GUK1, SEC61A1, ELL2, RPN1, KIF5B, MSN, and TGOLN2.

4. The method of claim 3, wherein the protein is selected from the group consisting of: ANXA2, BGN, CLTA, PRRC2B, MAP4, and RPS23.

5. The method of any one of the preceding claims, wherein the effective amount is sufficient to reduce reactive oxygen species (ROS) abundance in the cell, compared to an untreated cell and/or wherein the effective amount is sufficient to increase the growth rate of the cell, compared to an untreated cell.

6. The method of any one of the preceding claims, wherein two or more proteins are selected, wherein the two or more proteins are encoded by two or more nucleic acids and the two or more nucleic acids are separated by a polycistronic element, optionally wherein the polycistronic element is an IRES or a 2A sequence.

7. The method of any one of the preceding claims, wherein the effective amount is sufficient to:
induce an average cellular rejuvenation of at least 5 years, at least 10 years, at least 15 years, at least 20 years, at least 25 years, at least 30 years, at least 35 years, at least 40 years, at least 45 years, or at least 50 years; and/or
increase the signal intensity of cytoplasmic actin stained with phalloidin and measured by microscopy, the signal intensity of the Golgi apparatus/apparati stained with wheat germ agglutinin and measured by microscopy, the signal intensity of the plasma membrane stained with wheat germ agglutinin and measured by microscopy, the signal intensity of extranuclear DNA stained with Hoechst 33342 and measured by microscopy, the signal intensity of mitochondria stained with MitoTracker Deep Red and measured by microscopy, the signal intensity of the endoplasmic reticulum stained with concanavalin A and measured by microscopy, or the peripheral cellular signal intensity of RNA stained with SYTO 14 and measured by microscopy; and/or
decrease cytoplasmic volume as measured by microscopy, decrease cell volume as measured by microscopy, decrease cell surface area as measured by microscopy, or decrease the signal intensity of nuclear DNA stained with Hoechst 33342 and measured by microscopy; and/or
induce an average cellular rejuvenation of at least 20 years.

8. The method of any one of the preceding claims, wherein the cell is an adult stem cell, optionally wherein the adult stem cell is hematopoietic stem cell, epithelial stem cell, neuronal stem cell or mesenchymal stem cell, optionally, wherein mesenchymal stem cell is fibroblast, myocyte, adipocyte, chondrocyte, or osteocyte, optionally wherein the hematopoietic stem cell is a T cell or NK cell, and optionally wherein the T cell is a CD4⁺CD8⁺ cell, CD4⁺ cell (Th1, Th2, Th17, or Treg), a naive T cell, a central memory T cell, or an effector memory T cell.

9. The method of any one of the preceding claims, wherein the cell is an ectoderm, endoderm, mesoderm or germ cell, optionally wherein the ectoderm cell is keratinocyte, pigment cell or neuronal cell, optionally, wherein the endoderm cell is a liver cell, lung cell, pancreatic cell or thyroid cell, optionally wherein the mesoderm cell is a cardiac muscle cell, skeletal muscle cell, smooth muscle cell, kidney tubule cell or a red blood cell, and optionally, wherein the germ cell is an egg or sperm cell.

10. The method of any one of the preceding claims, wherein the cell is selected from the group consisting of: fibroblasts, hematopoietic stem cells, endothelial cells, chondrocytes, skeletal muscle stem cells, keratinocytes, mesenchymal stem cells. and corneal epithelial cells, optionally wherein the cells are fibroblasts, and optionally wherein the fibroblasts are human dermal fibroblasts.

11. The method of any one of the preceding claims, wherein the protein is a human, canine, feline, bovine, ovine, caprine, equine, murine, porcine or pachyderm protein.

12. The method of claim 2, wherein the protein is delivered to skin tissue layers and structures including stratum corneum, epidermis, basement membrane, dermis, hair follicles, blood vessels, and sebaceous glands or and eccrine glands.

13. The method of any one of the preceding claims, wherein the nucleic acid comprises a heterologous promoter operably linked to an open reading frame, optionally wherein the heterologous promoter is a constitutive promoter or an inducible promoter, optionally wherein the regulatory sequence comprises a cell-specific promoter or a tissue-specific promoter, optionally wherein the regulatory sequence comprises a promoter selected from the group consisting of: an hEfla promoter, an shEfla promoter (or truncated hEfla promoter), a CAG promoter (such as cytomegalovirus, chicken beta-actin intron, splice acceptor of the rabbit beta-globin gene), a CMV promoter, an hAAT promoter, a thyroid hormone-binding globulin promoter, an albumin promoter, a thyroxin-binding globulin (TBG) promoter, a hepatic control region (HCR)-ApoCII hybrid promoter, a CASI promoter, an HCR-hAAT hybrid promoter, an hAAT promoter combined with mouse albumin gene enhancer (Ealb) element, and an apolipoprotein E promoter, optionally wherein the nucleic acid is operably linked to a 3′ untranslated region for RNA stability and expression in mammalian cells, and optionally wherein the 3′ untranslated region comprises a WPRE sequence, a WPRE3 sequence, an SV40 late polyadenylation signal (e.g., truncated), an HBG polyadenylation signal, a rabbit beta-globin polyadenylation signal, a bovine bgpA, an ETC polyadenylation signal, or any combination thereof.

14. The method of any one of the preceding claims, wherein the protein is delivered in a viral vector, optionally wherein the viral vector is an AAV vector, and optionally wherein the AAV vector is derived from an AAV serotype selected from the group consisting of: AAV1, AAV2, AAV3, AAV4, AAV5, AAV6, AAV6.2, AAV7, AAV8, AAV9, AAV10, AAV11, AAV12, AAV13, AAVrh8, AAVrh10, and AAVrh32.

15. The method of any one of the preceding claims, further comprising contacting the cell with the protein or administering the protein directly to the subject or further comprising contacting the cell with the protein or administering to the subject the nucleic acid comprising an open reading frame encoding the protein.

16. The method of any one of the preceding claims, wherein the nucleic acid is delivered in a non-viral vector or a viral vector.

17. The method of any one of the preceding claims, wherein the contacting comprises transfecting the cell.

18. The method of any one of the preceding claims, wherein the protein comprises a sequence that is at least 90% identical to any one of SEQ ID NOs: 1-6.

19. The method of any one of the preceding claims, wherein the nucleic acid comprises an amino acid sequence that is at least 90% identical to any one of SEQ ID NOs: 7-12.

20. A method, comprising:
overexpressing in a cell a nucleic acid encoding a protein, wherein the protein is:
ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof;
wherein the overexpressing produces an effective amount of the protein in the cell, and wherein the effective amount is sufficient to induce cellular rejuvenation in the cell.

21. A cell, comprising:
an engineered nucleic acid encoding a protein, wherein the protein is:
ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IP05, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, HSPA9, or any combination thereof.

22. A pharmaceutical composition comprising the cell of claim 21 and a pharmaceutically-acceptable excipient and/or polymeric carrier.

23. A pharmaceutical composition comprising:
(a) a recombinant vector genome comprising one or more transgenes encoding one or more polypeptide sequences selected from ANXA2, BGN, CLTA, PRRC2B, MAP4, RPS23, AKT1, VCP, FAM129B, LMAN1, GUK1, SEC61A1, ELL2, RPN1, KIF5B, MSN, TGOLN2, EIF4H, RPLP1, ASPH, SERBP1, NACA, SIPA1L1, ITGA5, BTF3, CAP1, MYDGF, YBX1, MRC2, ITGB1, EEF1B2, GAPDH, EIF4G2, S100A11, PFN1, MAGED1, CTNNA1, RPL12, FKBP10, FLII, TAGLN2, ENO1, SURF4, RSRC2, CLIC1, DAG1, LGALS1, GSR, RPL29, PRDX1, UBB, CAPN2, MVP, SEPT10, SEPT8, TLN1, RPL23A, GNAS, IPO5, RPS8, PABPC1, ARF1, RPS2, RPL10A, ATP5B, U2AF2, ACTN4, RPS24, ARPC5, FTL, MAGED2, RPL37, RPSA, VDAC1, ANXA6, RPS3A, HP1BP3, THRAP3, RPL32, TMSB4X, HSPA8, GNAI2, SPATS2L, PDIA4, RPS7, MYL12B, CCT3, TPI1, S100A4, S100A6, SND1, DDX1, RPS20, RPL35A, SH3BGRL3, RPL23, TPM3, TMED10, EEF1A1, GNB1, EIF4G1, EIF1, RPLP0, EIF5A, XRCC6, RPS3, VIM, COPB1, RPS19, and HSPA9,
wherein the vector genome is configured to express the one or more transgene at a level sufficient to induce tissue rejuvenation and/or regeneration; and
(b) a pharmaceutically-acceptable excipient and/or polymeric carrier.

24. A method, comprising administering the pharmaceutical composition of claim 23 to skin of a subject via intraepidermal, transepidermal, intradermal, transdermal, subcutaneous, intramuscular, or topical administration.

25. A method for measuring the age of a cell, comprising:
(a) contacting a cell with a first reagent capable of recognizing cytoplasmic actin, with a second reagent capable of recognizing Golgi apparatus/apparati, with a third reagent capable of recognizing plasma membrane, with a fourth reagent capable of recognizing extranuclear DNA, with a fifth reagent capable of recognizing mitochondria, with a fifth reagent capable of recognizing endoplasmic reticulum, with a sixth reagent capable of recognizing RNA.
(b) determining the morphological and/or functional fingerprint of the cell based on the intensity of a signal associated with the first, second, third, fourth, fifth, and sixth binding reagent and the intensity of a signal associated with the at least second binding reagent;
(c) and identifying the age of the cell based upon a machine learning algorithm of extracted features.

26. A method for defining a rejuvenation index, called a ‘Youth score’, wherein the cell is contacted with a perturbant which induces a cell's transition from aged (reference) cell state to younger (altered) cell state, comprising:
(a) a change in a cell state, function and/or expression between the totality of unperturbed cells and the totality of perturbed cells; and
(b) comparing the cell's transition fingerprint thereby quantifying the cellular transition due to the perturbation.


SEQUENCE LISTING (12 sequences)

SEQ ID NO: 1 - protein, 339 aa, Homo sapiens [sequence omitted]

SEQ ID NO: 2 - protein, 2229 aa, Homo sapiens
MSDRLGQITKGKDGKSKYSTLSLFDKYKGKSVDAIRSSVIPRHGLQSLGKVAAARRMPPPANLPSLKSENKGNDPNIVIVPKDGTGWANKQDQQDPKSSSATASQPPESLPQPGLQKSVSNLQKPTQSISQENTNSVPGGPKSWAQLNGKPVGHEGGLRGSSRLLSFSPEEFPTLKAAGGQDKAGKEKGVLDLSYGPGPSLRPQNVTSWREGGGRHIISATSLSTSPTELGSRNSSTGDGAPSSACTSDSKDPSLRPAQPVRKGASQFMGNVYHPPTYHDMLPAFMCSPKSSENQGTVERGSFPLPQLRLEPRVPFRQFQMNDQDGKENRLGLSRPLRPLRQLVERAPRPTIINAENLKGLDDLDADADDGWAGLHEEVDYSEKLKFSDDEEEEEVVKDGRPKWNSWDPRRQRQLSMSSADSADAKRTREEGKDWAEAVGASRVVRKAPDPQPPPRKLHGWAPGPDYQKSSMGSMFRQQSIEDKEDKPPPRQKFIQSEMSEAVERARKRREEEERRAREERLAACAAKLKQLDQKCKQARKAGEARKQAEKEVPWSPSAEKASPQENGPAVHKGSPEFPAQETPTTFPEEAPTVSPAVAQSNSSEEEAREAGSPAQEFKYQKSLPPRFQRQQQQQQQEQLYKMQHWQPVYPPPSHPQRTFYPHHPQMLGFDPRWMMMPSYMDPRITPTRTPVDFYPSALHPSGLMKPMMPQESLNGTGCRSEDQNCVPPLQERKVTPIDSPPVWSPEGYMALQSKGYPLPHPKSSDTLAMDMRVRNESSFSASLGRAGGVSAQRDLFEERGEEYLSAFDKKAQADFDSCISSQRIGQELLFPPQENVQDAGAPGGHTQNLRCSPLEPDFVPDEKKPECGSWDVSHQPETADTAHGVERETPREGTAFNISSWDKNGSPNKQPSSEPEWTPEPRSSSSQHPEQTGRTRRSGPIKKPVLKALKVEDKEKELEKIKQELGEESTRLAKEKEQSPTAEKDEDEENDASLANSSTTTLEDKGPGHATFGREATKFEEEEKPDKAWEARPPRESSDVPPMKRNNWIFIDEEQAFGVRGQARGRGRGFREFTFRGRPAGGNGSGLCGGGVLGARSIYCSSQRSGRGRGLREFARPEDCPRAKPRRRVASETHSEGSEYEELPKRRRQRGSENGNEGSLLEREESTLKKGDCRDSWRSNKGCSEDHSGLDAKSRGPRAFGRALPPRLSNCGYGRRTFVSKESPHWQSKSPGSSWQEYGPSDTCGSRRPTDRDYVPDSYRHPDAFGGRGFEDSRAEDKRSFFQDEHVADSENAENRPFRRRRPPRQDKPPRFRRLRQERESLGLWGPEEEPHLLAGQWPGRPKLCSGDKSGTVGRRSPELSYQNSSDHANEEWETASESSDFSERRERREGPGSEPDSQVDGGLSGASLGEKKELAKRSFSSQRPVVDRQSRKLEPGGFGEKPVRPGGGDTSPRYESQQNGTPLKVKRSPDEALPGGLSGCSSGSGHSPYALERAAHASADLPEASSKKAEKEAKLAAPRAGEQGEAMKQFDLNYGSAIIENCGSSPGEESEVGSMVGEGFIEVLTKKQRRLLEEERRKKEQAVQVPVKGRGLSSRIPPRFAKKQNNLCLEQGDVTVPGSSLGTEIWESSSQALPVQAPANDSWRKAVTAFSSTETGSAEQGFKSSQGDSGVDLSAESRESSATSSQRSSPYGTLKPEEMSGPGLAEPKADSHKEQAPKPSEQKDSEQGSGQSKEHRPGPIGNERSLKNRKGSEGAERLQGAVVPPVNGVEIHVDSVLPVPPIEFGVSPKDSDFSLPPGSASGPTGSPVVKLQDALASNAGLTQSIPILRRDHHIQRAIGLSPMSFPTADLTLKMESARKAWENSPSLPEQSSPGGAGSGIQPPSSVGASSGVNYSSFGGVSMPPMPVASVAPSASMPGSHLPPLYLDGHVFASQPRLVPQTIPQQQSYQQAAAAQQIPISLHTSLQAQAQLGLRGGLPVSQSQEIFSSLQPFRSQVYMHPSLSPPSTMILSGGTALKPPYSAFPGMQPLEMVKPQSGSPYQPMSGNQALVYEGQLSQAAGLGASQMLDSQLPQLTMPLPRYGSGQQPLILPQSIQLPPGQSLSVGAPRRIPPPGSQPPVLNTSREPSQMEMKGFHFADSKQNVPSGGPVPSPQTYRPSSASPSGKPSGSAVNMGSVQGHYVQQAKQRVDEKPSLGAVKLQEAPSAASQMKRTGAIKPRAVKVEESKA

SEQ ID NO: 3 - protein, 1152 aa, Homo sapiens
MADLSLADALTEPSPDIEGEIKRDFIATLEAEAFDDVVGETVGKTDYIPLLDVDEKTGNSESKKKPCSETSQIEDTPSSKPTLLANGGHGVEGSDTTGSPTEFLEEKMAYQEYPNSQNWPEDTNFCFQPEQVVDPIQTDPFKMYHDDDLADLVFPSSATADTSIFAGQNDPLKDSYGMSPCNTAVVPQGWSVEALNSPHSESFVSPEAVAEPPQPTAVPLELAKEIEMASEERPPAQALEIMMGLKTTDMAPSKETEMALAKDMALATKTEVALAKDMESPTKLDVTLAKDMQPSMESDMALVKDMELPTEKEVALVKDVRWPTETDVSSAKNVVLPTETEVAPAKDVTLLKETERASPIKMDLAPSKDMGPPKENKKETERASPIKMDLAPSKDMGPPKENKIVPAKDLVLLSEIEVAQANDIISSTEISSAEKVALSSETEVALARDMTLPPETNVILTKDKALPLEAEVAPVKDMAQLPETEIAPAKDVAPSTVKEVGLLKDMSPLSETEMALGKDVTPPPETEVVLIKNVCLPPEMEVALTEDQVPALKTEAPLAKDGVLTLANNVTPAKDVPPLSETEATPVPIKDMEIAQTQKGISEDSHLESLQDVGQSAAPTFMISPETVTGTGKKCSLPAEEDSVLEKLGERKPCNSQPSELSSETSGIARPEEGRPVVSGTGNDITTPPNKELPPSPEKKTKPLATTQPAKTSTSKAKTQPTSLPKQPAPTTIGGLNKKPMSLASGLVPAAPPKRPAVASARPSILPSKDVKPKPIADAKAPEKRASPSKPASAPASRSGSKSTQTVAKTTTAAAVASTGPSSRSPSTLLPKKPTAIKTEGKPAEVKKMTAKSVPADLSRPKSTSTSSMKKTTTLSGTAPAAGVVPSRVKATPMPSRPSTTPFIDKKPTSAKPSSTTPRLSRLATNTSAPDLKNVRSKVGSTENIKHQPGGGRAKVEKKTEAAATTRKPESNAVTKTAGPIASAQKQPAGKVQIVSKKVSYSHIQSKCGSKDNIKHVPGGGNVQIQNKKVDISKVSSKCGSKANIKHKPGGGDVKIESQKLNFKEKAQAKVGSLDNVGHLPAGGAVKTEGGGSEAPLCPGPPAGEEPAISEAAPEAGAPTSASGLNGHPTLSGGGDQREAQTLDSQIQETSI

SEQ ID NO: 4 - protein, 368 aa, Homo sapiens
MWPLWRLVSLLALSQALPFEQRGFWDFTLDDGPFMMNDEEASGADTSGVLDPDSVTPTYSAMCPFGCHCHLRVVQCSDLGLKSVPKEISPDTTLLDLQNNDISELRKDDFKGLQHLYALVLVNNKISKIHEKAFSPLRKLQKLYISKNHLVEIPPNLPSSLVELRIHDNRIRKVPKGVFSGLRNMNCIEMGGNPLENSGFEPGAFDGLKLNYLRISEAKLTGIPKDLPETLNELHLDHNKIQAIELEDLLRYSKLYRLGLGHNQIRMIENGSLSFLPTLRELHLDNNKLARVPSGLPDLKLLQVVYLHSNNITKVGVNDFCPMGFGVKRAYYNGISLFNNPVPYWEVQPATFRCVTDRLAIQFGNYKK

SEQ ID NO: 5 - protein, 248 aa, Homo sapiens
MAELDPFGAPAGAPGGPALGNGVAGAGEEDPAAAFLAQQESEIAGIENDEAFAILDGGAPGPQPHGEPPGGPDAVDGVMNGEYYQESNGPTDSYAAISQVDRLQSEPESIRKWREEQMERLEALDANSRKQEAEWKEKAIKELEEWYARQDEQLQKTKANNRVADEAFYKQPFADVIGYVTNINHPCYSLEQAAEEAFVNDIDESSPGTEWERVARLCDFNPKSSKQAKDVSRMRSVLISLKQAPLVH

SEQ ID NO: 6 - protein, 143 aa, Homo sapiens [sequence omitted]

SEQ ID NO: 7 - DNA, 1017 bp, Homo sapiens
  source 1..1017: mol_type = genomic DNA
atgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacgttgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgac

SEQ ID NO: 8 - DNA, 6687 bp, Homo sapiens
  source 1..6687: mol_type = genomic DNA
atgtccgatcgtttggggcaaattaccaagggcaaggatgggaaaagcaagtactcgactctcagcctgtttgataagtataaaggaaaatcagtagacgcgattagatcctcagttattcctagacatggcttacagagtcttgggaaagttgctgcagcccggcgcatgccaccgcctgcaaacctgccaagcttgaagtctgaaaacaaaggaaacgaccccaacatcgtgatagtacccaaggacgggacgggatgggcaaacaagcaggatcagcaagacccaaagagttccagtgcgacggcctctcagccgccggagtcgctgccgcagccgggtttgcagaaatctgtctccaatttgcagaaaccgacacagtcaatcagtcaggagaatacaaattcagtgccaggtggaccaaagtcatgggcacagctgaatggaaagccagtaggacacgaaggtggtttaaggggctcaagccgactgttatccttctctcccgaggaatttccgacgctgaaagcagctggagggcaggacaaggctggcaaagaaaagggcgtcttagatctgtcgtatgggccaggaccaagcctccgccctcagaatgtgacaagctggagggagggcggtgggcgacacataatttctgccacgtctctgagcacctccccaactgagctgggcagcaggaactcgagtacgggagatggagccccctcctcggcatgtaccagcgattctaaggacccctctctccgcccggctcagcctgtccgaaaaggggcttcacagttcatgggaaatgtataccacccacctacataccatgacatgcttcctgcttttatgtgttcgccgaagtcatcagaaaaccagggtacagtggaacgaggctcttttccccttcctcagctccgccttgaacctcgagttccttttagacagttccagatgaatgaccaagacggaaaagaaaacaggctgggattgtctcgcccactccgcccactaaggcagctggtggagcgggcaccacggcccaccattatcaatgcggaaaacctgaagggccttgacgatctggacgccgatgccgatgatggctgggcaggcctccatgaagaagtggactattctgagaaactgaagttcagtgatgatgaagaggaggaagaagttgtgaaggacggcaggccaaagtggaacagttgggaccctaggaggcagcggcagttgtcaatgagctctgcagacagtgcggacgctaagcggactcgagaggaagggaaggactgggctgaagcagtgggtgcgtcccgtgtggtccgaaaggcgccagaccctcagccaccgcccaggaagcttcatggctgggcaccaggccctgactaccagaagtcatcaatgggcagcatgttccggcaacagtccatcgaggacaaggaggacaagcccccaccaaggcagaagttcattcagtcagagatgtccgaggcggtggagcgagcccgaaagcgccgggaagaagaggagcgccgagcccgggaggagaggctggccgcctgtgctgccaaactcaagcagctggaccagaagtgtaagcaggcacgaaaggcaggtgaggcccggaagcaggcagagaaggaagtgccctggtctccaagtgctgagaaggcatctccccaggaaaacggccctgctgtccacaaaggctccccagaattccctgcccaagagacccccaccacattcccagaagaggcacccacagtgtccccagcagtggcacagagcaacagcagtgaggaagaggccagagaggctgggtcccctgcacaggagttcaagtatcagaagtcccttcctccccgattccagcgccagcagcagcaacaacagcaggagcagctgtacaagatgcagcactggcagccggtgtaccccccgccgtcccacccccagcgcaccttttacccacaccacccccagatgttgggcttcgatcccaggtggatgatgatgccttcctacatggacccacgtatcacgcccactcggaccccggtggacttctacccctccgccctgcatccctcaggactgatgaagcccatgatgccccaggagtccctcaatgggacaggctgtcgctctgaggatcagaactgtgtgcccccactccaagaaagaaaagtgacccccatcgactcaccccctgtgtggagcccagagggctacatggcactgcagagcaagggctacccgctcccgcacccgaagtcgagtgacaccttggctatggacatgcgtgtcaggaatgaaagctctttctctgcctcactcggaagggcagggggcgtaagtgctcagcgcgatctctttgaggagagaggggaggagtacttgagtgcttttgacaagaaggcccaagcagactttgacagctgtatctcttctcaaagaataggccaggagcttttgtttccaccccaagaaaatgttcaggatgcaggtgctcctgggggtcacacccaaaacctcaggtgttccccattggagcctgactttgtcccagatgagaaaaagccagagtgtggcagttgggatgttagccaccagccagagaccgctgacacagcccatggtgttgagcgggagacaccccgggaggggacggcctttaacatctcctcctgggacaagaacgggagccccaacaaacagccatcctcggagcctgaatggactcccgagccccggagctccagcagccagcacccggagcagacgggcaggacccggaggtcgggacccatcaagaaaccagtcctgaaagccctcaaggtggaagacaaggagaaggagcttgagaagattaagcaggagctaggggaggagagtacccggctggccaaggagaaggagcagagccccacggcagaaaaggatgaggacgaagagaacgatgcctctctggccaactcctccaccaccactttggaggacaaaggccctggccatgccacttttggccgcgaggccaccaaatttgaagaggaggagaaacctgacaaggcctgggaagccagacccccacgagagtccagcgatgttccccccatgaagagaaataactggatctttattgatgaggagcaagcctttggggtcagaggacaggcccggggccggggccgtggtttcagagagttcacttttcgtggtcggcctgctggcggaaatgggagcggcctctgtggtgggggggtcctgggggcccgcagcatctactgcagcagtcagcgcagcggccgtggccggggcctgcgagagtttgcgcggccagaggactgccccagagccaagccccgacggagagttgccagtgagacccatagcgagggctcagagtatgaagaacttcccaagcgccgccggcagaggggctccgagaacgggaatgaaggctcgctcctggagagggaggagagcaccttgaagaagggcgactgcagagattcttggcggtccaacaaggggtgctctgaggaccacagcggtctagatgccaagagccgaggccctcgggcctttgggcgagccctccctccccggctgagcaattgcgggtatggacggagaaccttcgtctccaaagagtcaccccactggcagagcaaaagtccaggcagctcttggcaggaatatggcccttccgacacatgcggatcccggcgacctacagacagagactatgtcccagattcctacagacaccctgacgcatttggtggccggggctttgaggacagccgcgcggaggacaagagatccttcttccaagatgaacacgtggcagattctgaaaatgcagagaaccggcccttcaggagaaggcgccccccacgccaagataagccccctcgattccggcgcctccggcaagagcgggagtccctgggcctgtggggacccgaggaggagccccacctgctggcaggtcagtggccaggcaggcccaaactgtgttctggggacaagagtggcactgtgggccgcaggtcccctgagctctcctaccagaactcctccgatcacgccaatgaggagtgggagacggcctccgaaagcagcgacttcagcgagcggcgggagcggcgggaaggccctgggtccgagcccgactcccaggtggatggtggcctgtcgggggctagtttgggtgagaagaaggagctggccaagaggagcttctccagtcagagacccgtggttgacagacagagccgaaagctggagccgggagggtttggggagaagcccgttaggccaggtggtggtgacacctcccctcgctatgagagccaacagaatgggacgcctttgaaagtgaaaagatccccagacgaggccttgcctggaggtcttagtggctgcagcagtgggagtggccactccccctatgccctggagcgggcagcccatgccagtgctgaccttcccgaagcctccagtaaaaaggcagagaaggaggccaagttggctgctccgagggcaggtgaacagggagaggccatgaaacagtttgacctgaactatggaagtgccatcattgaaaattgcgggtccagccccggggaggagagtgaggtgggttctatggtgggcgaaggcttcatcgaagtcctgaccaagaagcagcgccgcctgctggaggaagagagaagaaagaaggagcaggccgtgcaggtgcctgtcaaaggtcgaggcctttcctcccgtattcctcctcgatttgcaaaaaagcagaacaacttatgtctggagcaaggtgacgtgaccgtgcctggcagcagcctgggcactgagatctgggagagcagcagccaggctctccctgtgcaggccccagccaacgactcctggaggaaagctgtcactgccttcagcagcaccgagactggctctgcggagcagggttttaagagcagccagggagatagtggcgttgacttgagtgccgagtctcgggagtcgtctgcgacctcctcgcagcgcagctccccatatgggactctgaagccagaggagatgagcgggcccggcctggcggaacccaaggccgacagccacaaggagcaggctccaaagccatctgagcagaaggattcagaacaaggctctggacagagcaaggagcacagaccaggacccatcggcaacgagcgttctctgaaaaacagaaagggctcggagggggccgagcggctgcaaggggctgtcgtcccgcctgttaacggggtggagattcacgtggactccgtgctgcctgtgccacccattgaatttggagtcagtccaaaagactccgatttcagcttgccacctggttctgcctctggtcctactgggagtccagttgttaaacttcaggatgccttggccagtaatgcagggttaacacagagtatccccatcctgcggcgggaccatcacatccagagggccatcggtctctccccaatgtccttccccaccgccgaccttactctgaagatggagtctgcgcgcaaggcttgggaaaactcccccagtttgccggagcagagctctccaggcggcgctggctcaggcatccagcctccatcctctgtgggtgcctccagcggggtcaactacagctccttcggtggagtgtccatgccacccatgcctgtggcctctgtagcaccttctgcttctatgccaggcagccacctcccgcccctgtacctggatggccatgtgtttgcaagtcagccccggctggttcctcaaacgatacctcagcagcagagttaccaacaggccgccgctgcccagcagatcccgatctcccttcacacatctctgcaggcacaagctcagcttggactgaggggtgggcttcctgtgtcccagtcccaggagatcttcagctccttgcagcccttcagatctcaggtgtacatgcaccccagcctgtcaccgcccagcaccatgatcctctctgggggcacagccttgaagcctccatactcggcgttcccaggcatgcagcccttggagatggtgaagccgcagtctggctcaccctaccagcccatgagcgggaaccaagccctggtctacgagggccagctcagccaggctgctggcctgggtgcctcccagatgttggactcccagctcccacagctgaccatgccactgcctcggtacggctccgggcagcagccactgatcctgccccagtctattcagctgccacctgggcagagcctctccgttggggccccccgaaggattcctccgcccgggtcccagccgccagtcctgaacaccagcagagagccctctcagatggagatgaaaggcttccactttgccgacagtaaacagaatgtcccttcaggaggccccgtgccatcgccacagacctacaggcctagctctgctagccccagtgggaagccctctggatcagcagttaacatgggctctgtgcagggacactacgtgcaacaggcaaaacaacgagtggatgagaaacccagcctgggagccgtgaagctgcaggaggccccctcggctgcctcccagatgaagcgaaccggagcgatcaagcctcgggctgtcaaagtggaggagagtaaggcc

SEQ ID NO: 9 - DNA, 3456 bp, Homo sapiens
  source 1..3456: mol_type = genomic DNA
atggccgacctgagcctggctgacgccctgaccgagcccagccccgacattgagggcgagatcaagagagacttcatcgccacattagaggccgaggccttcgacgacgtggtgggcgagacagtgggcaagacagactacatccccctgctggacgtggacgagaagaccggcaacagcgaatccaagaagaagccctgcagcgagacaagccagatcgaggatacccccagcagcaagcctacactgctggccaatggcggccacggagtggagggcagcgataccacaggaagccccacagagttcctggaggagaagatggcctaccaggagtaccccaacagccagaactggcccgaggacaccaacttctgcttccagcccgaacaggtggtggaccccatccagaccgaccccttcaagatgtaccacgacgacgacctggccgatctggtgtttcccagctctgccaccgccgacaccagcatctttgccggccagaacgacccccttaaggacagctacggcatgtctccctgcaacaccgccgtggttcctcagggctggagcgtggaagccctgaatagccctcacagcgagtctttcgttagccccgaggccgttgctgaaccacctcagcccacagccgtgccactggagcttgccaaagaaatcgagatggccagcgaagaaaggcctcctgcccaggccctggagatcatgatgggcctgaagaccaccgacatggccccaagcaaggagactgagatggctctggccaaggacatggccctggccacaaagaccgaggtggccctggccaaggacatggagtcccccaccaagctggacgtgacactggccaaagacatgcagccaagcatggagagcgacatggccctggtgaaggatatggagctgcctaccgagaaggaggtggctctggttaaggacgtgaggtggcccaccgaaaccgacgtgagcagcgccaagaacgtggtgctgccaaccgagactgaagtggcccccgctaaggatgtgaccctgctgaaggagacagagagagccagccctatcaagatggacctggccccttccaaggacatgggcccccccaaggagaataagaaggaaaccgagcgggccagccccatcaagatggatctggccccctccaaggatatgggccctcctaaggagaacaagatcgtgcccgccaaggacctggtgctgctgagcgagatcgaggtggcccaggccaacgacatcatctctagcaccgagattagcagcgccgagaaagtggccctgagctccgagacagaagtggccctggcccgggacatgacactgcctcccgaaacaaacgtgatcctgaccaaggacaaggccctccctctggaggccgaggtggcccctgttaaggatatggcccagctgcctgagacagagatcgcaccagccaaggacgtggctccctctacagtgaaggaggtgggcctgctgaaggacatgtctcccctgagcgaaaccgagatggccctgggcaaagacgtgacccccccacccgagacagaggtggtgctgatcaagaacgtgtgcctgccaccagagatggaggttgccctgacagaggaccaggtgcctgccctgaaaaccgaagctccccttgccaaggatggcgttctgaccctcgctaataacgtgactcctgctaaggacgtgccccccctgtccgagactgaggccacccccgtgcccatcaaagatatggagatcgcccagacccagaagggcattagcgaggacagccacctggaatctctgcaggacgtgggccagagcgccgcccctaccttcatgatctctcccgagactgtgaccggaaccggcaagaaatgcagcctgcctgccgaggaagacagcgtgctcgagaagctgggcgagagaaagccttgcaactcccagcccagcgagctgtctagcgaaacaagcggaattgcccggcctgaggagggaagacccgtggtgtctggcaccggcaacgatatcaccacccctcccaacaaggagctgcccccctctcctgagaagaagaccaagcccctggctacaacccagcccgccaagacctctaccagcaaggccaaaacccaacccaccagcctgcccaagcagcctgctcccacaacaatcggcggacttaataagaagcccatgagccttgccagcggcctggtgccagccgctcctcctaaaagacctgccgtggcctctgccagaccctccattctgcccagcaaggatgtgaagcctaagcccatcgccgatgccaaggctcccgagaagagagctagccctagcaagccagccagcgctcccgccagcagaagcggaagcaagagcacacagacagtggctaagacaacaaccgctgccgcagtggcttctaccggcccctcctctagatcccccagcacactgctgcccaaaaagcccaccgccatcaagacagagggaaagcccgctgaggtgaagaagatgaccgccaagagcgtgcccgccgacctttccaggcccaagtctacctccacaagctctatgaagaagacaaccacactgtctggaaccgccccagccgccggcgtggtgcctagcagagtgaaggccacccctatgcccagcagaccttctacaacccctttcatcgacaagaagcccacaagcgccaagccctccagcaccacccccagactgagcagactggccaccaataccagcgcccctgacctgaagaatgtgcggagcaaagtgggctccaccgagaacatcaagcaccagcccggcggcggcagagccaaggtggagaagaaaacagaagccgccgccacaaccagaaaacccgagtctaacgctgtgaccaaaacagccggccccattgccagcgcccagaagcagccagccggcaaggtgcagatcgtcagcaagaaggtgtcctacagccacatccagagcaagtgcggcagcaaggacaatatcaagcacgtgccaggcggcggcaacgtgcagatccagaacaagaaggtggacatcagcaaggtgagctccaagtgcggctccaaggccaacatcaagcacaagcctggcggcggcgacgtgaagatcgagagccagaagctcaacttcaaggagaaggcccaggctaaggtgggcagccttgataacgttggccacttacctgccggcggcgctgtgaagaccgaaggcggaggatctgaggcccctctgtgtcctggacctcctgctggagaggagcctgctatttctgaagctgctcctgaggctggcgctcctacatctgcatctggcctgaacggacaccccaccctgagcggcggcggagatcagagagaagctcagaccctggactctcagatccaggagacaagcatc

SEQ ID NO: 10 - DNA, 1104 bp, Homo sapiens
  source 1..1104: mol_type = genomic DNA
atgtggcccctgtggcgcctcgtgtctctgctggccctgagccaggccctgccctttgagcagagaggcttctgggacttcaccctggacgatgggccattcatgatgaacgatgaggaagcttcgggcgctgacacctcaggcgtcctggacccggactctgtcacacccacctacagcgccatgtgtcctttcggctgccactgccacctgcgggtggttcagtgctccgacctgggtctgaagtctgtgcccaaagagatctcccctgacaccacgctgctggacctgcagaacaacgacatctccgagctccgcaaggatgacttcaagggtctccagcacctctacgccctcgtcctggtgaacaacaagatctccaagatccatgagaaggccttcagcccactgcggaagctgcagaagctctacatctccaagaaccacctggtggagatcccgcccaacctacccagctccctggtggagctccgcatccacgacaaccgcatccgcaaggtgcccaagggagtgttcagcgggctccggaacatgaactgcatcgagatgggcgggaacccactggagaacagtggctttgaacctggagccttcgatggcctgaagctcaactacctgcgcatctcagaggccaagctgactggcatccccaaagacctccctgagaccctgaatgaactccacctagaccacaacaaaatccaggccatcgaactggaggacctgcttcgctactccaagctgtacaggctgggcctaggccacaaccagatcaggatgatcgagaacgggagcctgagcttcctgcccaccctccgggagctccacttggacaacaacaagttggccagggtgccctcagggctcccagacctcaagctcctccaggtggtctatctgcactccaacaacatcaccaaagtgggtgtcaacgacttctgtcccatgggcttcggggtgaagcgggcctactacaacggcatcagcctcttcaacaaccccgtgccctactgggaggtgcagccggccactttccgctgcgtcactgaccgcctggccatccagtttggcaactacaaaaag

SEQ ID NO: 11 - DNA, 744 bp, Homo sapiens
  source 1..744: mol_type = genomic DNA
atggctgaattagatcctttcggagctcctgctggcgctcctggaggtcctgctttaggaaatggagttgctggagccggagaagaagatcctgctgcggcctttttagctcagcaagagagcgagattgctggcattgagaatgacgaggcctttgctattcttgacggaggagctcctgggcctcaacctcatggagaacctcctgggggtccggatgctgttgatggagtaatgaatggtgaatactaccaggaaagtaatggtccaacagacagttatgcagctatttcacaagtggatcgactgcagtcagagcctgaaagtatccgtaaatggagagaagaacaaatggaacgcttggaagcccttgatgccaattctcggaagcaagaagcagagtggaaagaaaaggcaataaaggagctagaagaatggtatgcaagacaggacgagcagctacagaaaacaaaagcaaacaacagggtggccgatgaagctttctacaaacaacccttcgctgacgtgattggttatgtcacaaacataaaccatccttgctacagcctagaacaggcagcagaagaagcctttgtaaatgacattgacgagtcgtccccaggcactgagtgggaacgggtggccaggctttgtgactttaaccctaagtctagcaagcaggccaaagatgtctcccgcatgcgctcagtcctcatctccctcaagcaagccccgctggtgcac

SEQ ID NO: 12 - DNA, 429 bp, Homo sapiens
  source 1..429: mol_type = genomic DNA
atgggcaagtgtcgtggacttcgtactgctaggaagctccgtagtcaccgacgagaccagaagtggcatgataaacagtataagaaagctcatttgggcacagccctaaaggccaacccttttggaggtgcttctcatgcaaaaggaatcgtgctggaaaaagtaggagttgaagccaaacagccaaattctgccattaggaagtgtgtaagggtccagctgatcaagaatggcaagaaaatcacagcctttgtacccaatgacggttgcttgaactttattgaggaaaatgatgaagttctggttgctggatttggtcgcaaaggtcatgctgttggtgatattcctggagtccgctttaaggttgtcaaagtagccaatgtttctcttttggccctatacaaaggcaagaaggaaagaccaagatca